(19) United States
(12) Patent Application Publication
(10) Pub. No.: US 2024/0189218 A1
(43) Pub. Date: Jun. 13, 2024

(54) COMPOSITIONS AND METHODS FOR IMPROVING MITOCHONDRIAL FUNCTION

(71) Applicant: YUVA BIOSCIENCES, INC., Birmingham, AL (US)

(72) Inventors: Keshav K. Singh, Birmingham, AL (US); Leopoldo Aguilera Aguirre, Birmingham, AL (US); Greg Schmergel, Lincoln, MA (US)

(21) Appl. No.: 18/282,719

(22) PCT Filed: Mar. 18, 2022
(86) PCT No.: PCT/US2022/020983

§ 371 Date: Sep. 18, 2023

Related U.S. Application Data
(60) Provisional application No. 63/162,927, filed on Mar. 18, 2021.

(51) Int. Cl. A61K 8/9789; A61K 8/11; A61K 36/185; A61Q 5/02; A61Q 19/00; A61Q 19/08

(52) U.S. Cl. CPC A61K 8/9789; A61K 8/11; A61K 36/185; A61Q 5/02; A61Q 19/00; A61Q 19/08

(57) ABSTRACT

Methods for treating or preventing mitochondrial dysfunction are disclosed. Methods for treating or preventing a disease or condition associated with mitochondrial dysfunction or a symptom thereof are disclosed. Methods for treating or preventing an aging-associated chronic disease or condition related to mitochondrial dysfunction or a symptom thereof are disclosed. Methods for improving or preventing a decrease in energy level and/or vitality are disclosed. The methods include administering an emblica extract or a compound constituent of an emblica extract, or fucus extract or a compound constituent of a fucus extract, or a chebula extract or a compound constituent of a chebula extract.

### CROSS-REFERENCE TO RELATED APPLICATIONS

This application is a U.S. national phase application and claims the benefit of priority under 35 U.S.C. § 371, of International (PCT) Patent Application Serial No. PCT/US2022/020983, filed on Mar. 18, 2022, which claims priority to of U.S. Provisional Application 63/162,927 filed on Mar. 18, 2021, which are incorporated herein by reference in their entireties.

### FIELD OF TECHNOLOGY

Aspects and embodiments disclosed herein relate to methods of treatment of diseases and conditions related to mitochondrial dysfunction. More specifically, aspects and embodiments disclosed herein relate to methods of treatment of diseases and conditions related to mitochondrial DNA depletion.

### BACKGROUND

Mitochondrial dysfunction, such as mitochondrial DNA (mtDNA) depletion, is involved in many diseases and conditions, such as mtDNA depletion syndromes, mitochondrial diseases, aging, aging-associated chronic diseases or conditions, reduced energy levels and vitality, characteristics of hair aging including hair loss and graying, characteristics of skin aging including skin wrinkles and senile lentigines, skin diseases and conditions, and other human pathologies. Fundamental questions about mitochondrial biology and mtDNA biology and their roles in such diseases and conditions remain mostly unsolved. Animal models capable of inducing mitochondrial dysfunction and/or modulating mtDNA copy number and/or concentration have been developed and provide a system for investigating mitochondrial pathology and its role in the disease process. There is a need for treatment methods for diseases and conditions related to mitochondrial dysfunction, such as mtDNA depletion.

### SUMMARY

In accordance with one aspect, there is provided a method of treating or preventing mitochondrial dysfunction in a subject. The method may comprise administering to the subject a composition comprising an effective amount of an emblica extract, or a pharmaceutically acceptable form thereof, or one or more compound constituent of an emblica extract or having a similarity score of at least 95% with a compound constituent of an emblica extract, or a pharmaceutically acceptable form thereof.

In accordance with one aspect, there is provided a method of treating or preventing a disease or condition associated with mitochondrial dysfunction or a symptom thereof in a subject. The method may comprise administering to the subject a composition comprising an effective amount of an emblica extract, or a pharmaceutically acceptable form thereof, or one or more compound constituent of an emblica extract or having a similarity score of at least 95% with a compound constituent of an emblica extract, or a pharmaceutically acceptable form thereof.

In accordance with one aspect, there is provided a method of treating or preventing an aging-associated chronic disease or condition related to mitochondrial dysfunction in a subject. The method may comprise administering to the subject a composition comprising an effective amount of an emblica extract, or a pharmaceutically acceptable form thereof, or one or more compound constituent of an emblica extract or having a similarity score of at least 95% with a compound constituent of an emblica extract, or a pharmaceutically acceptable form thereof.

In accordance with one aspect, there is provided a method of improving or preventing a decrease in energy level and/or vitality in a subject. The method may comprise administering to the subject a composition comprising an effective amount of an emblica extract, or a pharmaceutically acceptable form thereof, or one or more compound constituent of an emblica extract or having a similarity score of at least 95% with a compound constituent of an emblica extract, or a pharmaceutically acceptable form thereof.

In some embodiments, the emblica extract is derived from Emblica officinalis.

In some embodiments, the compound constituent of the emblica extract is a benzoic acid substituted with 1 to 5 hydroxy groups and optionally 1 to 3 O—(C₁-C₅ alkyl) or O—(C₁-C₅ alkenyl) groups, or a pharmaceutically acceptable form thereof, or a benzene substituted with —CH═CH—(CH₂)ₐ—C(O)OH, wherein a is 0 to 5, and 1 to 5 hydroxy groups, or a pharmaceutically acceptable form thereof, or a combination of the foregoing.

In some embodiments, the compound constituent of the emblica extract is gallic acid, vanillic acid, chlorogenic acid, caffeic acid, syringic acid, coumaric acid, quercetin, emblicanin A, emblicanin B, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

In accordance with one aspect, there is provided a method of treating or preventing mitochondrial dysfunction in a subject. The method may comprise administering to the subject a composition comprising an effective amount of a fucus extract, or a pharmaceutically acceptable form thereof, or one or more compound constituent of a fucus extract or having a similarity score of at least 95% with a compound constituent of a fucus extract, or a pharmaceutically acceptable form thereof.

In accordance with one aspect, there is provided a method of treating or preventing a disease or condition associated with mitochondrial dysfunction or a symptom thereof in a subject. The method may comprise administering to the subject a composition comprising an effective amount of a fucus extract, or a pharmaceutically acceptable form thereof, or one or more compound constituent of a fucus extract or having a similarity score of at least 95% with a compound constituent of a fucus extract, or a pharmaceutically acceptable form thereof.

In accordance with one aspect, there is provided a method of treating or preventing an aging-associated chronic disease or condition related to mitochondrial dysfunction in a subject. The method may comprise administering to the subject a composition comprising an effective amount of a fucus extract, or a pharmaceutically acceptable form thereof, or one or more compound constituent of a fucus extract or having a similarity score of at least 95% with a compound constituent of a fucus extract, or a pharmaceutically acceptable form thereof.

In accordance with one aspect, there is provided a method of improving or preventing a decrease in energy level and/or vitality in a subject. The method may comprise administering to the subject a composition comprising an effective amount of a fucus extract, or a pharmaceutically acceptable form thereof, or one or more compound constituent of a fucus extract or having a similarity score of at least 95% with a compound constituent of a fucus extract, or a pharmaceutically acceptable form thereof.

In some embodiments, the fucus extract is derived from Fucus vesiculosus, Fucus serratus, Fucus, spiralis, or Fucus guiryi.

In some embodiments, the compound constituent of the fucus extract is a benzoic acid substituted with 1 to 5 hydroxy groups and optionally 1 to 3 O—(C₁-C₅ alkyl) or O—(C₁-C₅ alkenyl) groups, or a pharmaceutically acceptable form thereof, or a benzene substituted with —CH═CH—(CH₂)ₐ—C(O)OH, wherein a is 0 to 5, and 1 to 5 hydroxy groups, or a pharmaceutically acceptable form thereof, or a combination of the foregoing.

In some embodiments, the compound constituent of the fucus extract is gallic acid, vanillic acid, chlorogenic acid, caffeic acid, syringic acid, coumaric acid, quercetin, fucoidan, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

In accordance with one aspect, there is provided a method of treating or preventing mitochondrial dysfunction in a subject, the method comprising administering to the subject a composition comprising an effective amount of a chebula extract, or a pharmaceutically acceptable form thereof, or one or more compound constituent of a chebula extract or having a similarity score of at least 95% with a compound constituent of a chebula extract, or a pharmaceutically acceptable form thereof.

In accordance with one aspect, there is provided a method of treating or preventing a disease or condition associated with mitochondrial dysfunction or a symptom thereof in a subject, the method comprising administering to the subject a composition comprising an effective amount of a chebula extract, or a pharmaceutically acceptable form thereof, or one or more compound constituent of a chebula extract or having a similarity score of at least 95% with a compound constituent of a chebula extract, or a pharmaceutically acceptable form thereof.

In accordance with one aspect, there is provided a method of treating or preventing an aging-associated chronic disease or condition related to mitochondrial dysfunction in a subject, the method comprising administering to the subject a composition comprising an effective amount of a chebula extract, or a pharmaceutically acceptable form thereof, or one or more compound constituent of a chebula extract or having a similarity score of at least 95% with a compound constituent of a chebula extract, or a pharmaceutically acceptable form thereof.

In accordance with one aspect, there is provided a method of improving or preventing a decrease in energy level and/or vitality in a subject, the method comprising administering to the subject a composition comprising an effective amount of a chebula extract, or a pharmaceutically acceptable form thereof, or one or more compound constituent of a chebula extract or having a similarity score of at least 95% with a compound constituent of a chebula extract, or a pharmaceutically acceptable form thereof.

In some embodiments, the chebula extract is derived from Terminilia chebula, Terminalia arborea, or Lumnitzera racemose.

In some embodiments, the compound constituent of the chebula extract is a benzoic acid substituted with 1 to 5 hydroxy groups and optionally 1 to 3 O—(C₁-C₅ alkyl) or O—(C₁-C₅ alkenyl) groups, or a pharmaceutically acceptable form thereof, or a benzene substituted with —CH═CH—(CH₂)ₐ—C(O)OH, wherein a is 0 to 5, and 1 to 5 hydroxy groups, or a pharmaceutically acceptable form thereof, or a combination of the foregoing.

In some embodiments, the compound constituent of the chebula extract is gallic acid, vanillic acid, chlorogenic acid, caffeic acid, syringic acid, coumaric acid, quercetin, tannic acid, punigluconin, pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

In some embodiments, the composition comprises two or more of:
an effective amount of an emblica extract, or a pharmaceutically acceptable form thereof, or a compound constituent of an emblica extract or having a similarity score of at least 95% with a compound constituent of an emblica extract, or a pharmaceutically acceptable form thereof;
an effective amount of a fucus extract, or a pharmaceutically acceptable form thereof, or a compound constituent of a fucus extract or having a similarity score of at least 95% with a compound constituent of a fucus extract, or a pharmaceutically acceptable form thereof; and
an effective amount of a chebula extract, or a pharmaceutically acceptable form thereof, or a compound constituent of a chebula extract or having a similarity score of at least 95% with a compound constituent of a chebula extract, or a pharmaceutically acceptable form thereof.

In some embodiments, the method may comprise, administering to the subject two or more of:
an effective amount of an emblica extract, or a pharmaceutically acceptable form thereof, or a compound constituent of an emblica extract or having a similarity score of at least 95% with a compound constituent of an emblica extract, or a pharmaceutically acceptable form thereof;
an effective amount of a fucus extract, or a pharmaceutically acceptable form thereof, or a compound constituent of contained a fucus extract or having a similarity score of at least 95% with a compound constituent of an fucus extract, or a pharmaceutically acceptable form thereof; and
an effective amount of a chebula extract, or a pharmaceutically acceptable form thereof, or a compound constituent of a chebula extract or having a similarity score of at least 95% with a compound constituent of a chebula extract, or a pharmaceutically acceptable form thereof.

In some embodiments, the method further comprises administering to the subject a second or subsequent amount of the emblica extract, or a pharmaceutically acceptable form thereof, or a compound constituent of an emblica extract or having a similarity score of at least 95% with a compound constituent of an emblica extract, or a pharmaceutically acceptable form thereof.

In some embodiments, the method further comprises administering to the subject a second or subsequent amount of the fucus extract, or a pharmaceutically acceptable form thereof, or a compound constituent of a fucus extract or having a similarity score of at least 95% with a compound constituent of a fucus extract, or a pharmaceutically acceptable form thereof.

In some embodiments, the method further comprises administering to the subject a second or subsequent amount of the chebula extract, or a pharmaceutically acceptable form thereof, or a compound constituent of a chebula extract or having a similarity score of at least 95% with a compound constituent of a chebula extract, or a pharmaceutically acceptable form thereof.

In some embodiments, the second or subsequent amount is administered as a second dose of the same formulation.

In some embodiments, the second or subsequent amount is administered in another formulation.

In some embodiments, the composition is fortified with one or more compound constituent of an emblica extract or having a similarity score of at least 95% with a compound constituent of an emblica extract, or a pharmaceutically acceptable form thereof.

In some embodiments, the composition is fortified with one or more compound constituent of a fucus extract or having a similarity score of at least 95% with a compound constituent of a fucus extract, or a pharmaceutically acceptable form thereof.

In some embodiments, the one or more compound constituent of an emblica extract or having a similarity score of at least 95% with a compound constituent of an emblica extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the one or more compound constituent of the fucus extract or having a similarity score of at least 95% with a compound constituent of a fucus extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the effective amount is a therapeutically effective amount.

In some embodiments, the treatment or prevention involves inducing mitochondrial biogenesis and/or improving mitochondrial function.

In some embodiments, the effective amount or therapeutically effective amount is sufficient to induce mitochondrial biogenesis.

In some embodiments, administration increases expression of at least one protein selected from PGC-1a, TFAM, NRF-1, and COX II.

In some embodiments, the composition is administered topically.

In some embodiments, the composition is administered parenterally, e.g., intravenously, intraperitoneally, or intramuscularly.

In some embodiments, the composition is administered enterally.

In some embodiments, the composition is formulated as a topical solution, oil, cream, emulsion, or gel.

In some embodiments, the composition is formulated as a shampoo, conditioner, spray, cream, gel, balm, body wash, soap, lotion, or make-up.

In some embodiments, the composition is formulated as a parenteral liquid solution.

In some embodiments, the composition is formulated as an enteral capsule or tablet, or dietary supplement or food, e.g., food, food supplement, medical food, food additive, nutraceutical, or drink.

In some embodiments, the composition is administered locally.

In some embodiments, the composition is administered systemically.

In some embodiments, the composition is formulated for immediate release.

In some embodiments, the composition is formulated for extended release, e.g., controlled or sustained release.

In some embodiments, the composition comprises a nanoparticle-based delivery carrier.

In some embodiments, the composition comprises a skin penetration enhancer or is administered in combination with a skin penetration enhancer, e.g., a chemical skin penetration enhancer or a physical skin penetration enhancer.

In some embodiments, the composition comprises a mitochondria-targeting agent or a delivery carrier functionalized with a mitochondria-targeting agent.

In some embodiments, the disease or condition associated with mitochondrial dysfunction or symptom thereof is a cardiovascular disease, diabetes, cancer, neurological disorder, or skin disease or condition.

In some embodiments, the skin disease or condition is skin wrinkles, change in skin pigmentation, senile lentigines, or a disease or condition of sebaceous glands.

In some embodiments, the skin disease or condition is a scalp or hair disease or condition, e.g., hair loss, hair thinning, or change in hair pigmentation.

In some embodiments, the compound constituent of an emblica extract was derived from, purified from, or isolated from the emblica extract.

In some embodiments, the compound constituent of an emblica extract was derived from, purified from, or isolated from a source other than the emblica extract.

In some embodiments, the compound constituent of an emblica extract was synthesized.

In some embodiments, the compound constituent of a fucus extract was derived from, purified from, or isolated from the emblica extract.

In some embodiments, the compound constituent of a fucus extract was derived from, purified from, or isolated from a source other than the emblica extract.

In some embodiments, the compound constituent of a fucus extract was synthesized.

In some embodiments, the compound constituent of a chebula extract was derived from, purified from, or isolated from the emblica extract.

In some embodiments, the compound constituent of a chebula extract was derived from, purified from, or isolated from a source other than the emblica extract.

In some embodiments, the compound constituent of a chebula extract was synthesized.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A shows quantification of immunofluorescence analysis of OXPHOS complex IV (COXII) in relative fluorescence units (RFUs) in paraffin dorsal skin sections control and mtDNA-depleter mice after dox induction and 16 weeks of topical emblica extract treatment or control treatment. Data are mean±SD (n=3); *P<0.05;

FIG. 1B shows RT-PCR analysis of dorsal skin RNA for mtDNA encoded genes from control and mtDNA-depleter mice after dox induction and 16 weeks of topical emblica extract treatment or control treatment;

FIG. 1C shows quantification of mtDNA content (mean±s.e.m; *P<0.05, Student's t test) in dorsal skin samples from control (n=3) and mtDNA-depleter (n=3) mice after dox induction and 16 weeks of topical emblica extract treatment or control treatment application;

FIG. 1D shows quantitative analysis of inflammatory cells in the skin sections from control and mtDNA-depleter mice after dox induction and 16 weeks of topical emblica extract treatment or control treatment (mean±SD; *P≤0.05; **P≤0.01; ***P≤0.001 Student's t test);

FIG. 2A shows representative pictures of: (i) control mice after dox induction; (ii) mtDNA-depleter mice after dox-induction; (iii) control mice after dox induction and 51 days of treatment with fucus extract; and (iv) mtDNA-depleter mice after dox induction and 51 days of treatment with fucus extract (preventive intervention) (n=3 for each group);

FIG. 2B shows quantification of mtDNA content (mean±s.e.m; *P<0.05, Student's t test) in dorsal skin samples from control (n=3) and mtDNA-depleter (n=3) mice after dox induction and 16 weeks of topical fucus extract treatment or control treatment application;

FIG. 3 shows schematics of emblica extract preventive and therapeutic in vivo experiments;

FIG. 4A is a graph showing induced expression of mitochondrial complex IV subunit 2 (COXII) by various compositions, according to one embodiment;

FIG. 4B is a graph showing induced expression of mitochondrial transcription factor A (TFAM) by various compositions, according to one embodiment;

FIG. 4C is a graph showing induced expression of peroxisome proliferator-activated receptor gamma coactivator 1-alpha (PGC-1a) by various compositions, according to one embodiment;

FIG. 5 is an image of an electrophoresis gel showing protein levels of the mitochondrial biogenesis markers COXII, TFAM, and PGC-1a induced by various compositions, according to one embodiment;

FIG. 6A is a graph showing percentage questionnaire answers provided by participants receiving a low dose of the compositions disclosed herein during an in vivo skin wrinkle study;

FIG. 6B is a graph showing percentage questionnaire answers provided by participants receiving a high dose of the compositions disclosed herein during the in vivo skin wrinkle study;

FIGS. 7A-7B are graphs showing COXII expression induced by administration of an emblica extract in vitro at 6 hours to 96 hours after administration;

FIGS. 8A-8B are graphs showing TFAM expression induced by administration of an emblica extract in vitro at 6 hours to 96 hours after administration;

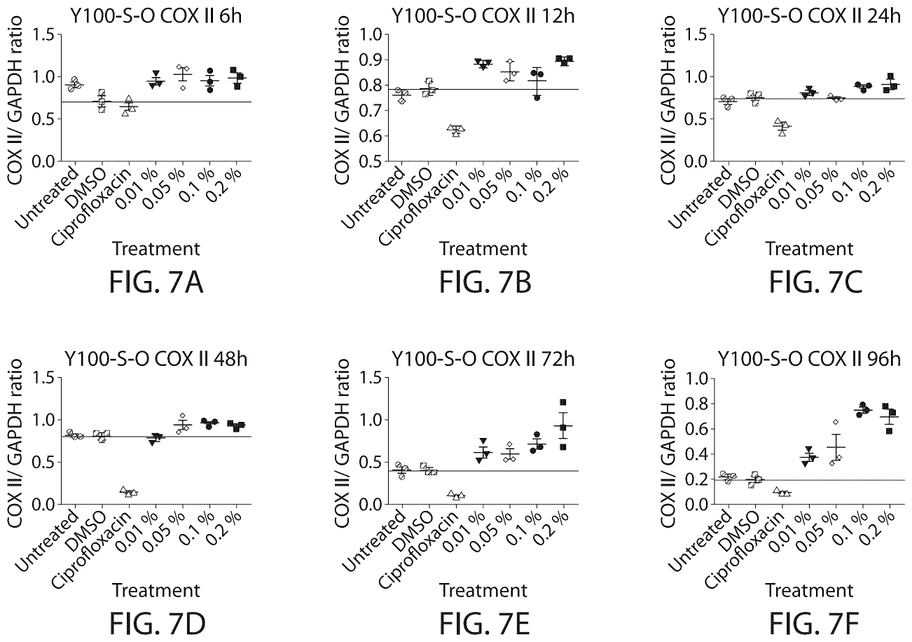

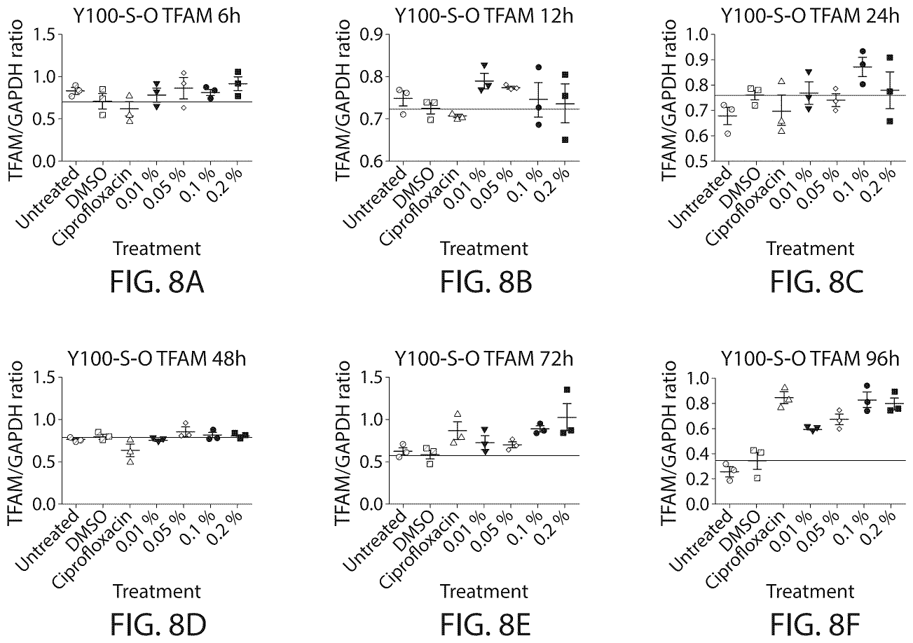

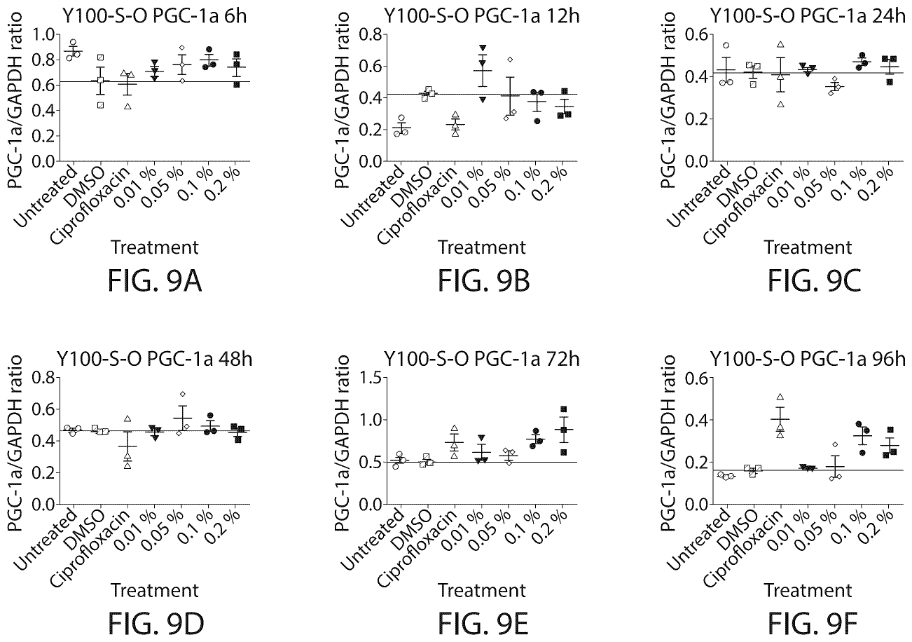

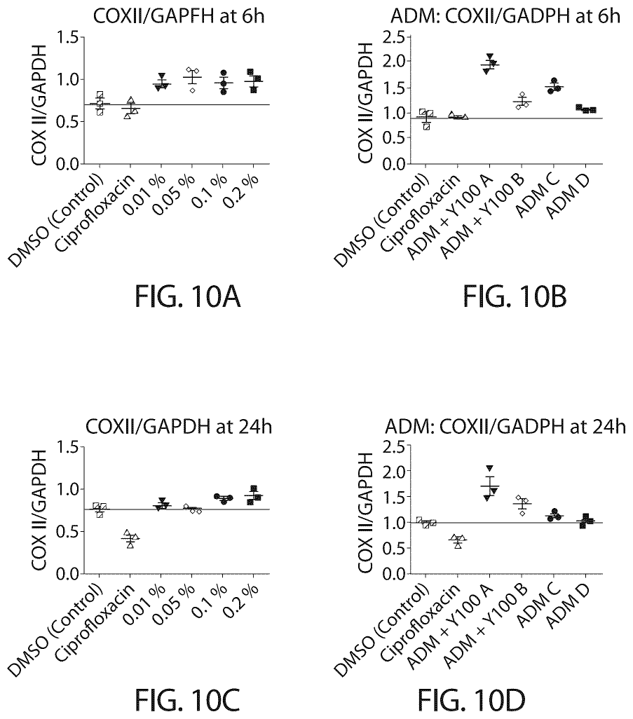

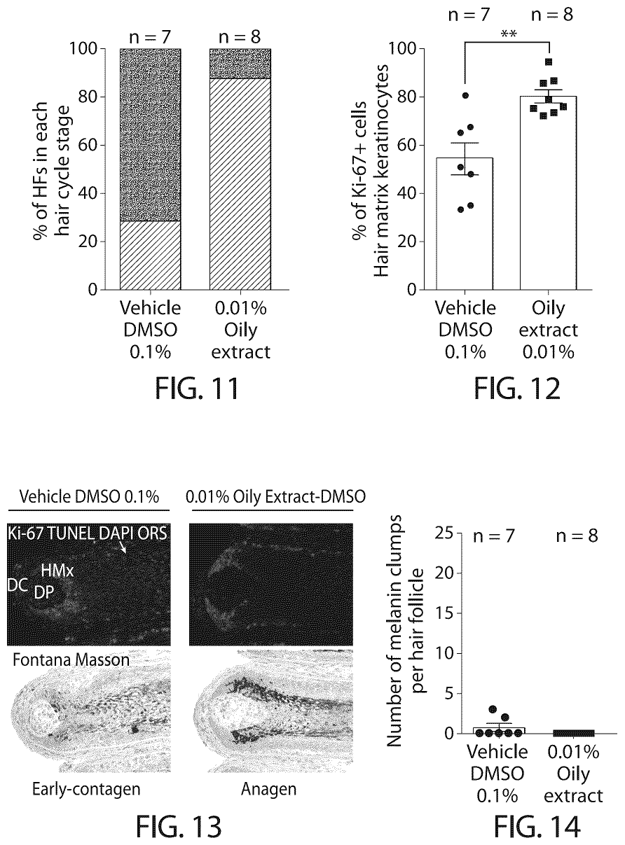

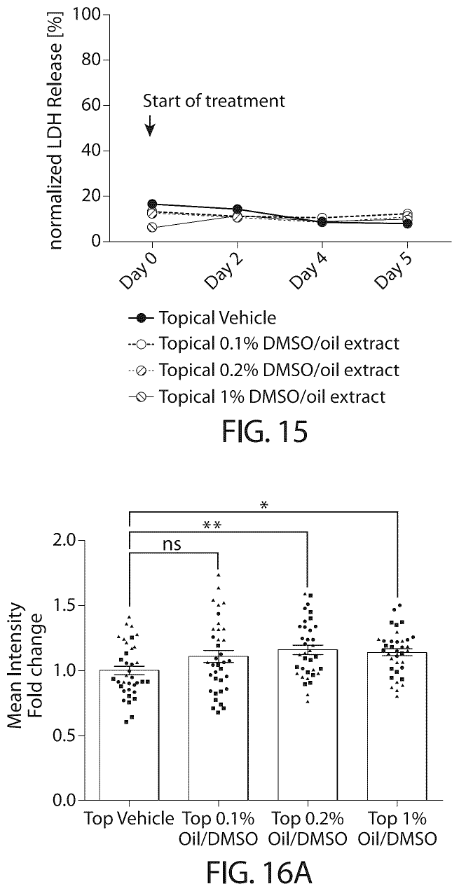

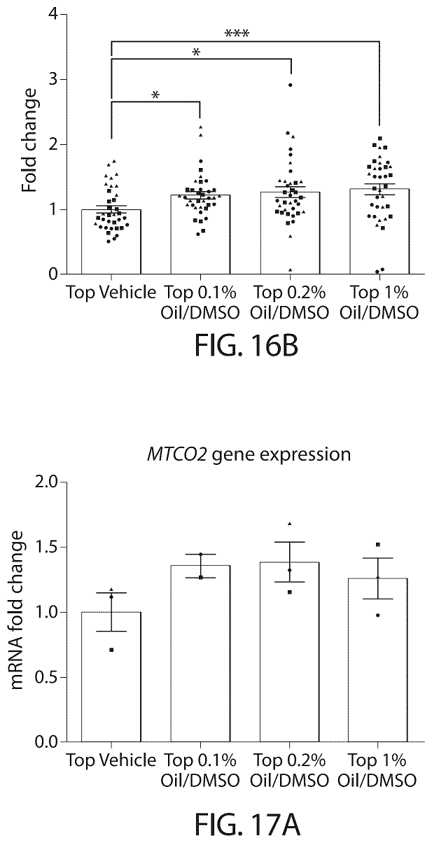

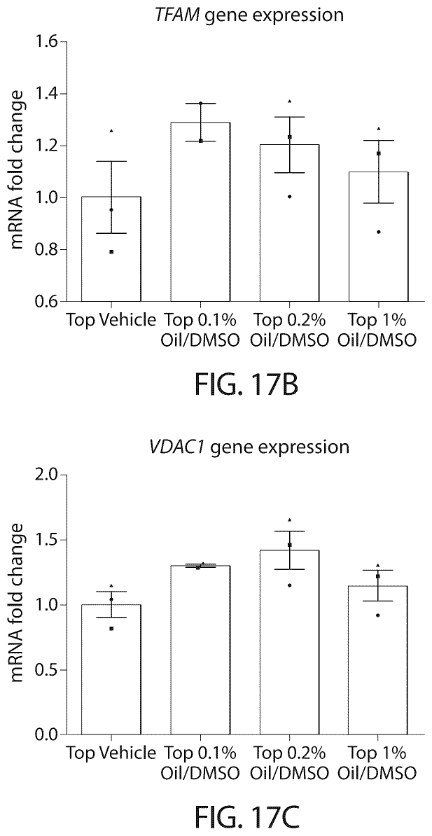

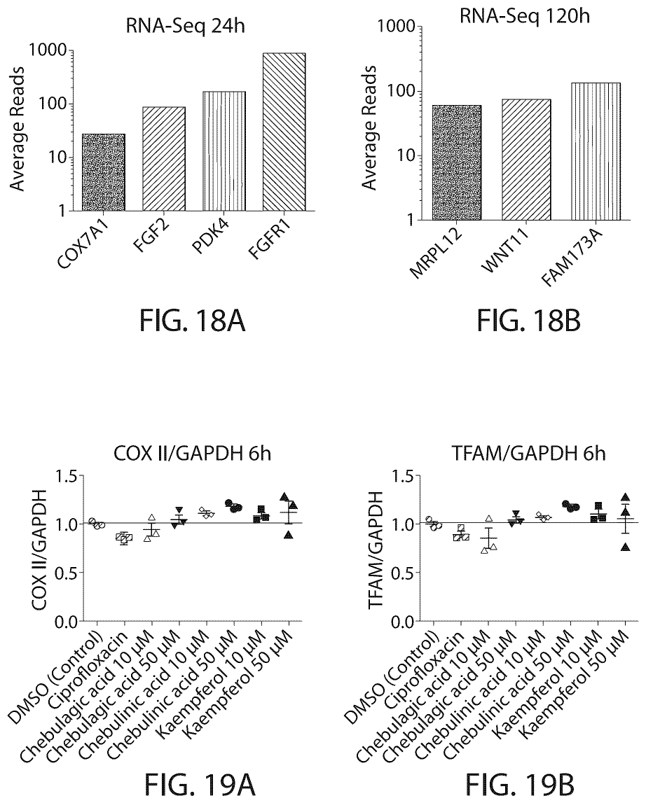

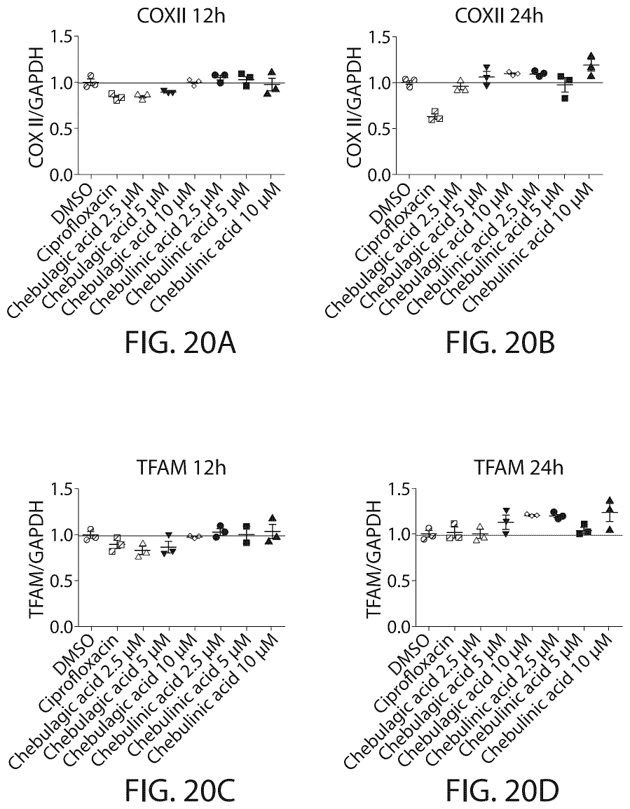

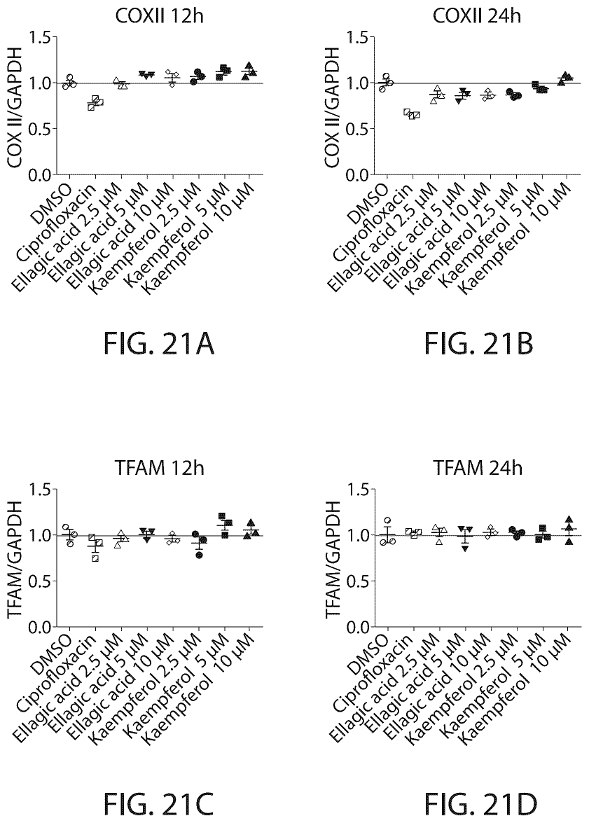

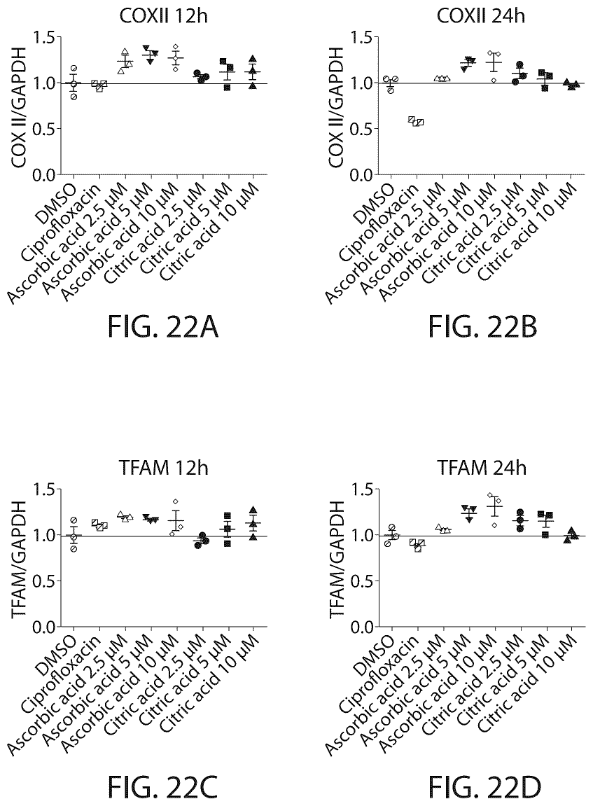

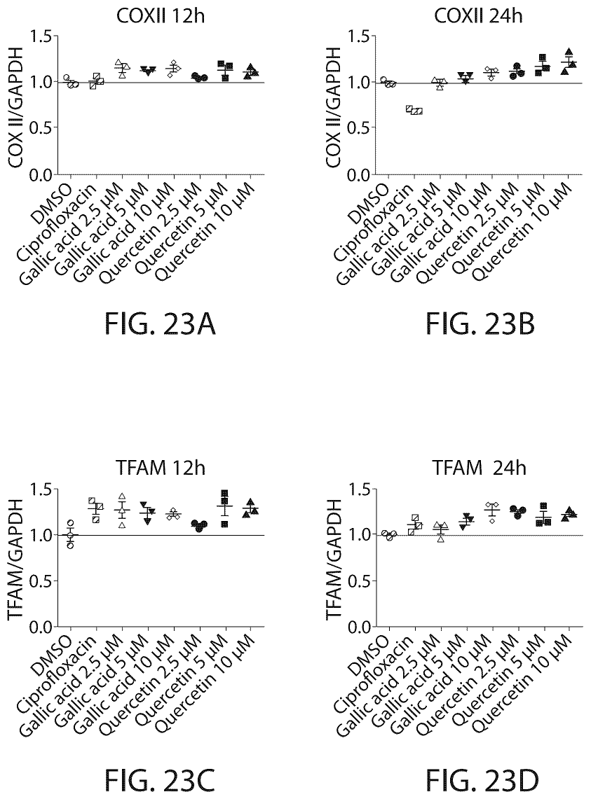

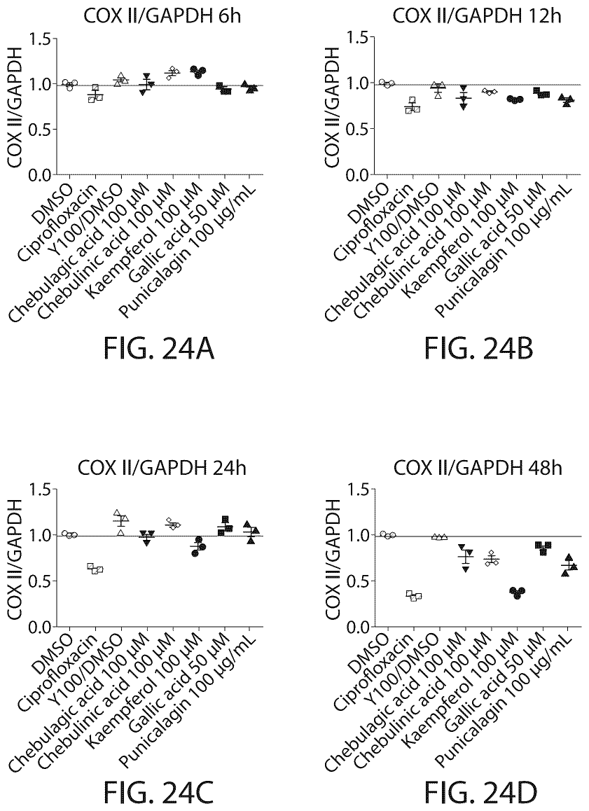

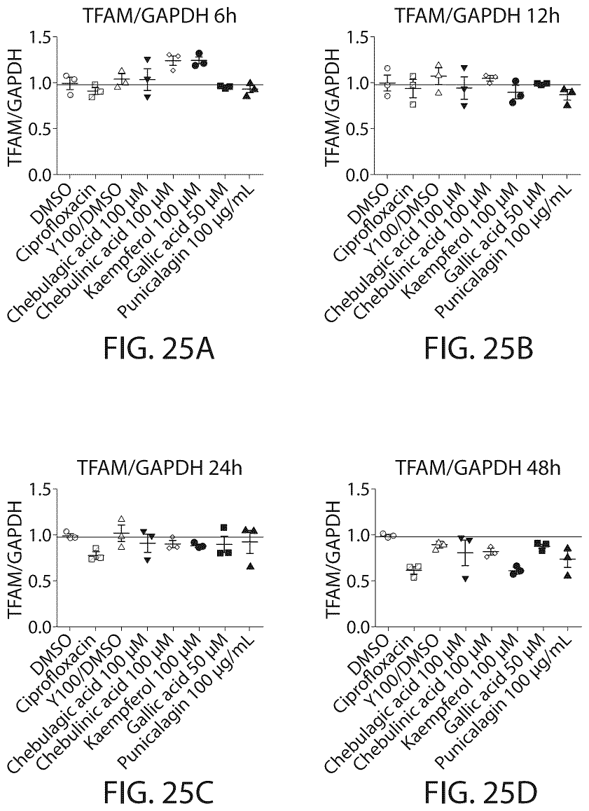

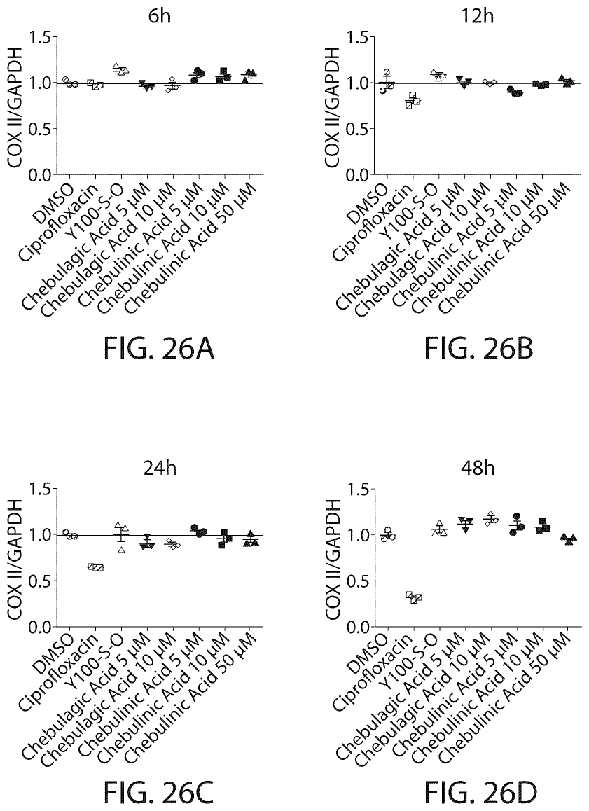

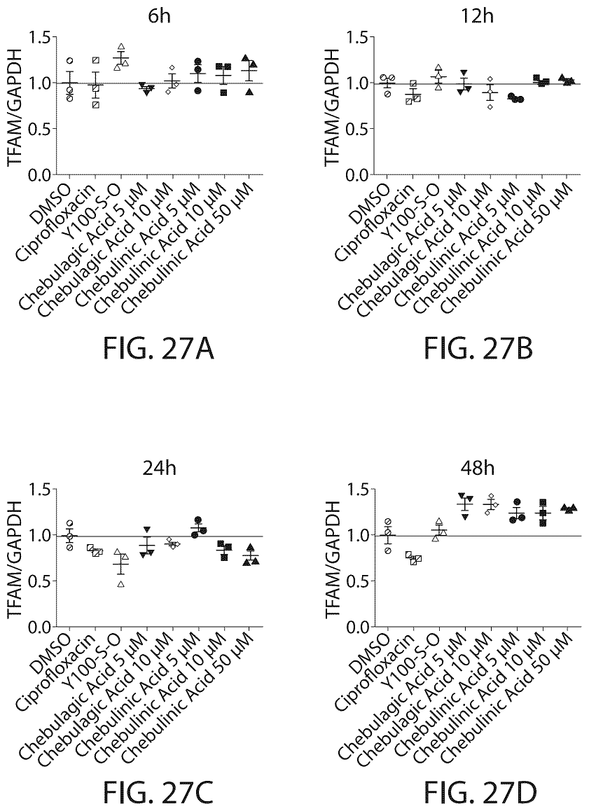

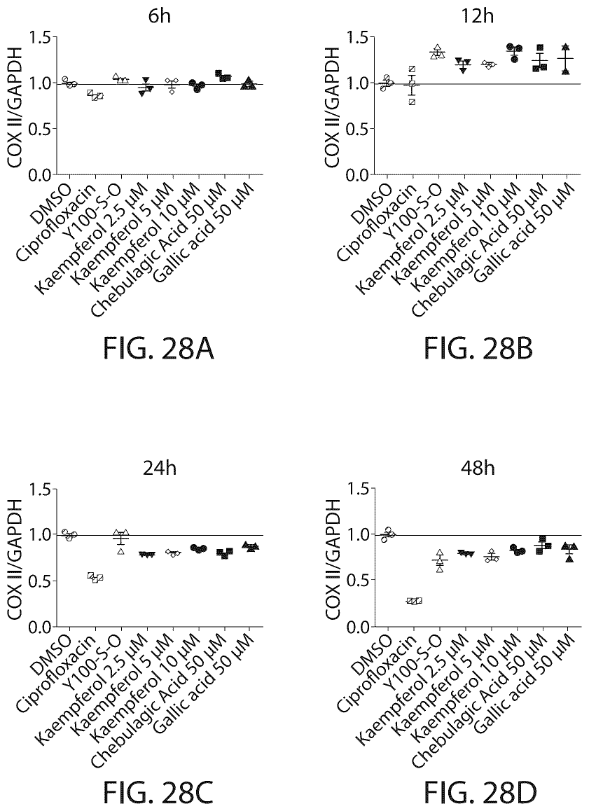

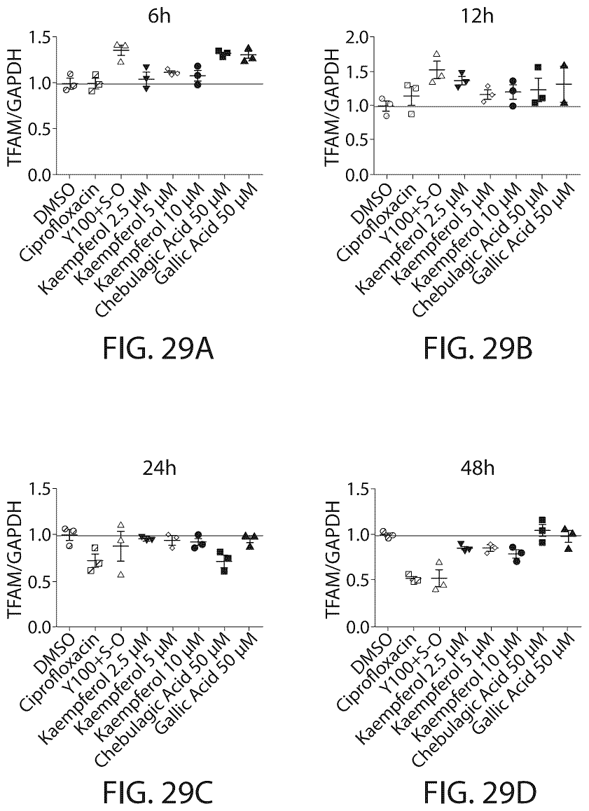

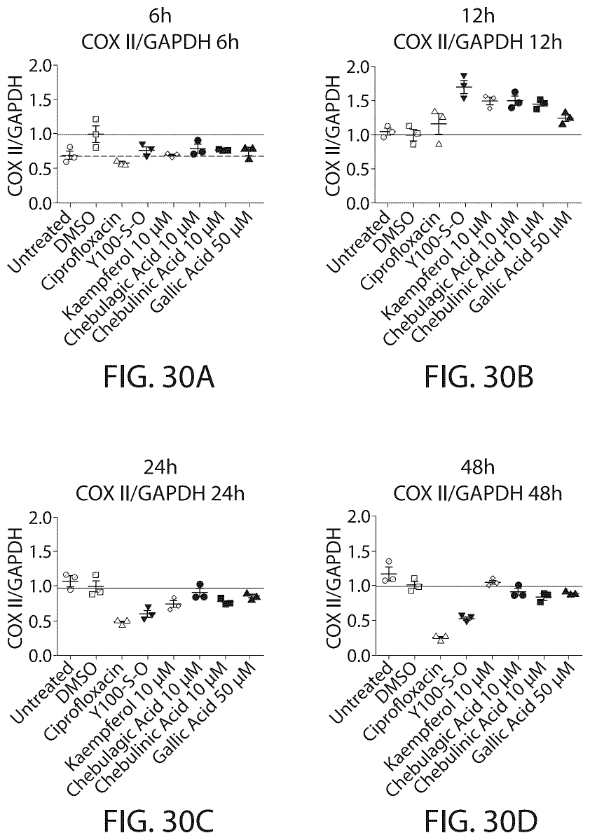

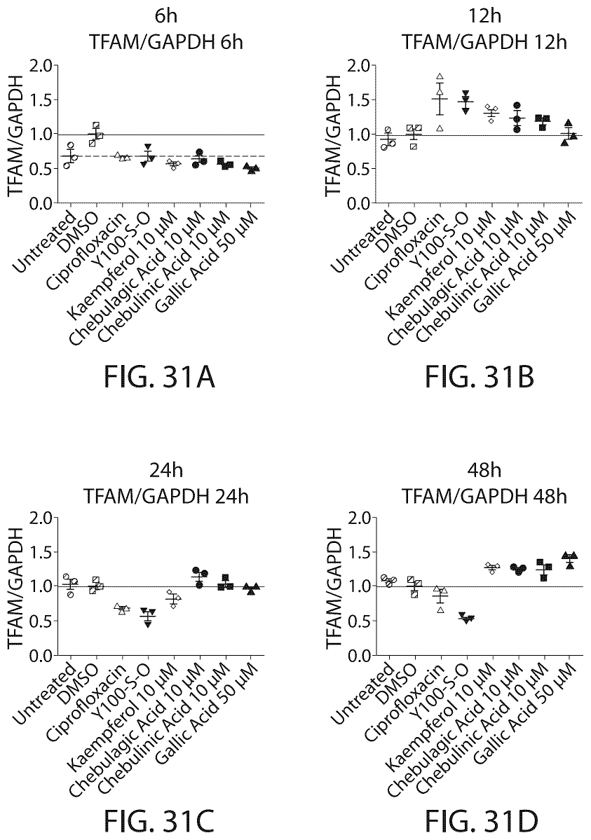

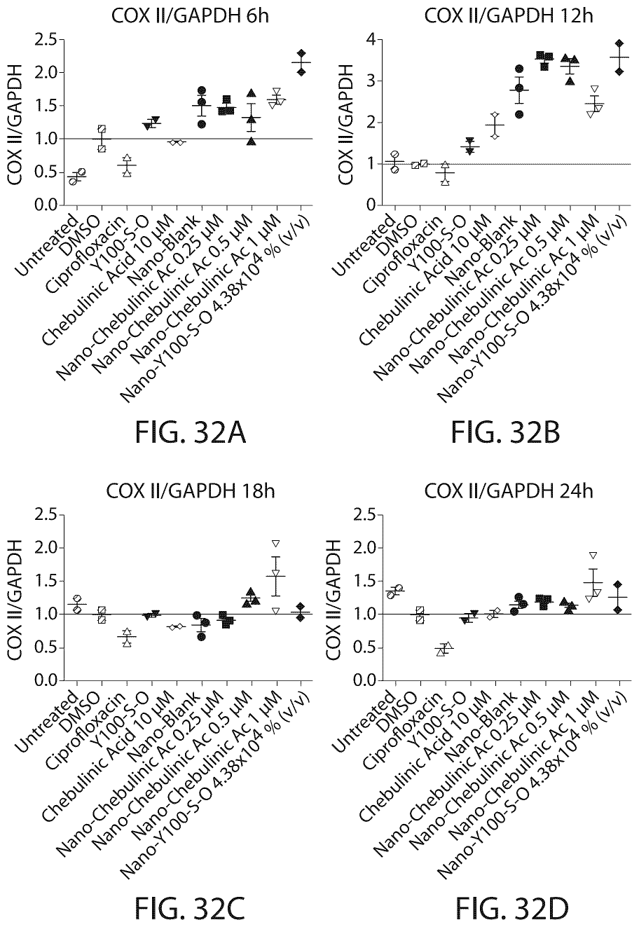

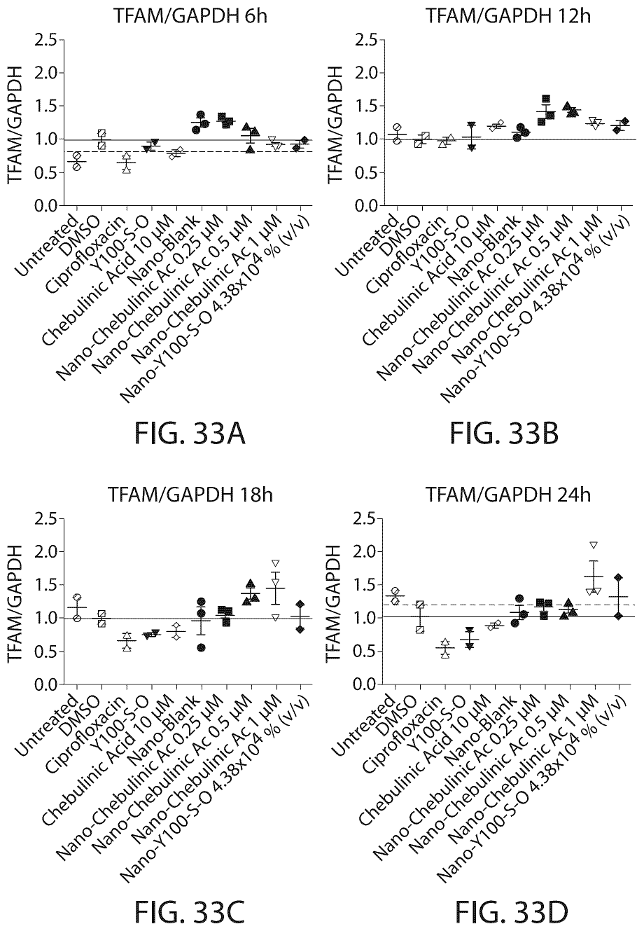

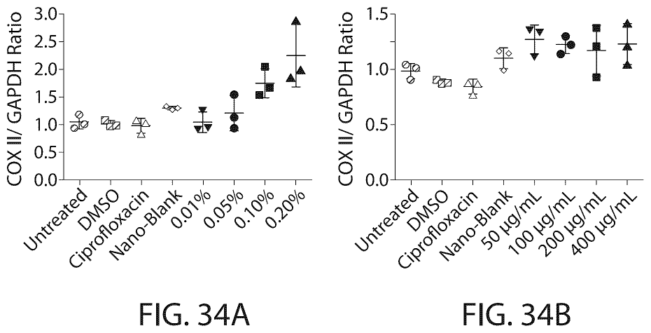

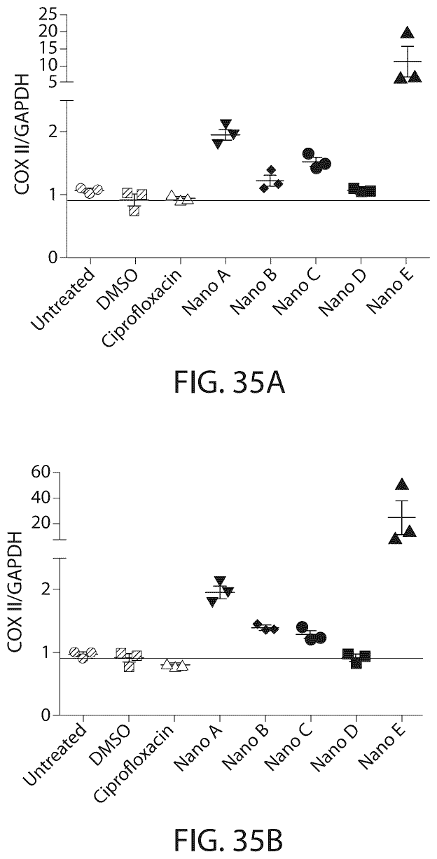

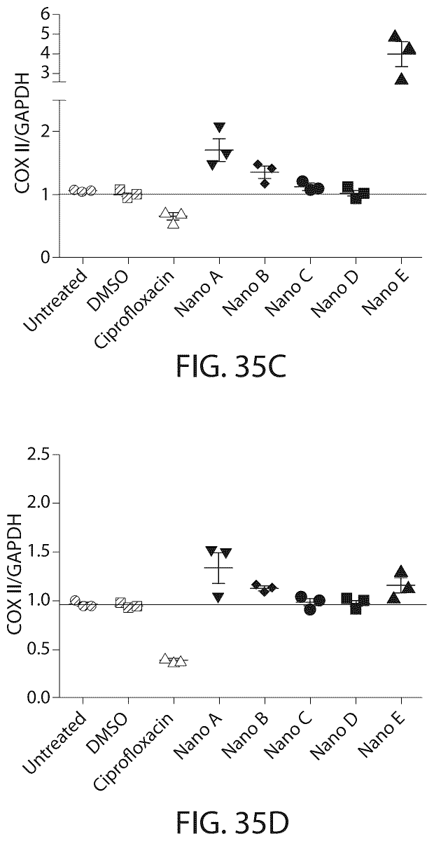

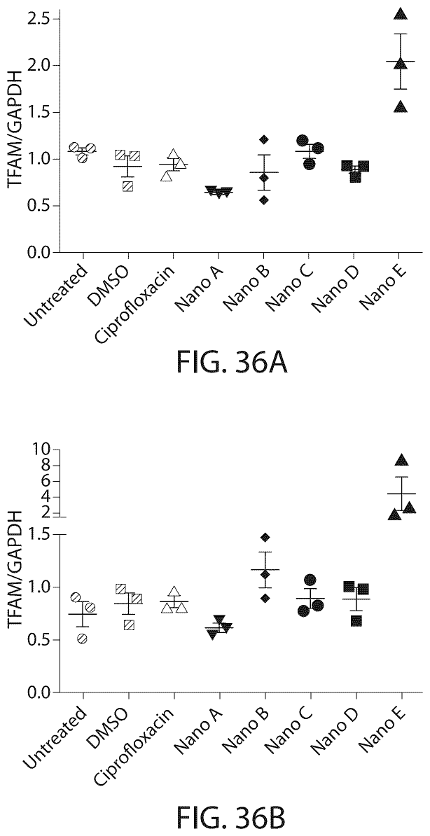

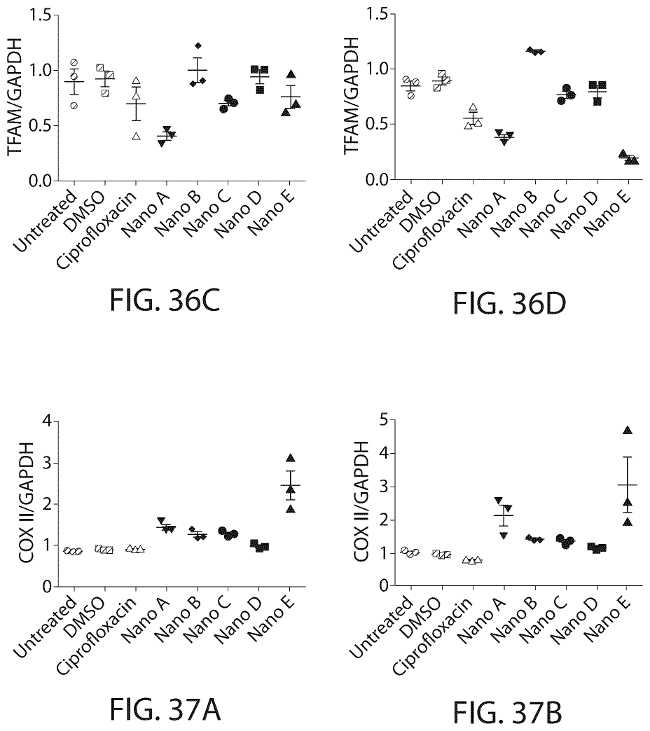

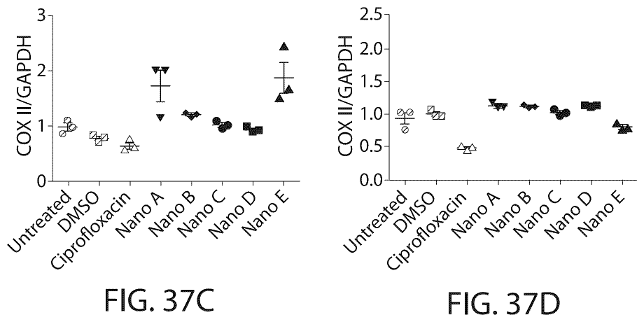

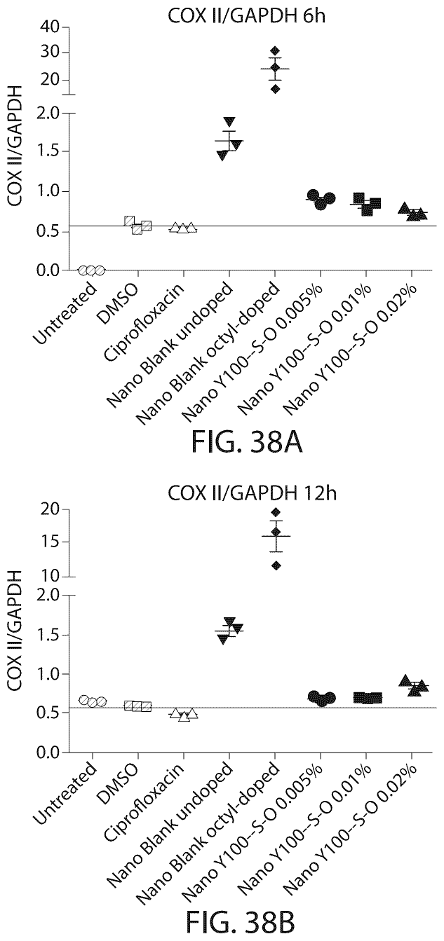

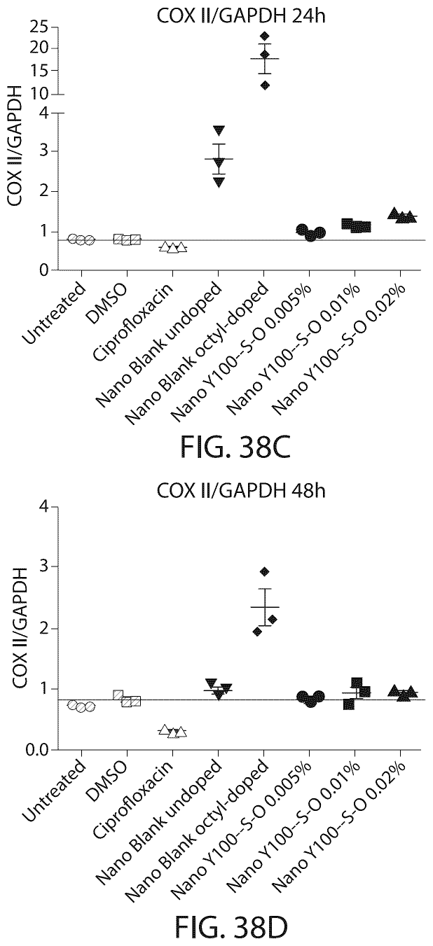

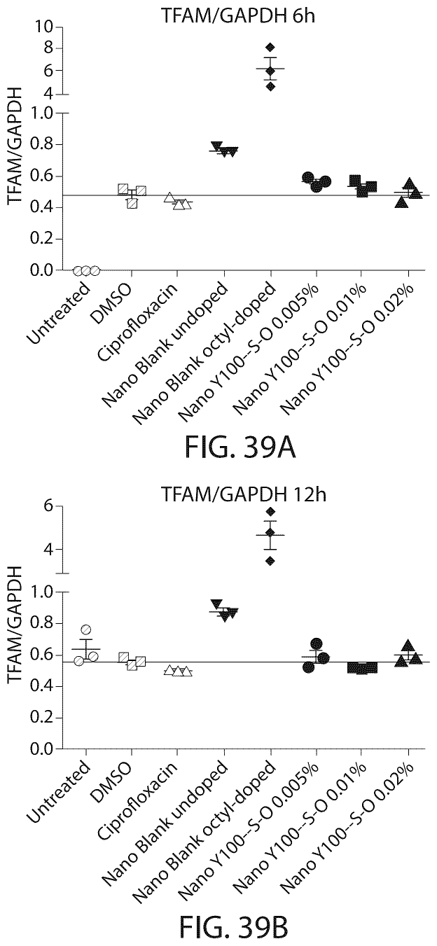

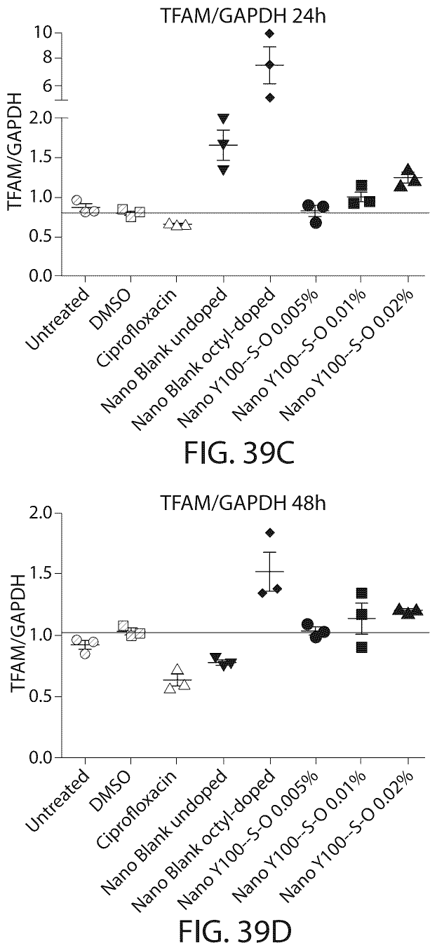

FIGS. 7A-7B are graphs showing PCG-1a expression induced by administration of an emblica extract in vitro at 6 hours to 96 hours after administration;

FIGS. 10-10D are graphs showing COXII expression induced by administration of an emblica extract in vitro, optionally with a nanoparticle delivery carrier, at 6 hours and 24 hours after administration;

FIG. 11 is a graph showing microscopic human hair cycle staging after ex vivo administration of an emblica extract, according to one embodiment;

FIG. 12 is a graph showing human hair matrix keratinocyte proliferation after ex vivo administration of an emblica extract, according to one embodiment;

FIG. 13 includes images showing immunohistochemical analysis of keratinocyte proliferation after ex vivo administration to human hair of an emblica extract, according to one embodiment;

FIG. 14 is a graph showing melanin clumping after ex vivo administration of an emblica extract to human hair, according to one embodiment;

FIG. 15 is a graph showing LDH release after ex vivo administration of an emblica extract to human skin, according to one embodiment;

FIGS. 16A-16B are graphs showing expression of COL1A1 gene at 24 hours and 5 days after ex vivo administration of an emblica extract to human skin, according to one embodiment;

FIGS. 17A-17C are graphs showing expression of MTCO2, TFAM, and VDAC genes, respectively, at 24 hours after ex vivo administration of an emblica extract to human skin, according to one embodiment;

FIGS. 18A-18B are graphs showing expression of FGF2, FGFR1, COX7A1, PDK4, FAM173A, MRPL12, and WNT11 genes at 24 or 120 hours after ex vivo administration of an emblica extract, according to one embodiment;

FIGS. 19A-19B include graphs showing expression of COXII and TFAM at 6 hours after in vitro administration of a constituent of emblica extract, according to one embodiment;

FIGS. 20A-20D include graphs showing expression of COXII and TFAM at 12 hours and 24 hours after in vitro administration of a constituent of emblica extract, according to one embodiment;

FIGS. 21A-21D include graphs showing expression of COXII and TFAM at 12 hours and 24 hours after in vitro administration of a constituent of emblica extract, according to one embodiment;

FIGS. 22A-22D include graphs showing expression of COXII and TFAM at 12 hours and 24 hours after in vitro administration of a constituent of emblica extract, according to one embodiment;

FIGS. 23A-23D include graphs showing expression of COXII and TFAM at 12 hours and 24 hours after in vitro administration of a constituent of emblica extract, according to one embodiment;

FIGS. 24A-24D include graphs showing expression of COXII at 6, 12, 24, and 48 hours after in vitro administration of a constituent of emblica extract, according to one embodiment;

FIGS. 25A-25D include graphs showing expression of TFAM at 6, 12, 24, and 48 hours after in vitro administration of a constituent of emblica extract, according to one embodiment;

FIGS. 26A-26D include graphs showing expression of COXII at 6, 12, 24, and 48 hours after in vitro administration of a constituent of emblica extract, according to one embodiment;

FIGS. 27A-27D include graphs showing expression of TFAM at 6, 12, 24, and 48 hours after in vitro administration of a constituent of emblica extract, according to one embodiment;

FIGS. 28A-28D include graphs showing expression of COXII at 6, 12, 24, and 48 hours after in vitro administration of a constituent of emblica extract, according to one embodiment;

FIGS. 29A-29D include graphs showing expression of TFAM at 6, 12, 24, and 48 hours after in vitro administration of a constituent of emblica extract, according to one embodiment;

FIGS. 30A-30D include graphs showing expression of COXII at 6, 12, 24, and 48 hours after in vitro administration of a constituent of emblica extract, according to one embodiment;

FIGS. 31A-31D include graphs showing expression of TFAM at 6, 12, 24, and 48 hours after in vitro administration of a constituent of emblica extract, according to one embodiment;

FIGS. 32A-32D include graphs showing expression of COXII at 6, 12, 24, and 48 hours after in vitro administration of a chebulinic acid, optionally encapsulated in a nanoparticle carrier, according to one embodiment;

FIGS. 33A-33D include graphs showing expression of TFAM at 6, 12, 24, and 48 hours after in vitro administration of chebulinic acid, optionally encapsulated in a nanoparticle carrier, according to one embodiment;

FIGS. 34A-34B include graphs showing expression of COXII at 6 hours after in vitro administration of emblica extract encapsulated in a nanoparticle carrier, according to one embodiment;

FIGS. 35A-35D include graphs showing expression of COXII at 6, 12, 24, and 48 hours after in vitro administration of emblica extract encapsulated in a nanoparticle carrier, according to one embodiment;

FIGS. 36A-36D include graphs showing expression of TFAM at 6, 12, 24, and 48 hours after in vitro administration of emblica extract encapsulated in a nanoparticle carrier, according to one embodiment;

FIGS. 37A-37D include graphs showing expression of COXII at 6, 12, 24, and 48 hours after in vitro administration of emblica extract encapsulated in a nanoparticle carrier, according to one embodiment;

FIGS. 38A-38D include graphs showing expression of COXII at 6, 12, 24, and 48 hours after in vitro administration of emblica extract encapsulated in a nanoparticle carrier, according to one embodiment; and

FIGS. 39A-39D include graphs showing expression of TFAM at 6, 12, 24, and 48 hours after in vitro administration of emblica extract encapsulated in a nanoparticle carrier, according to one embodiment.

### DETAILED DESCRIPTION

Mitochondrial dysfunction is associated with many mitochondrial diseases, many of which are the result of dysfunctional mitochondrial oxidative phosphorylation (OXPHOS). Mitochondrial OXPHOS accounts for the generation of most of the cellular adenosine triphosphate (ATP) in a cell. The OXPHOS function largely depends on the coordinated expression of proteins encoded by both nuclear and mitochondrial genomes. The human mitochondrial genome encodes for 13 polypeptides of the OXPHOS system, and the nuclear genome encodes the remaining more than 85 polypeptides required for the assembly of OXPHOS system. mtDNA depletion impairs OXPHOS and leads to mtDNA depletion syndromes (Alberio, et al., Mitochondrion 7, 6-12, 2007; Ryan, Met al., Annu. Rev. Biochem. 76, 701-722, 2007). The mtDNA depletion syndromes are a heterogeneous group of disorders, characterized by low mtDNA levels in specific tissues. In different target organs, mtDNA depletion leads to specific pathological changes (Tuppen, et al., Biochim. Biophys. Acta 1797, 113-128, 201)). mtDNA depletion syndromes result from the genetic defects in the nuclear-encoded genes that participate in mtDNA replication, and mitochondrial nucleotide metabolism and nucleotide salvage pathway (Alberio, et al., Mitochondrion 7, 6-12, 2007). mtDNA depletion is also implicated in other human diseases and conditions, such as, but not limited to, mtDNA depletion syndromes, mitochondrial diseases, aging, aging-associated chronic diseases or conditions, reduced energy levels and vitality, characteristics of hair aging including hair loss and graying, characteristics of skin aging including skin wrinkles and senile lentigines, skin diseases and conditions, and other human pathologies.

A general decline in mitochondrial function has been extensively reported during aging. Furthermore, mitochondrial dysfunction is known to be a driving force underlying age-related human diseases. A mouse that carries a specific mtDNA mutation has been shown to present signs of premature aging (i.e., a mtDNA depleter mouse). In addition to mutations in mtDNA, studies also suggest a decrease in mtDNA content and mitochondrial copy number with age. Low mtDNA copy number is linked to frailty and, for a multiethnic population, is a predictor of all-cause mortality. A recent study revealed that humans on an average lose about four copies of mtDNA every ten years. This study also identified an association of decrease in mtDNA copy number with age-related physiological parameters.

Accumulating evidence suggests a strong link between mitochondrial dysfunction, mitochondrial diseases, aging, and aging-associated diseases. Notably, increased somatic mtDNA mutations and decline in mitochondrial functions have been extensively reported during human aging. Studies also suggest a decrease in mtDNA content and mitochondrial number with age.

### Definitions

As used herein, the term “carrier” refers to a diluent or vehicle with which a compound is administered. The carrier may be a pharmaceutically acceptable carrier. The carrier may be a cosmetically acceptable carrier. Examples of suitable pharmaceutical carriers are described in “Remington's Pharmaceutical Sciences” by E. W. Martin.

As used herein, the term “corresponding aspartic acid” means an aspartic acid (D) residue that is mutated to an alanine (A) residue in a POLG1 amino acid sequence that is the equivalent of the aspartic acid at position 1135 of the human POLG1 sequence. In a particular embodiment, the “corresponding aspartic acid” is flanked on the amino terminus side by an amino acid sequence of S/T I/V H X, I S/T I/V H X, or C/A I S/T I/V H X, and/or on the carboxy terminus side by an amino acid sequence of X E V/I R, X E V/I R Y/F, or X E V/I R Y/F L, wherein “X” indicates the aspartic acid amino acid that is mutated or will be mutated to alanine.

As used herein, the terms “depleted,” “depletion,” or “depleter” with respect to mtDNA refers to a decrease in mtDNA copy number and/or concentration in a human or in a non-human animal, tissue, or cell of the non-human animal. Such a determination may be made with regard to a human that has not been administered a compound or composition of the disclosure or to a control non-human animal tissue, or cell (for example, a non-human animal that has not expressed or does not express a mutant POLG1 polypeptide).

As used herein, “treatment” of a disease or condition refers to reducing or lessening the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein, the term “effective amount” refers to an amount of a compound or composition of the disclosure administered to a subject, which is effective to provide a desired response and/or effect in the subject. The response and/or effect may be a cosmetic response and/or effect. The response and/or effect may be a therapeutic response and/or effect. The response and/or effect may be a prophylactic or preventative response and/or effect.

As used herein, the term “therapeutically effective amount” refers to an amount of compound or composition of the disclosure administered to a subject, which is effective to treat a disease or condition described herein in a subject and/or to produce a desired physiological response and/or therapeutic effect in the subject. One example of a desired physiological response includes increasing mtDNA copy number and/or concentration.

The actual dose which comprises the “effective amount” or “therapeutically effective amount” may depend upon the route of administration, the size and health of the subject, the disorder being treated, and the like.

In some embodiments, the “effective amount” or “therapeutically effective amount” in the context of the present disclosure is sufficient to induce mitochondrial biogenesis. The “effective amount” or “therapeutically effective amount” may be sufficient to induce mitochondrial biogenesis locally. The “effective amount” or “therapeutically effective amount” may be sufficient to induce mitochondrial biogenesis systemically.

In some embodiments, the “effective amount” or “therapeutically effective amount” in the context of the disclosure decreases an inflammatory phenotype, increases expression of mitochondrial oxidative phosphorylation complexes, increases collagen content of the skin, decreases epidermal thickness, decreases epidermal hyperplasia, decreases acanthosis, decreases hyperkeratosis, decreases wrinkle length, decreases wrinkle depth, decreases the number of wrinkles in a defined area, increases spacing between wrinkles, decreases hyperpigmentation, decreases hypopigmentation, increases stability of mitochondrial oxidative phosphorylation complexes, alters, e.g., decreases expression of at least one gene selected from the group consisting of: NF-κB, COX-2, INF-β1, CCL5, MMP1, MMP2, MMP9, MMP13, IGF1R, VEGF, and MRPS5, alters, e.g., increases expression of at least one gene selected from the group consisting of: TIMP1 KLOTHO, COL1A1, MTCO2, TFAM, and VDAC, activates a gene associated with mitochondrial health and activity, selected from: FGF2, FGFR1, COX7A1, PDK4, FAM173A, MRPL12, and WNT11, decreases inflammatory infiltrate in the skin and/or hair follicles, decreases aberrant hair follicles, such as, but not limited to, aberrant telogen and anagen hair follicles with defective sebaceous glands), decreases hair loss, decreases hair thinning, increases an amount of hair follicles in growth phase, decreases hair graying, decreases incidence of hair with an irregular hair shaft, decreases incidence of hair with a rough outer surface, decreases incidence of hair with a medulla of irregular width, decreases incidence of hair with an irregular septation, and/or decreases incidence of hair with a medullary space having decreased melanin concentration in a subject, and/or increases expression of at least one protein selected from PGC-1a, TFAM, NRF-1, and COXII.

In some embodiments, the “effective amount” or “therapeutically effective amount” in the context of the disclosure increases mtDNA copy number or concentration by at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In each of the foregoing, when a reduction or increase is specified, such reduction or increase may be determined with respect to a subject or non-human animal that has not been treated with a compound or composition of the disclosure and that is suffering from a disease or condition described herein.

In some embodiments, the “effective amount” or “therapeutically effective amount” in the context of the disclosure prevents incidence of an inflammatory phenotype, prevents a decrease in expression of mitochondrial oxidative phosphorylation complexes, prevents a decrease in stability of mitochondrial oxidative phosphorylation complexes, prevents a decrease in collagen content of the skin, prevents an increase in epidermal thickness, prevents an increase in epidermal hyperplasia, prevents an increase in acanthosis, prevents an increase in hyperkeratosis, prevents an increase in wrinkle length, prevents an increase in wrinkle depth, prevents an increase in the number of wrinkles in a defined area, prevents an increase in spacing between wrinkles, prevents an increase in hyperpigmentation, prevents an increase in hypopigmentation, prevents an increase in expression of at least one gene selected from the group consisting of: NF-κB, COX-2, INF-β1, CCL5, MMP1, MMP2, MMP9, MMP13, IGF1R, VEGF, and MRPS5, prevents a decrease in expression of at least one gene selected from the group consisting of: TIMP1, KLOTHO, COL1A1, MTCO2, TFAM, and VDAC, prevents a deactivation of a gene associated with mitochondrial health and activity, selected from: FGF2, FGFR1, COX7A1, PDK4, FAM173A, MRPL12, and WNT11, prevents an increase in inflammatory infiltrate in the skin and/or hair follicles, prevents an increase in aberrant hair follicles, such as, but not limited to, aberrant telogen and anagen hair follicles with defective sebaceous glands), prevents an increase in hair loss, prevents an increase in hair thinning, prevents a decrease in amount of hair follicles in growth phase, prevents an increase in hair graying, prevents an increase in incidence of hair with an irregular hair shaft, prevents an increase in incidence of hair with a rough outer surface, prevents an increase in incidence of hair with a medulla of irregular width, prevents an increase in incidence of hair with an irregular septation, prevents an increase in incidence of hair with a medullary space having decreased melanin concentration in a subject, prevents a decrease in mtDNA copy number or concentration, and/or prevents a decrease in expression of at least one protein selected from PGC-1a, TFAM, NRF-1, and COXII.

As used herein, the term “excipient” means a substance formulated alongside the active ingredient of a composition included for purposes such as, but not limited to, long-term stabilization, bulking up solid formulations that contain potent active ingredients in small amounts (thus often referred to as bulking agents, fillers, or diluents), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. The excipient may be a pharmaceutically acceptable excipient. The excipient may be a cosmetically acceptable excipient. Examples of suitable excipients are described in “Remington's Pharmaceutical Sciences” by E. W. Martin.

As used herein, the term “in need of” (such as in the phrase “in need of treatment”) refers to a judgment made by a healthcare professional that a subject requires or will benefit from administration of a compound of the disclosure. This judgment is made based on a variety of factors that are in the realm of a healthcare professional's expertise, such as, but not limited to, the knowledge that the subject is ill, or will be ill, as the result of a disease or condition that is treatable by a method or drug composition of the disclosure.

As used herein, the terms “mutant POLG1” or “mutated POLG1” refers to a POLG1 amino acid sequence from a particular species that contains at least one mutation as compared to the wild-type POLG1 sequence from that species. A mutation need not cause a disease. A single mutation or more than one mutation may be present. In a particular embodiment, a single dominant negative mutation may be present (such as, but not limited to, a D1135A mutation), optionally with one or additional mutations.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “cosmetically acceptable” refers to a compound that is compatible with the other cosmetic ingredients of a composition and not deleterious to the subject receiving the compound or composition. A cosmetically acceptable composition or compound may be pharmaceutically acceptable. However, a cosmetically acceptable composition or compound need not be pharmaceutically acceptable.

As used herein, the term “pharmaceutically acceptable form” is meant to include known forms of a compound of the disclosure that may be administered to a subject, including, but not limited to, solvates, hydrates, prodrugs, isomorphs, polymorphs, pseudomorphs, neutral forms and salt forms of a compound of the disclosure.

As used herein, the term “pharmaceutically acceptable salt” refers to physiologically and pharmaceutically acceptable salts of the compounds of the invention: i.e., salts that retain the desired biological activity of the parent compound and do not impart undesired toxicological effects to the compounds disclosed. For oligonucleotides, exemplary pharmaceutically acceptable salts include but are not limited to (a) salts formed with cations such as sodium, potassium, ammonium, magnesium, calcium, polyamines such as spermine and spermidine and the like; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

As used herein, the term “pharmaceutical composition” refers to a mixture of one or more of the compounds of the disclosure, with other components, such as, but not limited to, pharmaceutically acceptable carriers and/or excipients. The purpose of a pharmaceutical composition is to facilitate administration of a compound of disclosure.

In some embodiments, a “cosmetically acceptable” excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

As used herein, the terms “repleted,” “repletion,” or “repleter” with respect to mtDNA refers to an increase in mtDNA copy number and/or concentration in a human or a non-human animal, tissue, or cell. Such a determination may be made with regard to a human or to a control non-human animal tissue, or cell that has undergone mtDNA depletion (such as immediately before repletion is initiated. In certain aspects, mtDNA is repletion results in mtDNA copy number and/or concentration approximately equal to the mtDNA copy number and/or concentration observed in a control non-human animal, tissue, or cell (for example, a non-human animal that has not expressed or does not express a mutant POLG1 polypeptide).

As used herein, the term “solvate” means a compound of the disclosure, or a pharmaceutically acceptable salt thereof, wherein one or more molecules of a suitable solvent are incorporated in the crystal lattice. A suitable solvent is physiologically tolerable at the dosage administered. Examples of suitable solvents are ethanol, water and the like. When water is the solvent, the molecule may be referred to as a “hydrate.”

As used herein, the terms “subject” or “patient” include all members of the animal kingdom including, but not limited to, vertebrates, mammals, animals (e.g., cats, dogs, horses, swine, rodents, etc.) and humans. In certain embodiments, the subject is a human. In certain embodiments, the subject is an animal, e.g., a research animal, e.g., a mouse, a rat.

As used herein, the terms “similarity” or “similarity score” when used with respect to a compound refers to a degree of similarity between two or more compounds in chemical structure, chemical function, and/or one or more chemical properties. The similarity score may be quantified by augmenting data generated by a deep neural network trained to model a plurality of chemical reactions for any given compound. The data may be augmented by a multi-dimensional vector defining a matrix of properties of the compound or a chemical reaction involving the compound to generate an embedding score for the compound. The embedding score for two or more compounds may be compared to generate the similarity score between the two or more compounds.

All patent applications, patents, and printed publications cited herein are incorporated herein by reference in their entireties, for all purposes.

### Impact of Mitochondrial Function on Intrinsic and Extrinsic Aging

Mitochondrial dysfunction is implicated in both intrinsic and extrinsic aging. The presence of skin wrinkles, acanthosis, epidermal hyperplasia with hyperkeratosis, and marked inflammatory infiltrate in the skin has been observed in mtDNA-depleter mice and represent characteristics similar to the extrinsic aging of skin in humans. Furthermore, the changes in expression of intrinsic aging-associated genetic markers support intrinsic mechanisms underlying the phenotypic changes observed in mtDNA-depleter mice.

Loss of collagen fibers is reported to underlie skin wrinkles. A tight balance between the proteolytic matrix metalloprotease (MMP) enzymes and their tissue-specific inhibitor tissue inhibitor metalloproteinase-1 (TIMP1) is essential to maintain the collagen fiber content in the skin. Expression of MMPs is altered in the aged skin. Consistent with these reports, the skin of mtDNA-depleter mice showed increased expression of MMPs and decreased expression of TIMP1, indicating loss of balance contributing to the development of skin wrinkles. Repletion of mtDNA content restored MMP expression leading to a reversal of wrinkled skin and hair loss. These experiments show that mitochondria are regulators of aging. This observation is surprising and suggests that epigenetic mechanisms underlying mitochondria-to-nucleus cross-talk must play an important role in the restoration of normal skin and hair phenotype.

mtDNA stress triggers inflammatory response Inflammation also underlies aging and age-related diseases. Increased levels of markers of inflammation in the mtDNA-depleter mice indicate an activated immune response in the skin of mtDNA-depleter mice. Increased expression of NF-κB, a master regulator of the inflammatory response upon mtDNA depletion and its reduced expression after the restoration of mtDNA content suggests that NF-κB signaling is a critical mechanism contributing to the skin and hair follicle pathologies observed in mtDNA-depleter mice. Furthermore, a unique feature of proteins encoded by mtDNA is N-formyl-methionine at the N terminus. N-formylated peptides when present in the extracellular space are known to act as mitochondrial damage-associated molecular patterns and activate neutrophils or activate keratinocyte-intrinsic responses resulting in the recruitment of immune cells. While previous animal models have shown alternations in mtDNA homeostasis and/or mtDNA copy number or concentration using a localized approach (for example targeting only a specific cell type), the disclosure utilizes an animal model that provides for the global and targeted disruption of mtDNA homeostasis and/or mtDNA copy number of concentration in a controlled manner. Using such an animal model, the disclosure identified compounds that are effective in treating diseases and conditions relating to mitochondrial dysfunction in a background of significant mtDNA depletion. In addition, the disclosure demonstrates that such compounds reverse the physiological and phenotypic effects mediated by mitochondrial dysfunction. Exemplary physiological and phenotypic effects of intrinsic and extrinsic aging reversed include a decrease in skin wrinkles, a decrease in hair loss, an increase in hair follicles in growth phase, decreased inflammatory gene expression, decreased inflammatory infiltrate in the skin and hair follicles, and increased collagen content in the skin.

Further, due to the short lifespan of the animal models of the conventional practice, prior studies were prevented from determining the effect of mitochondrial dysfunction on the aging process and such studies did not observe the appearance of many of the physiological and phenotypic changes related to mitochondrial dysfunction. As such, the conventional practice was not capable of identifying compounds effective to treat such physiological and phenotypic changes related to mitochondrial dysfunction reported herein.

The foregoing limitations make identifying effective treatments for the skin diseases and conditions where mitochondrial dysfunction is at issue difficult. In addition, due to aberrations in normal epithelial development and hair follicle morphogenesis in current models, the issue of false positive and false negative results is high. The disclosure utilized an inducible non-human animal model expressing a mutated POLG1 polypeptide (such as, but not limited to, a POLG1 polypeptide expressing a dominant-negative (DN) mutation) that induces mitochondrial dysfunction (for example by depletion of mtDNA) in the whole animal or selected cells/tissues. The non-human animal model allows for the ubiquitous suppression and restoration of mitochondrial function in the whole animal or in specific cells/tissues.

The non-human animal model disclosed can be used to rapidly identify compounds effective in treating mtDNA-related diseases and conditions. The animal model in the absence of the expression of mutated POLG1 expression maintained normal epidermal differentiation and hair follicle morphogenesis making this animal model system a valuable tool in identifying therapies for the treatment of a variety of diseases and conditions characterized by mitochondrial dysfunction. When the mutant POLG1 polypeptide is expressed, the animal model demonstrates profound phenotypic age-related changes in the skin, including development of skin wrinkles and hair loss. The phenotypic changes are reversible when mitochondrial function is restored (such as through the administration of an extract or compound described herein).

### Non-Human Animal Model

In certain aspects, the methods described herein utilize a genetically modified non-human animal that expresses a mutant POLG1 polypeptide in a controlled manner. Tissues, organs and cells from such animal model may also be used. In one embodiment, the mutant POLG1 polypeptide is expressed ubiquitously (in every cell of the non-human animal). In another embodiment, the mutant POLG1 polypeptide is expressed in a specific tissue or set of tissues or in a specific cell type. Such non-human animal model is described in US Patent Publication No. 2020-0085021-A1, incorporated herein by reference in its entirety for all purposes.

In a particular aspect, the animal model exhibits at least one characteristic selected from the group consisting of: reduced mitochondrial (mt) DNA content, reduced mtDNA copy number, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, altered expression of at least one gene selected from the group consisting of: NF-κB, COX-2, INF-β1, CCL5, MMP1, MMP2, MMP9, MMP13, IGF1R, VEGF, and MRPS5, altered expression of at least one gene selected from the group consisting of: TIMP1 and KLOTHO, increased skin inflammation, and aberrant hair follicles.

### Methods of Screening for Therapeutic Agents

The disclosure provides an artificial neural network trained to model a plurality of chemical reactions for any given compound. Through the use of the artificial neural network described herein, compounds and compositions having a similarity score with a known promoter or inhibitor of mitochondrial biogenesis may be reliably identified. The similarity score may be quantified by augmenting data generated by the deep neural network in a multi-dimensional vector defining a matrix of properties of the compound or a chemical reaction involving the compound to generate an embedding score for the compound. The embedding score for the compound may be compared to an embedding score for the known promotor or inhibitor of mitochondrial biogenesis to generate the similarity score. Therefore, the disclosure provides for methods of screening compounds and compositions having a sufficient similarity score with known compounds. The identified compounds are predicted to be effective at promoting or inhibiting mitochondrial biogenesis.

The disclosure provides a non-human animal model comprising a mutant POLG1 polypeptide and that express the mutant POLG1 polypeptide in a controlled manner either throughout the animal or in specific tissues. Through the use of the non-human animal model described herein, compounds and compositions effective in treating diseases and conditions related to mitochondrial dysfunction can be reliably identified. Methods utilizing cells, and tissues from such a non-human animal are also provided. Therefore, the disclosure provides for methods of screening compounds and compositions effective to treat diseases and conditions related to mitochondrial dysfunction in a subject, including diseases and conditions related to mtDNA depletion.

In one embodiment, the disclosure provides for identification of a compound for the treatment of a disease or condition due, at least in part, to mitochodrial dysfunction, including changes in mtDNA copy number and/or concentration, and/or dysfunctional mitochondrial OXPHOS. Such diseases and conditions include, but are not limited to, mtDNA depletion syndromes, mitochondrial diseases, aging, aging-associated chronic diseases or conditions, reduced energy levels and vitality, characteristics of hair aging including hair loss and graying, characteristics of skin aging including skin wrinkles and senile lentigines, and other human pathologies. Exemplary mitochondrial diseases include cardiovascular disease, diabetes, cancer, neurological disorders, such as age-associated neurological disorders, skin diseases and conditions, e.g., skin wrinkles, changes in skin pigmentation, senile lentigines, characteristics of skin aging, hair or scalp diseases and conditions, e.g., hair loss, hair thinning, changes in hair pigmentation, e.g., hair graying.

In one embodiment, such a method of screening comprises the steps of: a) providing a deep neural network trained to model a plurality of chemical reactions for a known promoter or inhibitor of mitochondrial biogenesis; b) executing the deep neural network to identify one or more compounds having a threshold similarity score of at least 70% with the known promoter or inhibitor of mitochondrial biogenesis; c) selecting one or more identified compounds on the basis of at least one inclusion criteria; and d) evaluating the at least one selected compound in an assay to determine an effect on mitochondrial biogenesis by the selected compound, wherein the assay may include an in vitro assay, ex vivo assay, or in vivo assay. The at least one inclusion criteria may include a structural, functional, physical, or chemical property of the identified compound. Exemplary properties include size, charge, ionic strength, bond strength, valence, hybridization, micromolecular structure, macromolecular structure, and others. The threshold similarity score may be at least 70%, at least 75%, at least 80%, at least 85%, at least 90%, at least 95%, at least 99%, at least 99.5%, at least 99.9%, at least 99.95%, at least 99.99%, at least 99.995%, at least 99.999%, or greater.

In some embodiments, the assay may include screening the at least one selected compound with a non-human animal capable of inducible expression of a mutant POLG1 polypeptide, as described herein. In some embodiments, the assay may include screening the at least one selected compound to measure an effect on protein or gene expression of a mitochondrial health biomarker, as identified herein. The method of screening may be repeated by executing the deep neural network to identify one or more compounds having a similarity score of at least 70% with at least one identified compound and/or at least one selected compound. In some embodiments, the deep neural network may be retrained with at least one identified compound and/or at least one selected compound.

In one embodiment, such a method of screening comprises the steps of: a) providing a non-human animal capable of inducible expression of a mutant POLG1 polypeptide; b) stimulating the expression of the mutant POLG1 polypeptide, wherein stimulating expression of the mutant POLG1 polypeptide induces a physiological or phenotypic response; c) administering an agent to the non-human animal either before step b) or after step b); d) determining the effect of the agent on pathology; and e) comparing the effect of the agent to a control animal, wherein a reduction or an increase (as appropriate) in the physiological or phenotypic response in the non-human animal after administration of the agent indicates the agent is a therapeutic agent for the treatment of the physiological or phenotypic response.

In another embodiment, the disclosure provides a method for identifying a therapeutic agent for the treatment of mitochondrial dysfunction.

In another embodiment, the disclosure provides a method for identifying a therapeutic agent for the treatment of a disease or condition associated with mitochondrial dysfunction or a symptom thereof.

In another embodiment, the disclosure provides a method for identifying a therapeutic agent for the treatment of an aging-associated chronic disease or condition related to mitochondrial dysfunction or a symptom thereof.

In another embodiment, the disclosure provides a method for identifying a therapeutic agent for improving or preventing a decrease in energy level and/or vitality.

In another embodiment, the disclosure provides a method for identifying a therapeutic agent for treatment of a characteristic of skin aging. A characteristic of skin aging includes, but is not limited to, skin wrinkles, increased wrinkle length, increased wrinkle depth, increased in the number of wrinkles in a defined area, decreased spacing between wrinkles, hair loss, hair thinning, hair graying, decrease in amount of hair follicles in the growth phase, hair with an irregular hair shaft, hair with a rough outer surface, hair with a medulla of irregular width, hair with an irregular septation, hair with a medullary space having decreased melanin concentration, or a combination of the foregoing.

In another embodiments, the disclosure provides a method for identifying a therapeutic agent for treatment of a skin disease or condition. The skin condition (including skin wrinkles and/or wrinkled skin and/or hair loss and/or hair thinning) may be characterized by one or more of the following: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, epidermal hyperplasia, acanthosis, hyperkeratosis, altered expression of at least one gene selected from the group consisting of: NF-κB, COX-2, INFβ-1, CCL5, MMP1, MMP2, MMP9, MMP13, IGF1R, VEGF, and MRPS5, altered expression of at least one gene selected from the group consisting of: TIMP1, KLOTHO, COL1A1, MTCO2, TFAM, and VDAC, increased inflammatory infiltrate in the skin and/or hair follicles, aberrant hair follicles, such as, but not limited to, aberrant telogen and anagen hair follicles with defective sebaceous glands), or a combination of the foregoing.

The skin condition may be characterized as an appearance or effect of aging. The skin condition may be characterized as a condition of skin surface topology. The skin condition may be characterized as a condition of skin integrity. Thus, a reduction in the skin condition or a characteristic of the skin condition may be identified by a modulation of an appearance or effect of aging, a modulation of skin surface topology, and/or a modulation of skin integrity.

In another embodiments, the disclosure provides a method for identifying a therapeutic agent for treatment of skin wrinkles. Skin wrinkles may be associated with one or more of the following: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, epidermal hyperplasia, acanthosis, hyperkeratosis, altered expression of at least one gene selected from the group consisting of: NF-κB, COX-2, INFβ-1, CCL5, MMP1, MMP2, MMP9, MMP13, IGF1R, VEGF, and MRPS5, altered expression of at least one gene selected from the group consisting of: TIMP1, KLOTHO, COL1A1, MTCO2, TFAM, and VDAC, increased inflammatory infiltrate in the skin and/or hair follicles, aberrant hair follicles, such as, but not limited to, aberrant telogen and anagen hair follicles with defective sebaceous glands), or a combination of the foregoing.

The parameter of a skin wrinkle may be wrinkle length, wrinkle depth, the number of wrinkles in a defined area, and/or the spacing between wrinkles. The number of wrinkles in a defined area can be determined empirically. In one embodiment, the method for measuring a parameter of skin wrinkles is described in International Application Publication No. WO 2013/112974A1 filed Jan. 27, 2013, incorporated herein by reference in its entirety for all purposes. Such a method comprises determining the length of a wrinkle and at least one other measured physical characteristic of a skin wrinkle, such as wrinkle depth, the number of wrinkles in a defined area, and/or the spacing between wrinkles. Further, the skin wrinkles parameters may be used to define a severity level of the skin wrinkle, the severity level being based on a combination of wrinkle length and the at least on other measured physical characteristic of a skin wrinkle. The number of wrinkles can be determined empirically. For example, the number of skin wrinkles in a defined area may be used.

In another embodiments, the disclosure provides a method for identifying a therapeutic agent for treatment of hair loss or hair thinning.

Hair loss and hair thinning may be associated with one or more of the following: altered expression of at least one gene selected from the group consisting of: NF-κB, COX-2, INF-β1, CCL5, MMP1, MMP2, MMP9, MMP13, IGF1R, VEGF, and MRPS5, altered expression of at least one gene selected from the group consisting of: TIMP1, KLOTHO, COL1A1, MTCO2, TFAM, and VDAC, increased inflammatory infiltrate in the hair follicles, and an increase in aberrant hair follicles, such as, but not limited to, aberrant telogen and anagen hair follicles with defective sebaceous glands, or a combination of the foregoing.

In certain embodiments, hair loss and hair thinning are quantified visually through the use of photographs. The photographs may be taken with the aid of a stereotactic positioning device on which camera is mounted, to assure that the view, magnification and lighting are consistent over different measurement periods.

In another embodiments, the disclosure provides a method for identifying a therapeutic agent for treatment of senile lentigines. Senile lentigines may be associated with one or more of the following: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, epidermal hyperplasia, acanthosis, hyperkeratosis, altered expression of at least one gene selected from the group consisting of: NF-κB, COX-2, INF-β1, CCL5, MMP1, MMP2, MMP9, MMP13, IGF1R, VEGF, and MRPS5, altered expression of at least one gene selected from the group consisting of: TIMP1, KLOTHO, COL1A1, MTCO2, TFAM, and VDAC, increased inflammatory infiltrate in the skin and/or hair follicles, aberrant hair follicles, such as, but not limited to, aberrant telogen and anagen hair follicles with defective sebaceous glands, or a combination of the foregoing.

In another embodiments, the disclosure provides a method for identifying a therapeutic agent for preventing or treating a change in skin or hair pigmentation. Changes in skin or hair pigmentation may be associated with one or more of the following: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, epidermal hyperplasia, acanthosis, hyperkeratosis, altered expression of at least one gene selected from the group consisting of: NF-κB, COX-2, INFβ-1, CCL5, MMP1, MMP2, MMP9, MMP13, IGF1R, VEGF, and MRPS5, altered expression of at least one gene selected from the group consisting of: TIMP1, KLOTHO, COL1A1, MTCO2, TFAM, and VDAC, increased inflammatory infiltrate in the skin and/or hair follicles, aberrant hair follicles, such as, but not limited to, aberrant telogen and anagen hair follicles with defective sebaceous glands, or a combination of the foregoing.

In any of the described methods of screening, agents can include, but are not limited to, chemical compounds, pharmaceutical compositions, cosmetic composition, extracts, plant extracts, seaweed extracts, microbial extracts, biological compounds and compositions (e.g., proteins, DNA, RNA, siRNAs, vaccines and the like), and microorganisms. Further, the agent may be selected from a library, including a library of agents approved by a regulatory authority such as the FDA.

In any of the described methods of screening, any of the transgenic non-human animals of the disclosure may be used.

In any of the described methods of screening, step b) may be accomplished by providing an inducer compound to the transgenic non-human animal or withholding the inducer compound from the transgenic non-human animal.

In any of the described methods of screening, the agent is added before step b). In any of the described methods of screening, the agent is added after step b).

In any of the described methods of screening, the animal model is an animal model described in the preceding section. In any of the described methods of screening, the mutant POLG1 polypeptide may be any mutant POLG1 polypeptide described herein. In certain aspects, the mutant POLG1 polypeptide comprises a dominant negative mutation. In certain aspects, the mutant POLG1 polypeptide comprises a D1135A mutation.

### Methods of Treatment

The disclosure provides compounds and compositions effective to treat or prevent diseases and conditions related to mitochondrial dysfunction, including mtDNA depletion. In certain embodiments, the diseases and conditions related to mitochondrial dysfunction, including mtDNA depletion, include treating or preventing an aging-associated chronic disease or condition, improving or preventing a decrease in energy levels and vitality treating or preventing a characteristic of aging, and/or treating, improving, or preventing skin diseases or conditions. While not being bound to any particular theory, such compounds may exert the observed effects through inhibiting a decrease in mitochondrial DNA copy number or concentration, inhibiting depletion of mitochondrial DNA, inhibiting degradation of mitochondrial DNA, contributing to an increase in mitochondrial DNA copy number or concentration, repleting mitochondrial DNA, and/or promoting increased activity of mitochondrial DNA.

In one embodiment, the disclosure provides a method for treating or preventing mitochondrial dysfunction in a subject, the method comprising administering to said subject an effective amount of a compound of the disclosure, or a pharmaceutically acceptable form thereof.

In one embodiment, the disclosure provides a method for treating or preventing a disease or condition associated with mitochondrial dysfunction or a symptom thereof in a subject, the method comprising administering to said subject an effective amount of a compound of the disclosure, or a pharmaceutically acceptable form thereof.

In one embodiment, the disclosure provides a method for treating or preventing an aging-associated chronic disease or condition related to mitochondrial dysfunction or a symptom thereof in a subject, the method comprising administering to said subject an effective amount of a compound of the disclosure, or a pharmaceutically acceptable form thereof.

In one embodiment, the disclosure provides a method for treating or preventing a characteristic of aging related to mitochondrial dysfunction or a symptom thereof in a subject, the method comprising administering to said subject an effective amount of a compound of the disclosure, or a pharmaceutically acceptable form thereof.

In one embodiment, the disclosure provides a method for improving or preventing a decrease in energy level and/or vitality in a subject, the method comprising administering to said subject an effective amount of a compound of the disclosure, or a pharmaceutically acceptable form thereof.

In one embodiment, the disclosure provides a method for improving or preventing a skin disease or condition in a subject, the method comprising administering to said subject an effective amount of a compound of the disclosure, or a pharmaceutically acceptable form thereof.

Exemplary diseases or conditions associated with mitochondrial dysfunction include cardiovascular disease, diabetes, cancer, and neurological disorders, such as aging-associated neurological disorders. Thus, exemplary aging-associated diseases related to mitochondrial dysfunction include neurological disorders.

Examples of treating or preventing a characteristic of aging includes, but is not limited to, treating and/or preventing skin wrinkles, decreasing wrinkle length, decreasing wrinkle depth, decreasing the number of wrinkles in a defined area, increasing the spacing between wrinkles, preventing hair loss, preventing hair graying, providing hair with a regular hair shaft, providing hair with a smooth outer surface, providing hair with a medulla of regular width, providing hair with a regular septation, providing hair with a medullary space filled with melanin, or a combination of the foregoing. In certain aspects, treating or preventing a characteristic of aging includes treating and/or preventing skin wrinkles. In certain aspects, treating or preventing a characteristic of aging includes preventing hair loss. In certain aspects, treating or preventing a characteristic of aging includes preventing hair graying. In certain aspects, treating or preventing a characteristic of aging includes preventing a change in skin or hair pigmentation. In certain aspects, treating or preventing a characteristic of aging includes treating or preventing senile lentigines.

In some embodiments, the characteristic of aging may be triggered by exposure to a chemical or therapeutic agent. For instance, the characteristic of aging may be a side effect of a chemical or therapeutic agent. In certain embodiments, the characteristic of aging may be a side effect of chemotherapy and/or radiation therapy. Exemplary side effects of chemotherapy and/or radiation therapy that may be treated, limited, inhibited, or prevented by the methods disclosed herein include hair loss, skin wrinkles, changes in skin elasticity, changes in skin or hair pigmentation, blistering, scarring, or photoaging. In certain embodiments, the characteristic of aging may be triggered by an environmental factor, e.g., exposure to ultraviolet radiation, extreme cold, or extreme heat.

In accordance with one or more aspects, a method of reducing an appearance or effect of aging in a subject is disclosed. The method may comprise administering to said subject an effective amount of a compound of the disclosure, or a pharmaceutically acceptable form thereof, thereby reducing the appearance or effect of aging in the subject.

In some embodiments, skin diseases or conditions include aging-associated diseases or conditions, as previously described. In some embodiments, the skin diseases or conditions are not associated with age. The skin diseases or conditions may be associated with a course of treatment, e.g., chemotherapy or radiation therapy. The skin diseases or conditions may be genetic. The skin diseases or conditions may be associated with an autoimmune disease. The skin diseases or conditions may be associated with environmental factors, such as exposure to ultraviolet radiation, extreme cold, or extreme heat. Thus, the skin diseases or conditions treatable by the methods disclosed herein may be aging-associated or not.

Exemplary skin diseases or conditions include skin wrinkles, hair loss, hair thinning, changes in skin or hair pigmentation, e.g., hair graying, senile lentigines, diseases or conditions of the sebaceous glands, psoriasis, skin rashes, acrocyanosis, and others.

In accordance with one or more aspects, a method of treating skin of a subject is disclosed. The method may comprise administering to said subject an effective amount of a compound of the disclosure, or a pharmaceutically acceptable form thereof, thereby treating the skin of the subject.

In accordance with one or more aspects, a method of improving skin integrity in a subject is disclosed. The method may comprise administering to said subject an effective amount of a compound of the disclosure, or a pharmaceutically acceptable form thereof, thereby improving the skin integrity of the subject.

In accordance with one or more aspects, a method of improving skin surface topology in a subject is disclosed. The method may comprise administering to said subject an effective amount of a compound of the disclosure, or a pharmaceutically acceptable form thereof, thereby improving the skin surface topology of the subject.

In accordance with one or more aspects, a method for reducing or preventing degradation of collagen in the skin of a subject or increasing the collagen content in the skin of a subject is disclosed. The method may comprise administering to said subject an effective amount of a compound of the disclosure, or a pharmaceutically acceptable form thereof, thereby reducing or preventing degradation of collagen in the skin of a subject or increasing the collagen content in the skin of the subject.

In accordance with one or more aspects, a method for reducing or preventing inflammation in the skin or a hair follicle of a subject is disclosed. The method may comprise administering to said subject an effective amount of a compound of the disclosure, or a pharmaceutically acceptable form thereof, thereby reducing or preventing inflammation in the skin or a hair follicle of the subject.

In accordance with one or more aspects, a method of preventing, limiting, or inhibiting progression of an appearance or effect of aging in the skin of a subject is disclosed. The method may comprise administering to said subject an effective amount of a compound of the disclosure, or a pharmaceutically acceptable form thereof, thereby preventing, limiting, or inhibiting progression of the appearance or effect of aging in the skin of the subject.

In accordance with one or more aspects, a method of preventing, limiting, or inhibiting skin inflammation in a subject is disclosed. The method may comprise administering to said subject an effective amount of a compound of the disclosure, or a pharmaceutically acceptable form thereof, thereby preventing, limiting, or inhibiting skin inflammation of the subject.

In accordance with one or more aspects, a method of preventing, limiting, or inhibiting injury to skin integrity or condition in a subject is disclosed. The method may comprise administering to said subject an effective amount of a compound of the disclosure, or a pharmaceutically acceptable form thereof, thereby preventing, limiting, or inhibiting injury to skin integrity or condition of the subject.

In accordance with one or more aspects, a method of substantially maintaining a state of skin integrity or condition in a subject is disclosed. The method may comprise administering to said subject an effective amount of a compound of the disclosure, or a pharmaceutically acceptable form thereof, thereby substantially maintaining the state of skin integrity or condition of the subject.

In accordance with one or more aspects, a method of preventing, limiting, or inhibiting a topographical change of a cutaneous layer of skin of a subject is disclosed. The method may comprise administering to said subject an effective amount of a compound of the disclosure, or a pharmaceutically acceptable form thereof, thereby preventing, limiting, or inhibiting the topographical change of the cutaneous layer of skin of the subject.

In accordance with one or more aspects, a method of treating or preventing a change in skin or hair pigmentation of a subject is disclosed. The method may comprise administering to said subject an effective amount of a compound of the disclosure, or a pharmaceutically acceptable form thereof, thereby treating or preventing a change in the skin or hair pigmentation of the subject.

Treating or preventing a change in skin or hair pigmentation may include treating, preventing, limiting, or inhibiting a change in melanocyte function. The change in melanocyte function may be associated with one or more of vitiligo, senile lentigines, mottled pigmentation, reticular pigmentation, punctate pigmentation, ectopic pigmentation, perifollicular pigmentation, interfollicular pigmentation, hypopigmentation or hyperpigmentation (e.g., patchy, focal, or diffuse hypopigmentation or hyperpigmentation), erythema, scaling, and telangiectasia. The change in skin or hair pigmentation and/or melanocyte function may be local or systemic.

Treating or preventing a change in skin or hair pigmentation may include treating, preventing, limiting, or inhibiting a change in dermal pigmentation and/or epidermal pigmentation (e.g., basal and/or suprabasal pigmentation). In certain embodiments, the change may comprise formation of localized high and low pigmentation in a dermal layer. In some embodiments, the change may comprise increased pigmentation in an epidermal layer. The change in skin or hair pigmentation may be associated with hyperplasia, acanthosis, hyperkeratosis, parakeratosis, orthokeratosis, hypomelanosis, melanogenesis, and/or increase epidermal thickness.

In certain embodiments, the change in skin or hair pigmentation may be triggered by exposure to ultraviolet light. For instance, exposure to ultraviolet light may produce mutations in mtDNA, inducing mitochondrial dysfunction that contribute to photoaging.

In certain embodiments, the change in skin or hair pigmentation may be triggered by exposure to a chemical or therapeutic agent, e.g., chemotherapy or radiation therapy.

In certain embodiments, the change in skin or hair pigmentation may be triggered by aging.

In certain embodiments, the change in skin or hair pigmentation may comprise a change in pigment distribution. For instance, the change in skin or hair pigmentation may comprise a change in melanocyte morphology and/or color. The change in skin or hair pigmentation may comprise a change in epidermal melanocyte number. The change in skin or hair pigmentation may comprise a change in dermal melanocyte number. The change in skin or hair pigmentation may be associated with deregulated secretion of growth factors KGF, HGF, SCF, and/or EDN1.

In accordance with one or more aspects, a method of treating, preventing, limiting, or inhibiting senile lentigines is disclosed. The method may comprise reducing a number and/or size of senile lentigines. The method may comprise preventing incidence of senile lentigines. The method may comprise inhibiting growth of senile lentigines. The senile lentigines may be associated with hyperpigmentation. The senile lentigines may be associated with hypopigmentation. The senile lentigines may be associated with an increase in number of melanocytes in lesional over perilesional epidermis. The senile lentigines may be associated with an increase in melanocyte cell number, increase in dendricity, elongation of dendrites, increased granularity of dermal melanocytes, keratinocyte senescence, and/or increased epidermal thickness.

In another embodiment, the disclosure provides a method for treating skin wrinkles in a subject, the method comprising administering to said subject an effective amount of a compound of the disclosure, or a pharmaceutically acceptable form thereof. In certain aspects of this embodiment, the administration decreases wrinkle length, decreases wrinkle depth, decreases the number of wrinkles in a defined area, decreases the spacing between wrinkles, or a combination of the foregoing.

In another embodiment, the disclosure provides a method for treating hair loss in a subject, the method comprising administering to said subject an effective amount of a compound of the disclosure, or a pharmaceutically acceptable form thereof. In certain aspects of this embodiment, the administration prevents hair graying, provides hair with a regular hair shaft, provides hair with a smooth outer surface, provides hair with a medulla of regular width, provides hair with a regular septation, provides hair with a medullary space filled with melanin, or a combination of the foregoing.

The methods disclosed herein may comprise administering to said subject an effective amount of a promoter of mtDNA. The methods disclosed herein may comprise administering to said subject an effective amount of an inhibitor of mtDNA.

Certain extracts have been identified as comprising one or more compounds that promote and/or inhibit mtDNA. Emblica extract, fucus extract, and chebula extract are described herein. It should be understood that similar extracts, in particular, extracts comprising one or more compounds disclosed herein, compounds that are constituents of an extract disclosed herein, and/or compounds having a similarity score of at least 95% with one or more compounds disclosed herein, are expected to provide similar mtDNA promotion and/or inhibition. Accordingly, other extracts, compounds derived from, constituents of, or purified from other extracts, and compounds having a similarity score of at least 95% with compounds derived from, constituents of, or purified from other extracts are within the scope of the disclosure.

Exemplary extracts include Polygonum aviculare extract, Physalis gngulata extract, Dunaliella sauna extract, Camellia sinensis leaf extract, Tremella fuciformis sporocarp extract, Alteromonas ferment extract, Theobroma cacao (cocoa) seed extract, Vitis vinifera (grape) flower cell extract, Mirabilis Jalapa callus extract, Alteromonas ferment extract, and Vibrio alginolyticus ferment filtrate.

Accordingly, the compounds described herein may be derived from, purified from, or isolated from the extract. The compounds described herein may be derived from, purified from, or isolated from a source other than the extract. A compound constituent of an extract may be derived from, purified from, or isolated from another natural or artificial source. In other embodiments, the compounds described herein may be synthetic. For instance, the compounds of the disclosure may be synthesized in a laboratory, manufacturing, or other setting. The above applies to compounds contained in or constituents of an extract, as well as compounds having a high similarity score thereof.

The methods disclosed herein may comprise administering to said subject an effective amount of an emblica extract, or a pharmaceutically acceptable form thereof.

The methods disclosed herein may comprise administering to said subject an effective amount of a compound derived from, constituent of, or purified from an emblica extract, or a pharmaceutically acceptable form thereof, or a compound having a similarity score of at least 95% with the compound derived from, constituent of, or purified from an emblica extract, or a pharmaceutically acceptable form thereof.

The methods disclosed herein may comprise administering to said subject an effective amount of a fucus extract, or a pharmaceutically acceptable form thereof.

The methods disclosed herein may comprise administering to said subject an effective amount of a compound derived from, constituent of, or purified from a fucus extract, or a pharmaceutically acceptable form thereof, or a compound having a similarity score of at least 95% with the compound derived from, constituent of, or purified from a fucus extract, or a pharmaceutically acceptable form thereof.

The methods disclosed herein may comprise administering to said subject an effective amount of a chebula extract, or a pharmaceutically acceptable form thereof.

The methods disclosed herein may comprise administering to said subject an effective amount of a compound derived from, constituent of, or purified from a chebula extract, or a pharmaceutically acceptable form thereof, or a compound having a similarity score of at least 95% with the compound derived from, constituent of, or purified from a chebula extract, or a pharmaceutically acceptable form thereof.

The disclosure may generally be related to extracts and compounds derived from, constituents of, or purified from extracts. It should be understood that compounds having a similarity score of at least 95%, for example, at least 96%, at least 97%, at least 98%, at least 99%, at least 99.5%, at least 99.9%, at least 99.95%, at least 99.99%, at least 99.995%, or at least 99.999% with a compound derived from, constituents of, or purified from the extract may be utilized in any composition as described herein.

Without wishing to be bound by theory, it is believed the administration of the compositions disclosed herein may involve inhibition of a decrease in mitochondrial DNA copy number or concentration, inhibition of a depletion of mitochondrial DNA, inhibition of a degradation of mitochondrial DNA, or a combination of the foregoing. Without wishing to be bound by theory, it is believed the administration of the compositions disclosed herein may involve inducing mitochondrial biogenesis and/or improving mitochondrial function.

In accordance with certain aspects, administration of the compositions disclosed herein may involve an increase in expression of mitochondrial oxidative phosphorylation complexes, an increase in stability of mitochondrial oxidative phosphorylation complexes, and/or an increase in expression of at least one protein selected from PGC-1a, TFAM, NRF-1, and COXII.

In certain aspects, the emblica extract is derived from Emblica officinalis (also known as Phyllanthus emblica, Indian gooseberry, or by its Hindi name Amla). Such an extract from Emblica officinalis may be derived from any portion of the plant as desired. For example, the extract may be derived from the stem portion, the fruit portion, or both the stem portion and the fruit portion of Emblica officinalis. In preparing such an extract, the Emblica officinalis may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of an emblica extract or a compound derived from, constituent of, or purified from and emblica extract is from 1 to 100 mg such as 1 mg, 5, mg, 10 mg, 20 mg, 30 mg, 40 mg, 50 mg, 60 mg, 70 mg, 80 mg, 90 mg, or 100 mg.

In certain aspects, the compound derived from, constituent of, or purified from an emblica extract is a breakdown product of a tannin, wherein the emblica extract is optionally as described above. In certain aspects, the compound derived from, constituent of, or purified from an emblica extract is an ellagitannin. In certain aspects, the compound derived from, constituent of, or purified from an emblica extract is emblicanin A, emblicanin B, punigluconin, pedunculagin, and/or chebulinic acid.

In certain aspects, the compound is an ellagitannin or a compound having a similarity score of at least 95% with an ellagitannin. Exemplary ellagitannins and compounds having a high similarity score with such ellagitannins are listed in Table 1. Other compounds having a high similarity score with ellagitannins are within the scope of the disclosure.

TABLE 1
Ellagitannins and compounds having a high similarity score with ellagitannins

| Ellagitannin | Compound having high similarity score with ellagitannin |
|---|---|
| Emblicanin b | Eugeniin |
| Emblicanin a | 1,2,3,4-Tetrakis-O-Galloyl-Alpha-D-Glucose |
| Emblicanin a | 1,2,3,6-Tetragalloylglucose |
| Emblicanin b | Punicafolin |
| Emblicanin a | 4-hydroxy-3,5-bis(3,4,5-trihydroxybenzoyloxy)-6-[(3,4,5- |
| | trihydroxybenzoyloxy)methyl]oxan-2-yl 3,4,5-trihydroxybenzoate |
| Emblicanin b | Punicalin |
| Emblicanin a | Thonningianin A |
| Emblicanin a | Sagerinic Acid |
| Emblicanin b | Casuariin |
| Emblicanin a | Prunasin 2′,3′,4′,6′-Tetra-O-Gallate |
| Emblicanin b | Epigallocatechin-(4Beta->8)-Epigallocatechin-3-O-Gallate |
| Emblicanin b | Arjuna |
| Emblicanin a | Hyemaloside A |
| Emblicanin b | Trapain |
| Emblicanin a | 4-[3,4-dihydroxy-5-(3,4,5-trihydroxybenzoyloxy)benzoyloxy]-1- |
| | hydroxy-3,5-bis(3,4,5-trihydroxybenzoyloxy)cyclohexane-1- |
| | carboxylic acid |
| Emblicanin a | Pentagalloylglucose |
| Emblicanin a | Myricetin 3-(2″,3″-Digalloylrhamnoside) |
| Emblicanin b | Trilobatin G |
| Emblicanin b | Stachyurin |
| Emblicanin a | Eutannin |
| Emblicanin b | Thonningianin A |
| Emblicanin b | Castalagin |
| Emblicanin b | Casuarictin |
| Emblicanin a | Yunnaneic Acid G |
| Emblicanin a | Rabdosiin |
| Emblicanin b | 2-(3,4-dihydroxyphenyl)-5,7-dihydroxy-8-[3,5,7-trihydroxy-2- |
| | (3,4,5-trihydroxyphenyl)-3,4-dihydro-2H-1-benzopyran-4-yl]-3,4- |
| | dihydro-2H-1-benzopyran-3-yl 3,4,5-trihydroxybenzoate |
| Emblicanin b | Pterocaryanin C |
| Emblicanin b | Tercatain |
| Emblicanin a | Trapain |
| Emblicanin b | Tellimagrandin I |
| Emblicanin b | Sagerinic Acid |
| Emblicanin b | Paeonianin E |
| Emblicanin a | 2-(3-{[1-carboxy-2-(3,4-dihydroxyphenyl)ethoxy]carbonyl}-2- |
| | (3,4-dihydroxyphenyl)-7,8-dihydroxy-1,2-dihydronaphthalene-1- |
| | carbonyloxy)-3-(3,4-dihydroxyphenyl)propanoic acid |
| Emblicanin a | 2-{[2-(3,4-dihydroxyphenyl)-5,7-dihydroxy-4-oxo-4H-chromen- |
| | 3-yl]oxy}-4,5-dihydroxy-6-[(3,4,5-trihydroxybenzoyloxy)methyl] |
| | oxan-3-yl 3,4,5-trihydroxybenzoate |
| Emblicanin a | Remurin B |
| Emblicanin a | Arjuna |
| Emblicanin a | 5-Hydroxyferuloyl-Coa |
| Emblicanin a | Geraniinic Acid B |
| Emblicanin a | Casuariin |
| Emblicanin a | Eugeniin |
| Emblicanin a | Sinapoyl-Coa |
| Emblicanin b | Geraniin |
| Emblicanin a | Feruloyl-Coa |
| Emblicanin a | Balanophotannin A |
| Emblicanin b | 6-(4-{7-[(6-carboxy-3,4,5-trihydroxyoxan-2-yl)oxy]-5-hydroxy-4- |
| | oxo-4H-chromen-2-yl}phenoxy)-5-[(6-carboxy-4,5-dihydroxy-3- |
| | {[3-(4-hydroxyphenyl)prop-2-enoyl]oxy}oxan-2-yl)oxy]-3,4- |
| | dihydroxyoxane-2-carboxylic acid |
| Emblicanin a | Chebulagic Acid |
| Emblicanin b | Eutannin |
| Emblicanin b | Balanophotannin A |
| Emblicanin b | Myricetin 3-(2″,3″-Digalloylrhamnoside) |
| Emblicanin b | Pradimicin C |
| Emblicanin b | 5,5″-Bis- [2,3-Dicarboxy-6,7-Dihydroxy-1-(3′,4′- |
| | Dihydroxyphenyl)-1,2-Dihydronaphthalenel |
| Emblicanin a | Stachyurin |
| Emblicanin a | Punicafolin |
| Emblicanin b | Alnusiin |
| Emblicanin b | Phyllanthusiin D |
| Emblicanin a | Caffeoyl-Coa |

In certain aspects, the compound is chebulinic acid or a compound having a similarity score of at least 95% with chebulinic acid (formula I below). Exemplary compounds having a high similarity score with chebulinic acid are listed in Table 2.

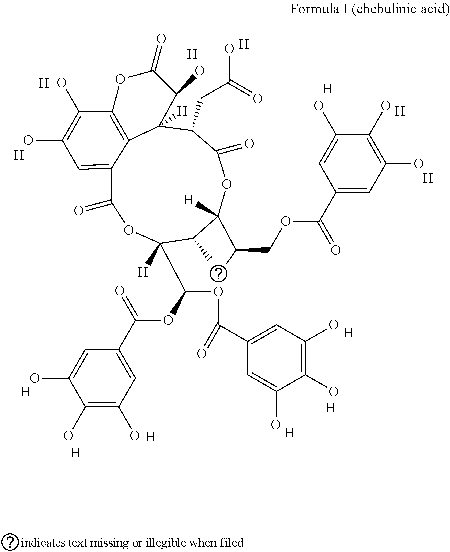

TABLE 2
Compounds having a high similarity score with chebulinic acid.

| Compound | |
|---|---|
| ID from | |
| COCONUT | |
| Database | Structure |
| CNP0285899 | 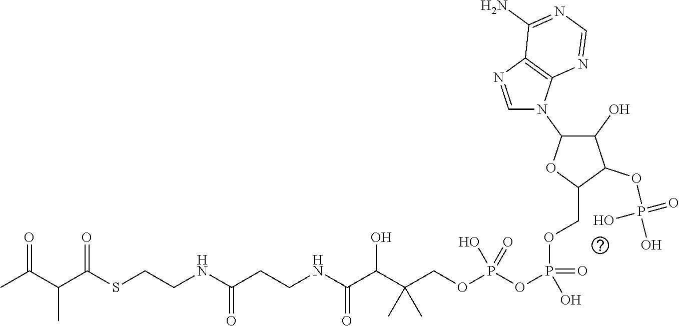 [SMILES: CC(=O)C(C)C(=O)SCCNC(=O)CCNC(=O)C(O)C(C)(C)COP(=O)(O)OP(=O)(O)OCC1OC(n2cnc3c(N)ncnc32)C(O)C1OP(=O)(O)O] |
| CNP0059074 | 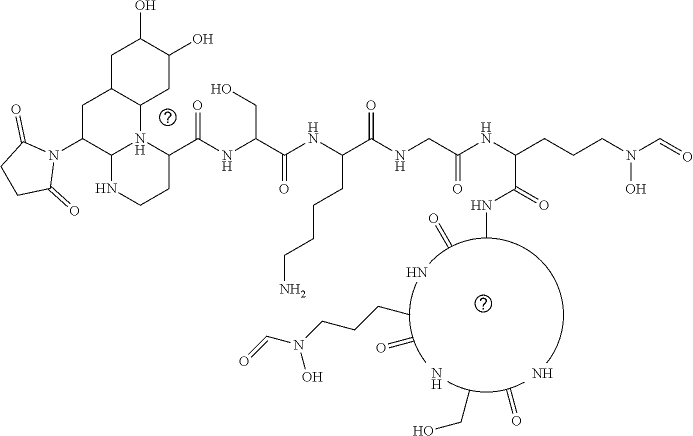 |
| CNP0343660 | 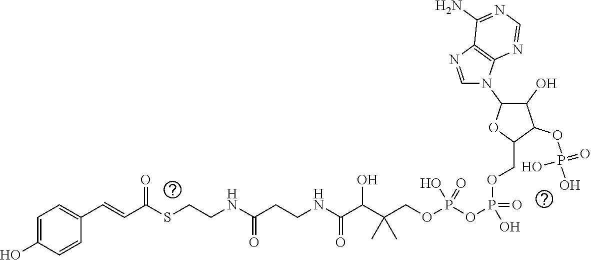 [SMILES: CC(C)(COP(=O)(O)OP(=O)(O)OCC1OC(n2cnc3c(N)ncnc32)C(O)C1OP(=O)(O)O)C(O)C(=O)NCCC(=O)NCCSC(=O)/C=C/c1ccc(O)cc1] |
| CNP0139083 | 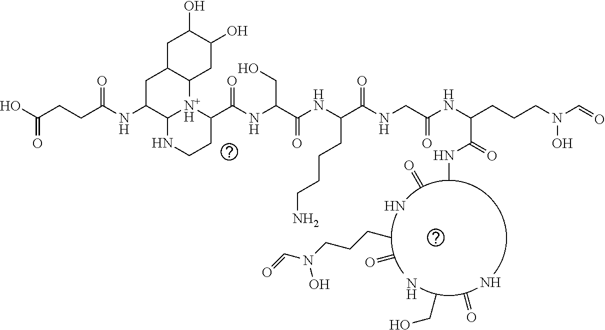 |
| CNP0041318 | 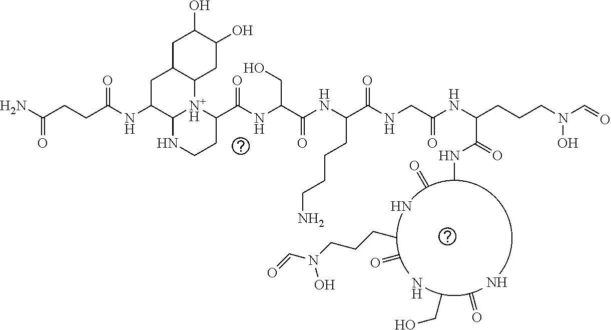 |
| CNP0422373 | 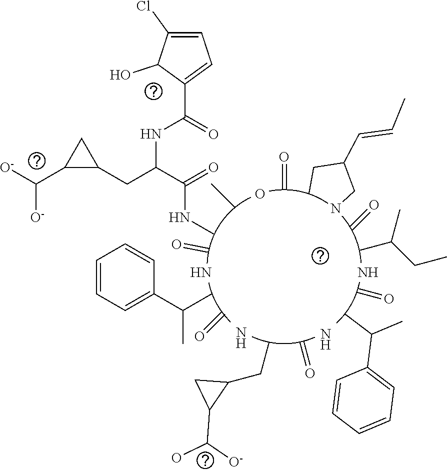 |
| CNP0415802 | 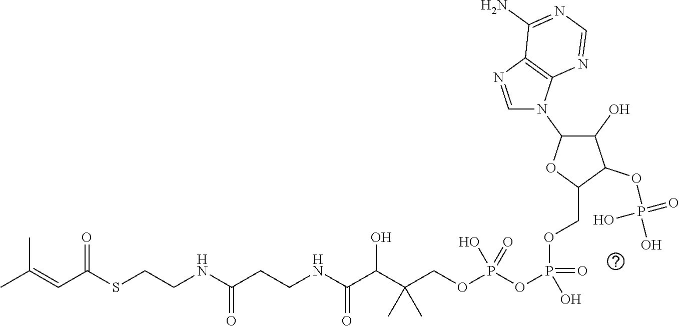 [SMILES: CC(C)=CC(=O)SCCNC(=O)CCNC(=O)C(O)C(C)(C)COP(=O)(O)OP(=O)(O)OCC1OC(n2cnc3c(N)ncnc32)C(O)C1OP(=O)(O)O] |
| CNP0070782 | 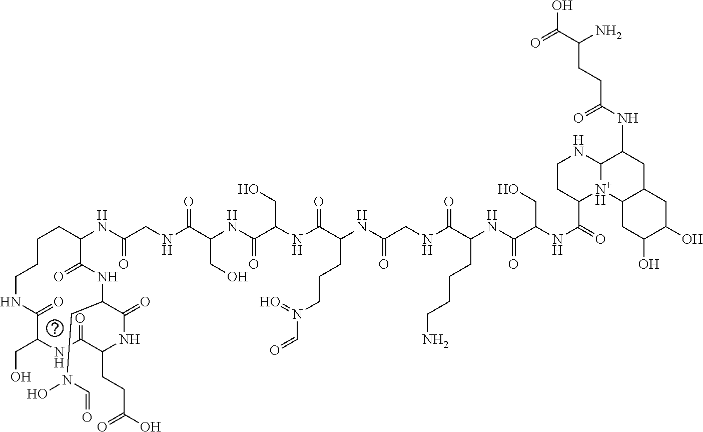 |
| CNP0102046 | 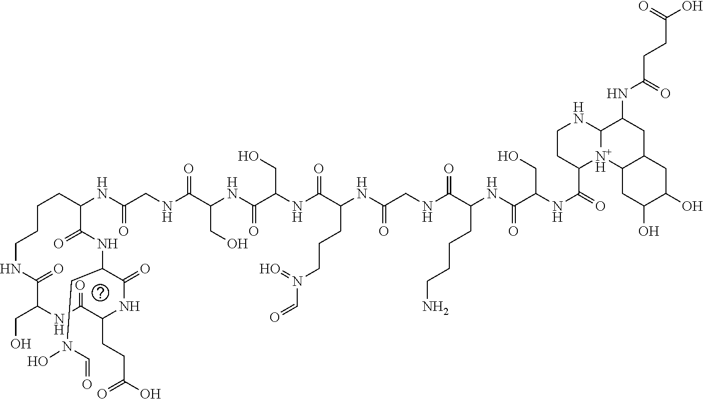 |
| CNP0098207 | 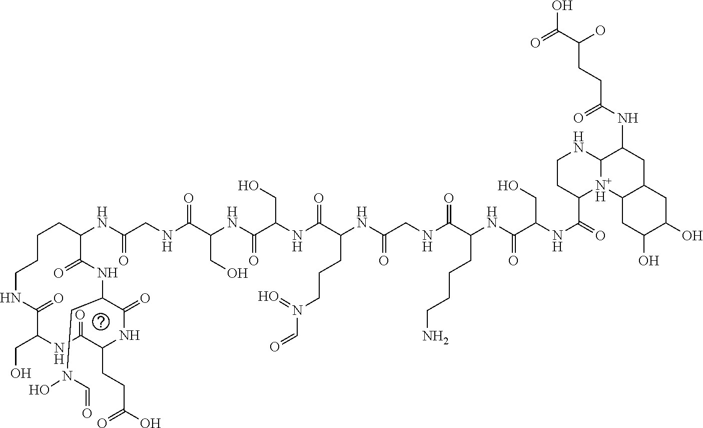 |
| CNP0026197 | 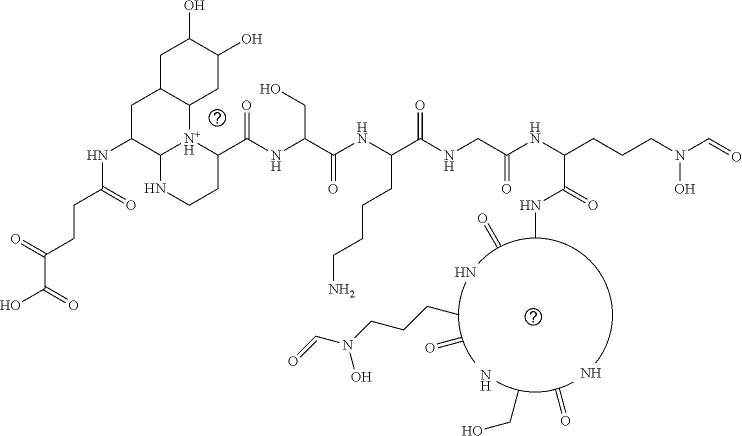 |
| CNP0326396 | 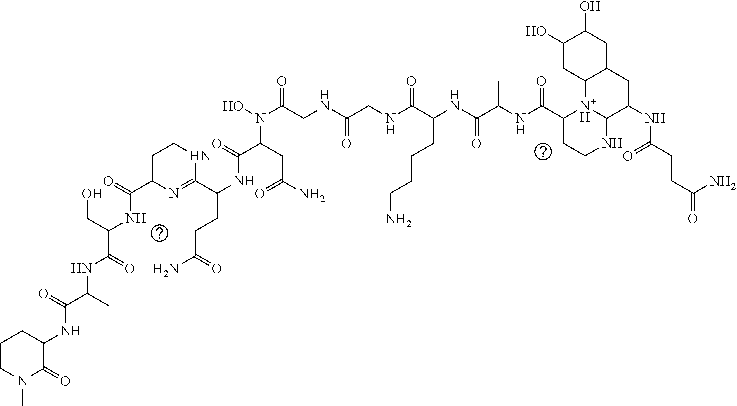 |
| CNP0384812 | 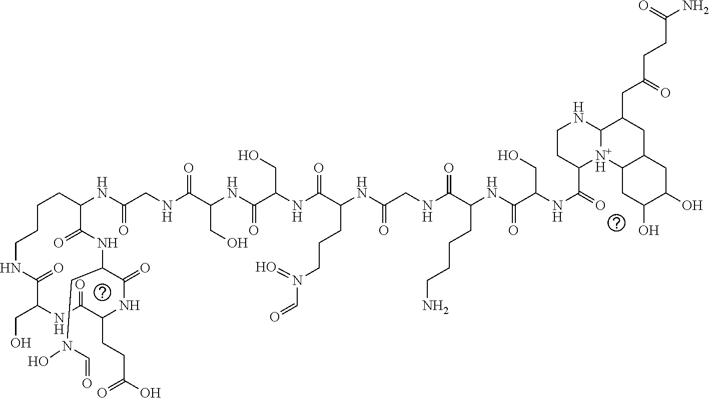 |
| CNP0024232 | 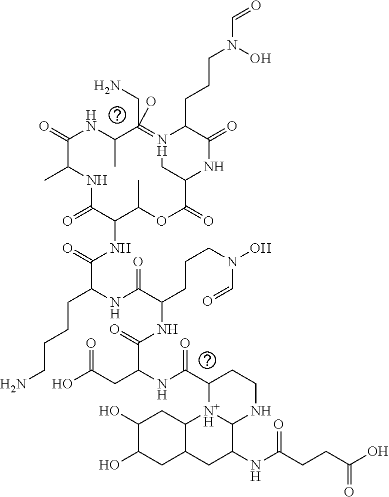 |
| CNP0070582 | 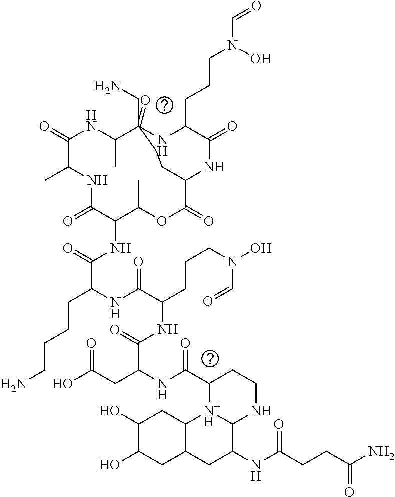 |

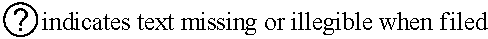

In certain aspects, the compound derived from, constituent of, or purified from an emblica extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, constituent of, or purified from an emblica extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from an emblica extract is a benzene substituted with —CH═CH—(CH₂)ₐ—C(O)OH, wherein a is 0 to 5, and 1 to 5 hydroxy groups. In certain aspects, the compound derived from, constituent of, or purified from an emblica extract is a benzene substituted with —CH═CH—(CH₂)ₐ—C(O)OH, wherein a is 0 to 5, and 1 to 3 hydroxy groups. In certain aspects, the compound derived from, constituent of, or purified from an emblica extract is a benzene substituted with —CH═CH—(CH₂)ₐ—C(O)OH, wherein a is 0, and 1 to 3 hydroxy groups.

In certain aspects, the compound derived from, constituent of, or purified from an emblica extract is gallic acid, vanillic acid, chlorogenic acid, caffeic acid, syringic acid, coumaric aid, quercetin, emblicanin A, emblicanin B, punigluconin, pedunculagin, punicafolin, phyllanemblin, kaempferol, ellagic acid, chebulinic acid, chebulagic acid, punicalagin, or a metabolite of any of the foregoing. In certain aspects, the compound derived from, constituent of, or purified from an emblica extract is ascorbic acid or citric acid.

In certain aspects, the compound derived from, constituent of, or purified from an emblica extract is gallic acid, vanillic acid, chlorogenic acid, caffeic acid, syringic acid, coumaric aid, quercetin, vitamin C, or a metabolite of any of the foregoing.

In certain aspects, the compound derived from, constituent of, or purified from an emblica extract is gallic acid or a compound having a similarity score of at least 95% with gallic acid.

In certain aspects, the active agent in the composition is gallic acid. The active agent may be or comprise an ellagitannin. The active agent may be or comprise emblicanin A, emblicanin B, punigluconin, pedunculagin, and/or chebulinic acid.

In accordance with certain embodiments, the composition may comprise an emblica extract fortified with one or more compound that is a constituent of an emblica extract. The compound constituent of the emblica extract or combination of compounds constituents of the emblica extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from an emblica extract, optionally the effective amount of an active agent of the composition, is from 1 to 100 mg of the, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from an emblica extract.

In certain aspects, the fucus extract is derived from Fucus vesiculosus, Fucus serratus, Fucus, spiralis, or Fucus guiryi. In certain aspects, the fucus extract is derived from Fucus vesiculosus. Such an extract may be derived from any portion of the algae as desired. In preparing such an extract, the Fucus vesiculosus, Fucus serratus, Fucus, spiralis, or Fucus guiryi may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of fucus extract is from 1 to 100 mg of extract, such as 1 mg, 5 mg, 10 mg, 20 mg, 30 mg, 40 mg, 50 mg, 60 mg, 70 mg, 80 mg, 90 mg, or 100 mg of a fucus extract.

In certain aspects, the compound derived from, constituent of, or purified from a fucus extract is a breakdown product of a tannin. In certain aspects, the compound derived from, constituent of, or purified from a fucus extract is an ellagitannin. In certain aspects, the compound derived from, constituent of, or purified from a fucus extract is fucoidan.

In certain aspects, the compound derived from, constituent of, or purified from a fucus extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, constituent of, or purified from a fucus extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from a fucus extract is a benzene substituted with —CH═CH—(CH₂)ₐ—C(O)OH, wherein a is 0 to 5, and 1 to 5 hydroxy groups. In certain aspects, the compound derived from, constituent of, or purified from a fucus extract a benzene substituted with —CH═CH—(CH₂)ₐ—C(O)OH, wherein a is 0 to 5, and 1 to 3 hydroxy groups. In certain aspects, the compound derived from, constituent of, or purified from a fucus extract a benzene substituted with —CH═CH—(CH₂)ₐ—C(O)OH, wherein a is 0, and 1 to 3 hydroxy groups.

In certain aspects, the compound derived from, constituent of, or purified from a fucus extract is gallic acid, vanillic acid, chlorogenic acid, caffeic acid, syringic acid, coumaric aid, or a metabolite of any of the foregoing.

In certain aspects, the compound derived from, constituent of, or purified from a fucus extract is gallic acid.

In certain aspects, the active agent in the composition is gallic acid or a compound having a similarity score of at least 95% with gallic acid. In certain aspects, the active agent in the composition is chebulinic acid or a compound having a similarity score of at least 95% with chebulinic acid (see, e.g., Table 2 above). The active agent may be or comprise an ellagitannin or a compound having a similarity score of at least 95% with an ellagitannin (see, e.g., Table 1 above). The active agent may be or comprise fucoidan or a compound having a similarity score of at least 95% with fucoidan.

In accordance with certain embodiments, the composition may comprise a fucus extract fortified with one or more compound that is a constituent of a fucus extract. The compound constituent of a fucus extract or combination of compounds constituents of a fucus extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from a fucus extract, optionally the effective amount of an active agent of the composition, is from 1 to 100 mg, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from a fucus extract.

In certain aspects, the chebula extract is derived from Terminilia chebula, Terminalia arborea, or Lumnitzera racemose. In certain aspects, the chebula extract is derived from Terminilia chebula. Such an extract may be derived from any portion of the plant as desired. In preparing such an extract, the Terminilia chebula, Terminalia arborea, or Lumnitzera racemose may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of chebula extract is from 1 to 100 mg of extract, such as 1 mg, 5 mg, 10 mg, 20 mg, 30 mg, 40 mg, 50 mg, 60 mg, 70 mg, 80 mg, 90 mg, or 100 mg of a chebula extract.

In certain aspects, the compound derived from, constituent of, or purified from a chebula extract is a breakdown product of a tannin. In certain aspects, the compound derived from, constituent of, or purified from a chebula extract is an ellagitannin. In certain aspects, the compound derived from, constituent of, or purified from a chebula extract is chebulinic acid.

In certain aspects, the compound derived from, constituent of, or purified from a chebula extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, constituent of, or purified from a chebula extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from a chebula extract is a benzene substituted with —CH═CH—(CH₂)ₐ—C(O)OH, wherein a is 0 to 5, and 1 to 5 hydroxy groups. In certain aspects, the compound derived from, constituent of, or purified from a chebula extract a benzene substituted with —CH═CH—(CH₂)ₐ—C(O)OH, wherein a is 0 to 5, and 1 to 3 hydroxy groups. In certain aspects, the compound derived from, constituent of, or purified from a fucus extract a benzene substituted with —CH═CH—(CH₂)ₐ—C(O)OH, wherein a is 0, and 1 to 3 hydroxy groups.

In certain aspects, the compound derived from, constituent of, or purified from a chebula extract is chebulinic acid, ellagic acid, gallic acid, tannic acid, punicalagin, chebulagic acid, or a metabolite of any of the foregoing.

In certain aspects, the compound derived from, constituent of, or purified from a fucus extract is chebulinic acid.

In certain aspects, the active agent in the composition is chebulinic acid or a compound having a similarity score of at least 95% with chebulinic acid (see, e.g., Table 2 above). The active agent may be or comprise an ellagitannin or a compound having a similarity score of at least 95% with an ellagitannin (see, e.g., Table 1 above).

In accordance with certain embodiments, the composition may comprise a chebula extract fortified with one or more compound that is a constituent of a chebula extract. The compound constituent of a chebula extract or combination of compounds constituents of a chebula extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from a chebula extract, optionally the effective amount of an active agent of the composition, is from 1 to 100 mg, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from a chebula extract.

The methods, and any of aspects of the foregoing, may comprise administering a composition comprising an effective amount of two or more of an emblica extract or a compound constituent of an emblica extract, an effective amount of a fucus extract or a compound constituent of a fucus extract, and an effective amount of a chebula extract or a compound constituent of a chebula extract. In accordance with certain embodiments, the two or more of emblica extract or compound constituent of the emblica extract, the fucus extract or compound constituent of the fucus extract, and the chebula extract or compound constituent of the chebula extract may provide synergistic effects in the treatment of the disease or condition.

The compositions disclosed herein may comprise or be fortified with one or more of: an emblica extract or compound constituent of an emblica extract, a fucus extract or compound constituent of a fucus extract, a chebula extract or a compound constituent of a chebula extract, BAMLET 10, BAMLET 50, ferulic acid, quercetin, urolithin A, pterostilbene, acadesine, embelin, EGCG, eriocitrin, gallic acid, gomsin A, lutein, luteolin, NAD, rutin, zeaxanthin, and melatonin.

The methods, and any aspects of the foregoing, may further comprise one or more of the steps: i) identifying a subject in need or treatment; and (ii) providing a compound of the disclosure or a pharmaceutical composition comprising a compound of the disclosure.

In any of the foregoing embodiments, and any aspects of the foregoing, when the term “preventing” is used the term may refers to at least a partial inhibition, for example a 10% inhibition, a 20% inhibition, a 30% inhibition, a 40% inhibition, 50% inhibition, a 60% inhibition, a 70% inhibition, an 80% inhibition, a 90% inhibition, a 95% inhibition or greater than 95% inhibition.

In any of the foregoing embodiments, and any aspects of the foregoing, the compound, or pharmaceutically acceptable form thereof, may be administered alone or as a part of a pharmaceutical composition. The pharmaceutical composition may be formulated by combining a solution of the compound with a pharmaceutically suitable carrier. The solution of the compound may comprise 1 to 1,000 mg of the compound, for example, 10 to 600 mg, 20 to 500 mg, 30 to 200 mg, 50 to 100 mg, or 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, or 1,000 mg of the compound.

The compound and the pharmaceutically suitable carrier may be combined in a ratio of 1:5 to 5:1. The pharmaceutical composition may be formulated to have a concentration of 1 to 1,000 mg/ml of the compound, for example, 10 to 600 mg/ml, 20 to 500 mg/ml, 30 to 200 mg/ml, 50 to 100 mg/ml, or 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 200 mg/ml, 300 mg/ml, 400 mg/ml, 500 mg/ml, 600 mg/ml, 700 mg/ml, 800 mg/ml, 900 mg/ml, or 1,000 mg/ml of the compound.

The pharmaceutical composition may be formulated to have a concentration of 0.01% to 2% of the compound, for example, 0.01%, 0.02%, 0.05%, 0.1%, 0.2%, 0.5%, 1%, or 2% of the compound.

The pharmaceutical composition may be formulated to have a concentration of 2.5 to 50 μM of the compound, for example, 2.5 μM, 5 μM, 10 μM, 25 μM, or 50 μM of the compound. The pharmaceutical composition may be formulated to have a concentration of 50 to 500 μM of the compound, for example, 50 μM, 100 μM, 200 μM, 300 μM, 400 μM, or 500 μM of the compound.

The pharmaceutical composition may be formulated to have a concentration of 2.5 to 50 μM of the compound, for example, 2.5 μg/L, 5 μg/L, 10 μg/L, 25 μg/L, or 50 μg/L of the compound. The pharmaceutical composition may be formulated to have a concentration of 50 to 500 μg/L of the compound, for example, 50 μg/L, 100 μg/L, 200 μg/L, 300 μg/L, 400 μg/L, or 500 μg/L of the compound.

In any of the foregoing embodiments, and any aspects of the foregoing, the compound, or pharmaceutically acceptable form thereof, may be administered alone or as a part of a cosmetic composition. The cosmetic composition may be formulated by combining a solution of the compound with a cosmetically suitable carrier. The solution of the compound may comprise 1 to 1,000 mg of the compound, for example, 10 to 600 mg, 20 to 500 mg, 30 to 200 mg, 50 to 100 mg, or 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, or 1,000 mg of the compound.

The compound and the cosmetically suitable carrier may be combined in a ratio of 1:5 to 5:1. The cosmetic composition may be formulated to have a concentration of 1 to 1,000 mg/ml of the compound, for example, 10 to 600 mg/ml, 20 to 500 mg/ml, 30 to 200 mg/ml, 50 to 100 mg/ml, or 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 200 mg/ml, 300 mg/ml, 400 mg/ml, 500 mg/ml, 600 mg/ml, 700 mg/ml, 800 mg/ml, 900 mg/ml, or 1,000 mg/ml of the compound.

In any of the methods, and any aspects of the foregoing, a compound described herein is in the form of a pharmaceutically acceptable salt, solvate, or hydrate. Such a compound may be formulated as a pharmaceutically acceptable salt, e.g., acid addition salt, and complexes thereof. The preparation of such salts can facilitate the pharmacological use by altering the physical characteristics of the agent without preventing its physiological effect. Examples of useful alterations in physical properties include, but are not limited to, increasing the solubility to facilitate administering higher concentrations of the compound.

In any of the methods, and any aspects of the foregoing, a compound or an extract described herein is administered topically, intravenously, intraperitoneally, parenterally, intramuscularly, orally or via the respiratory tract. In any of the methods, and any aspects of the foregoing, the compound or the extract is administered topically.

The compound or an extract described herein may be administered locally, e.g., at a local site of the disease or condition. The compound or an extract described herein may be formulated for local administration, e.g., to a target site of the disease or condition. The compound or an extract described herein may be administered systemically. Systemic administration may be, e.g., topical, intravenous, intraperitoneal, parenteral, intramuscular, oral, or via the respiratory tract. The compound or an extract described herein may be formulated for systemic administration.

In any of the methods, and any of the aspects of the foregoing, the subject animal is a vertebrate. In any of the methods, and any aspects of the foregoing, the subject is a mammal. In any of the methods, and any aspects of the foregoing, the subject is a human. In any of the methods, and any of aspects of the foregoing, the subject is a non-human mammal, e.g., a rodent, e.g., a mouse.

In some aspects, the subject may be female. In some aspects, the subject may be male. The subject may be characterized as one of the following ethnicity/race: Asian, Black or African American, Hispanic or Latino, white, or multi-racial. The subject may have a Fitzpatrick Scale score of from I to IV. The subject may be of an age less than 1, or between 1-5, 5-10, 10-20, 20-30, 30-40, 40-50, 50-60, or over 60 years.

In any of the methods, and any aspects of the foregoing, the compound or the extract is administered in an effective amount. Suitable effective amounts are described in more detail herein. In any of the methods, and any of the aspects of the foregoing, the compound or the extract is administered in a therapeutically effective amount. In any of the methods, and any aspects of the foregoing, the administering step may comprise administering a single dose of a compound or extract according to a course of treatment (where the dose may contain an effective amount). In any of the methods, and any aspects of the foregoing, the administering step may comprise administering more than one dose of a compound or extract according to a course of treatment (where one or more doses may contain an effective amount). The amount of a compound or extract in each dose administered during a course of treatment is not required to be the same. For example, the administering step may comprise administering at least one loading dose and at least one maintenance dose during a course of treatment. Dosing is described in more details herein.

The compound may be administered as a prophylactic treatment. The compound may be administered as a cosmetic or therapeutic treatment.

In some aspects, at least one wrinkle treated in the subject may comprise a fine line, surface line, or deep furrow. An amount and/or a frequency of administration may be sufficient to decrease an appearance, e.g., severity, of wrinkle in the subject. An amount and/or a frequency of administration may be sufficient to decrease a width of wrinkle in the subject. An amount and/or a frequency of administration may be sufficient to decrease a length of wrinkle in the subject. An amount and/or a frequency of administration may be sufficient to decrease a depth of wrinkle in the subject.

In some aspects, hair loss treated in the subject may comprise total alopecia, partial alopecia, male/female pattern baldness, receding hair line, hair thinning, or a decrease in an amount of hair follicles in growth phase. An amount and/or a frequency of administration may be sufficient to increase an appearance of hair volume or length in the subject. An amount and/or a frequency of administration may be sufficient to increase an amount of hair follicles in growth phase in a subject. An amount and/or frequency may be sufficient to induce hair growth of dormant hair follicles in a subject. An amount and/or a frequency of administration may be sufficient to decrease an appearance of hair loss. An amount and/or a frequency of administration may be sufficient to decrease an amount of hair follicles in a transition or resting phase in a subject. An amount and/or frequency may be sufficient to decrease an amount of dormant hair follicles in a subject.

The compounds disclosed herein may be used in various applications, e.g., cosmetic and/or therapeutic applications. The compounds may be administered in an effective amount for an intended use, e.g., a cosmetic or a therapeutic application. In some embodiments, a composition may comprise a concentration or amount, e.g., an effective amount, of the compound sufficient to have a desired cosmetic effect. In some embodiments, a composition may comprise a concentration or amount, e.g., an effective amount, of the compound sufficient to have a desired therapeutic effect.

An amount and/or frequency of administration may be sufficient to induce mitochondrial biogenesis. An amount and/or a frequency of administration may be sufficient to treat, inhibit, or prevent the progression of at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. An amount and/or frequency of administration may be sufficient to modify a score of a parameter on a qualitative scale, as graded by the subject or a clinical grader. The qualitative scale may comprise the following categories: none (best possible condition), mild, moderate, severe (worst possible condition). The qualitative scale may refer to perceived or actual energy levels and/or vitality.

An amount and/or a frequency of administration may be sufficient to treat, inhibit, or prevent the progression of at least one of scarring (e.g., scar relating to sunburn, bed sore, wound, inflammatory lesion, or burn), skin thickening (e.g. keloid scarring), crack, fissure, heloma, sebum secretion, skin thickening, wrinkle, sun spot, skin tag, dark patch, stretch mark, spider vein, varicose vein, age spot, or pore appearance in the subject. An amount and/or a frequency of administration may be sufficient to treat, inhibit, or prevent the progression of hair loss. An amount and/or a frequency of administration may be sufficient to reduce, inhibit, or prevent the progression of blotchiness or discoloration (e.g., vitiligo or post-inflammatory hyperpigmentation) associated with skin of the subject. An amount and/or a frequency of administration may be sufficient to reduce, inhibit, or prevent the progression of freckles associated with skin of the subject. An amount and/or a frequency of administration may be sufficient to promote firmness, hydration, elasticity, radiance, tone evenness, visual smoothness, or tactile smoothness associated with skin of the subject.

An amount and/or frequency of administration may be sufficient to modify or reduce a score of a parameter on the Griffiths' 10-point scale, for example, as graded by the subject or a clinical grader, according to the following medical definitions (with half-point scores assigned as necessary to accurately describe the skin condition):
0=none (best possible condition)
1 to 3=mild
4 to 6=moderate
7 to 9=severe (worst possible condition).

Exemplary parameters include oily appearance (shine and feel) with 0=no shine, flat/matte appearance and 9=strong shiny/oily appearance; pore appearance with 0=small, tight, barely perceptible pores and 9=large, noticeable pores; radiance with 0=radiant, luminous appearance and 9=dull/matte and or sallow appearance; blotchiness with 0=no blotchiness/clear and 9=blotchy skin appearance; skin tone (color) evenness with 0=even, healthy skin color and 9=uneven, discolored appearance; visual smoothness with 0=smooth, even looking skin texture and 9=rough, uneven looking skin texture; and tactile smoothness with 0=smooth, even feeling skin texture and 9=rough, uneven feeling skin texture. The modification or reduction of score may be by 9 points, 8 points, 7 points, 6 points, 5 points, 4 points, 3 points, 2 points, or 1 point.

In some aspects, administering an effective amount of the compound may limit or inhibit injury to skin integrity of a cutaneous layer of skin in the subject. Administering an effective amount of the compound may prevent an undesired topographical change of the cutaneous layer of skin in the subject. Administering an effective amount of the compound may slow progression from a first topographical, structural, or matrix profile to a second topographical, structural, or matrix profile associated with the cutaneous layer of skin in the subject. In some aspects, the cutaneous layer may pertain to an epidermis (e.g. stratum corneum, stratum lucidum, stratum granulosum, stratum spinosum, or stratum basalis), a basement membrane, a dermal-epidermal junction, a dermis (e.g. papillary dermis, reticular dermis, or any vasculature comprising the dermis), or subcutis (e.g. subcutaneous fat layer) of the subject.

In some aspects, administering an effective amount of the compound may limit or inhibit at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. For example, the effective amount of the compound may slow progression of the at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. In some embodiments, administering an effective amount of the compound may promote mitochondrial biogenesis, mitochondrial function, and/or an increase in energy level or vitality.

In some aspects, the skin of the subject may be substantially non-diseased. The skin of the subject may be substantially uninjured. The skin of the subject may be substantially free of inflammatory lesions. The skin of the subject may be substantially free of a mild to severe crack, fissure, or wrinkle. The skin of the subject may be substantially free of a mild to severe scar (e.g., scar relating to sunburn, bed sore, wound, inflammatory lesion, or burn) or stretch mark. The skin of the subject may be substantially free of a mild to severe sun spot, dark patch, or age spot. The skin of the subject may be substantially free of a mild to severe heloma, skin thickening, skin tag, or keloid scar. The skin of the subject may be substantially free of an appearance of a varicose vein or a spider vein. The skin of the subject may be substantially free of a mild to severe appearance of pores. The skin of the subject may be substantially free of mild to severe cellulitis.

In some aspects, the compound may be administered prior to onset of the disease or condition in the subject. The compound may be administered during incidence of the disease or condition in the subject. The compound may be administered subsequent to at least partial reduction of the disease or condition in the subject. The compound may be administered in response to a trigger or warning sign of a mitochondrial dysfunction or aging-associated condition, e.g., aging, premature aging, habitual sleep conditions, habitual sleep position, habitual facial expression, weight loss, ultraviolet (UV) light exposure, treatment with a chemical or therapeutic agent, e.g., chemotherapy and/or radiation therapy, smoking, dehydration, or immersion. The subject may be predisposed for a mitochondrial dysfunction condition, e.g., based on age, race, skin type, eye color, habit, or heredity. A method may further comprise determining whether the subject is in need of treatment. The composition comprising the compound may additionally comprise a moisturizing agent, deodorizing agent, scent, colorant, insect repellant, cleansing agent, or UV-blocking agent. The composition may include microspheres or microcapsules.

The composition may be formulated for immediate release or extended release. The composition may be formulated for controlled or sustained release. For example, the composition may be formulated for sustained release over a period of 6 hours, 12 hours, 18 hours, 24 hours, 36 hours, 48 hours, or more.

The subject may be characterized as having normal mitochondrial function. The subject may be characterized as having reduced mitochondrial function. The subject may be characterized as experiencing premature aging or a symptom of premature aging. In certain embodiments, the subject may be characterized by one or more of: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, increased epidermal hyperplasia, acanthosis, hyperkeratosis, increased expression of at least one gene selected from the group consisting of: NF-κB, COX-2, INF-β1, CCL5, MMP1, MMP2, MMP9, MMP13, IGF1R, VEGF, and MRPS5, decreased expression of at least one of gene selected from the ground consisting of TIMP1, KLOTHO, COL1A1, MTCO2, TFAM, and VDAC, and increased inflammatory infiltrate in skin. The subject may be characterized as having decreased expression at least one protein selected from PGC-1a, TFAM, NRF-1, and COXII.

In some aspects, a method may further comprise administering a second amount of the compound to the subject. The second amount may be administered as a second dose of the same formulation. The second amount may be administered in another formulation. The second amount may be administered in another formulation by the same route of administration, e.g., a topical solution or oil with a shampoo, conditioner, spray, cream, gel, body wash, soap, or lotion. The second amount may be administered by another route of administration, e.g., each amount may independently be administered topically, parenterally, or enterally.

In some aspects, a method may further comprise administering a second compound to the subject. The second compound may be a compound that is a constituent of the same extract. The second compound may be a compound that is a constituent of another extract. The second compound may be a compound having a similarity score of at least 95% with the first compound. The second compound may be administered in the same formulation. The second compound may be administered in another formulation.

The compound may be administered as part of a combination therapy. The method may further comprise administering a second treatment in combination with the compound. The compound may be administered for a period of time prior to initiating the second treatment. The compound may be administered concurrently with the second treatment. The compound may be administered for a period of time subsequent to ceasing the second treatment. The second treatment may be administered via an alternate mode of administration. The second treatment may be a cosmetic and/or therapeutic treatment.

The compound may be administered in combination with a second agent, e.g., cosmetic or therapeutic agent, approved to treat or commonly used to treat the disease or condition or a symptom thereof.

The compound may be administered in combination with caffeine, B vitamins (e.g., B1, B2, B3, B5, B6, B8, B9 and/or B12), vitamin C, iron, magnesium, and/or zinc.

The compound may be administered in combination with UV-blocking agent, moisturizer, sunscreen, wrinkle cream, retinoid, alpha-hydroxy acid, beta-hydroxy acid, squalene, antioxidant, tretinoin, glycosaminoglycan (GAG), tactic acid, malic acid, citric acid, tartaric acid, hydroquinone, kojic acid, L-ascorbic acid, licorice extract, N-acetylglucosamine, niacinamide, squalene, soy, dermal filler or injection, e.g. hyaluronic acid or calcium hydroxylapatite, botulinum toxin, laser resurfacing procedure, ultrasound therapy, chemical peel, e.g. glycolic acid peel, trichloroacetic acid or salicylic acid, or dermabrasion procedure.

The compound may be administered in combination with an antioxidant. Exemplary antioxidants include CoQ10, vitamin C, vitamin E, carotenoids, e.g., beta-carotene, minerals, e.g., selenium and manganese, glutathione, lipoic acid, flavonoids, betaflavinoids, phenols, polyphenols, phytoestrogens, mitoquinol mesylate, and ubiquinone.

The fucus compound may be administered in combination with an emblica extract or a compound constituent of an emblica extract. The fucus compound may be administered in combination with a chebula extract or a compound constituent of an chebula extract. The emblica compound may be administered with a fucus extract or a compound constituent of a fucus extract. The emblica compound may be administered with a chebula extract or a compound constituent of a chebula extract. In accordance with certain embodiments, the combination of two or more of an emblica compound, a fucus compound, and a chebula compound may provide synergistic effects in the treatment of the disease or condition.

In accordance with one or more embodiments, an effective amount of the compound may be administered to a face of a subject. In accordance with one or more embodiments, the compound may be administered to the scalp of the subject. In accordance with one or more embodiments, the compound may be administered to the body of the subject. For example, the compound may be applied to one or more of the more of the forehead, eye region, neck, scalp, head, shoulder, arm, hands, leg, underarm, torso, chest, feet, knee, ankle, back, buttock, or genitals of the subject.

### Dosage and Administration

In accordance with the methods, the compounds of the disclosure are administered to the subject (or are contacted with cells of the subject) in an effective amount.

In certain embodiments, therapeutically the effective amount of a compound of the disclosure ranges from about 0.05 mg/kg/day to about 50 mg/kg/day. In certain embodiments, the effective amount ranges from about 0.05 mg/kg/day to about 40 mg/kg/day. In certain embodiments, the effective amount ranges from about 0.05 mg/kg/day to about 30 mg/kg/day. In certain embodiments, the effective amount ranges from about 0.05 mg/kg/day to about 20 mg/kg/day. In certain embodiments, the effective amount ranges from about 0.05 mg/kg/day to about 10 mg/kg/day. In certain embodiments, the effective amount ranges from about 0.05 mg/kg/day to about 8 mg/kg/day. In certain embodiments, the effective amount ranges from about 0.05 mg/kg/day to about 6 mg/kg/day. In certain embodiments, the effective amount ranges from about 0.05 mg/kg/day to about 4 mg/kg/day. In certain embodiments, the effective amount ranges from about 0.05 mg/kg/day to about 3 mg/kg/day. In certain embodiments, the effective amount ranges from about 0.05 mg/kg/day to about 2 mg/kg/day. In certain embodiments, the effective amount ranges from about 0.05 mg/kg/day to about 1 mg/kg/day. In certain embodiments, the effective amount ranges from about 0.05 mg/kg/day to about 0.8 mg/kg/day. In certain embodiments, the effective amount ranges from about 0.05 mg/kg/day to about 0.6 mg/kg/day. In certain embodiments, the effective amount ranges from about 0.05 mg/kg/day to about 0.4 mg/kg/day. In certain embodiment, the amounts per day described above are administered according to a course of treatment and may be administered in a single dose or in more than 1 dose per day. The amounts per day described above may be administered according to a course of treatment and administered in one dose (q.d.) or two doses each day (b.i.d.), wherein the amount of the compound of the disclosure in each dose need not be the same.

In certain embodiments, each dose is administered according to a course of treatment. As used herein, the term “dose” refers to an amount of a compound of the disclosure administered at a given time point according to a course of treatment. For example, if a course of treatment for a compound of the disclosure is b.i.d (2 times/administrations per day) for 7 days, the two administrations on each of days 1-7 would each comprise administering a dose of a compound of the disclosure (for 2 doses each day). In certain embodiments, a dose is administered q.d. (1 time/administration per day) according to a course of treatment. In certain embodiments, a dose is administered b.i.d. according to a course of treatment. In certain embodiments, a dose is administered t.i.d. (three times/administrations per day) according to a course of treatment.

When 2 or more doses are administered on a given day according to a course of treatment, each dose administered according to the course of treatment may contain the same amount of a compound of the disclosure or one or more of doses administered according to the course of treatment may contain a greater or lesser amount of a compound of the disclosure as compared to another dose administered according to the course of treatment. For example, if a course of treatment for a compound of the disclosure is b.i.d for 7 days, the first dose administered on day 1 may contain a first amount (i.e., 2 mg/kg) and the second dose administered on day 1 may contain a second amount (i.e., 0.5 mg/kg). As another example, if a course of treatment for a compound of the disclosure is b.i.d for 7 days, the first dose administered on day 1 may contain a first amount (i.e., 2 mg/kg), the second dose administered on day 1 may contain a second amount (i.e., 0.5 mg/kg), the two doses administered on each of days 2-4 may contain the second amount, and the two doses administered on each of days 5-7 may contain a third amount (i.e., 1 mg/kg).

A dose may be further divided into a sub-dose. Any given dose may be delivered in a single unit dose form or more than one unit dose form. For example, a dose when given by IV administration may be provided as a single IV infusion (i.e., a single 5 mg/kg IV infusion) or as two or more IV infusions administered one after the other (i.e., two 2.5 mg/kg IV infusions). Further, a sub-dose might be, for example, a number of discrete loosely spaced administrations, such as multiple inhalations from an insufflator, by application of a plurality of drops into the eye, or multiple tablets for oral administration.

In certain embodiments, more than one dose of a compound of the disclosure is administered during a course of treatment. Therefore, in the methods described herein, the methods may comprise the administration of multiple doses during the course of treatment. In certain embodiments, the course of treatment may range from months to years. In certain embodiments, the course of treatment may range from 2 days to 1 month, from 2 days to 3 weeks, from 2 days to 2 weeks, or from 2 days to 1 week. In certain embodiments, the course of treatment may range from 1 year to 20 years or longer. In certain embodiments, a dose is delivered at least 1 time per day (i.e., 1 to 3 times) during the course of treatment. In certain embodiments, a dose is not administered every day during the course of treatment (for example, a dose is be administered at least 1 timer per day every other day, every third day, every week, or every month during the course of treatment). Furthermore, the amount of a compound of the disclosure in each dose need not be the same as discussed above. In certain embodiments, of the foregoing, one or more doses, or all of the doses, contain an effective amount of a compound of the disclosure.

In one embodiment, a course of treatment may comprise administering at least one dose as a loading dose and at least one dose as a maintenance dose, wherein the loading dose contains a greater amount of a compound of the disclosure as compared to the maintenance dose (such as, but not limited to, 2 to 10 times higher). In one aspect of this embodiment, the loading dose is administered initially, either as a single administration or more than one administration, followed by administration of one or more maintenance doses through the remaining course of treatment. For example, for a course of treatment that is q.d. every week for 10 years, a loading dose of 3 mg/kg may be administered as the first dose on week 1 of the course of treatment, followed by maintenance doses of 0.5 mg/kg every week for the remainder of the course of treatment. Furthermore, a loading dose may be given as a dose that is not the first dose administered during a course of treatment. For example, a loading dose may be administered as the first dose on week and as a dose on one or more additional weeks (for example, weeks 10 and 20).

In one embodiment, a course of treatment may comprise administering a first dose formulated in a first composition and administering at least one second or subsequent dose formulated in a second composition. The first composition and the second composition may be the same formulation. The first composition and the second composition may be different formulations.

Pharmaceutical, Cosmetic, and/or Dietary Compositions

Pharmaceutical compositions are provided that comprise an amount, e.g., an effective amount, of a compound of the disclosure. In one embodiment, such pharmaceutical compositions contain a therapeutically effective amount of a compound of the disclosure. In addition, other active agents may be included in such pharmaceutical compositions. Additional active agents to be included may be selected based on the disease or condition to be treated.

The pharmaceutical compositions disclosed may comprise one or more compound of the disclosure, alone or in combination with additional active agents, in combination with a pharmaceutically acceptable carrier and/or excipient and/or in combination with a cosmetically acceptable carrier and/or excipient. Such pharmaceutical compositions may be used in the manufacture of a medicament for use in the methods described herein. The compounds of the disclosure are useful in both free form and in the pharmaceutically acceptable forms, such as pharmaceutically acceptable salts.

The pharmaceutically acceptable carriers and/or excipients and/or cosmetically acceptable carriers and/or excipients are well-known to those who are skilled in the art. The choice of carrier and/or excipient will be determined in part by the particular compound(s), as well as by the particular method used to administer the compound composition. Accordingly, there is a wide variety of suitable formulations of the composition of the disclosure. The following methods and excipients are merely exemplary and are in no way limiting. Suitable carriers and excipients include solvents such as water, alcohol, and propylene glycol, solid absorbants and diluents, surface active agents, suspending agent, tableting binders, lubricants, flavors, and coloring agents. The pharmaceutically and/or cosmetically acceptable carriers can include polymers and polymer matrices. Examples of acceptable carriers include carboxymethyl cellulose, crystalline cellulose, glycerin, gum arabic, lactose, magnesium stearate, methyl cellulose, powders, saline, sodium alginate, sucrose, starch, talc and water, among others. Typically, the acceptable carrier is chemically inert to the active agents in the composition and has no detrimental side effects or toxicity under the conditions of use.

Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N+R′R″R′″R″″Y—, in which the R radicals are identical or different optionally hydroxylated hydrocarbon radicals and Y— is an anion of a strong acid, such as halide, sulfate and sulfonate anions; cetyltrimethylammonium bromide is one of the cationic surfactants which can be used, amine salts of formula N+R′R″R″′, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Exemplary pharmaceutically acceptable carriers and/or excipients and/or cosmetically acceptable carriers and/or excipients include water, silica, glycerin, dimethicone, butylene glycol, pentylene glycol, ethoxydiglycol, polyacrylate-13, pentapeptide-34 trifluoroacetate, polyisobutene, lysolecithin, sclerotium gum, pullulan, polysorbate 20, diethylhexyl syringylidenemalonate, caprylyl glycol, glyceryl stearate, PEG-100 stearate, cetearyl alcohol, butyrospermum parkii (shea) butter, acetyl tetrapeptide-2, betaine, melanin, tocopheryl acetate, tocopherol, hydroxyacetophenone, caprylic/capric triglyceride, batyl alcohol, C12-15 alkyl benzoate, panthenol, ceteareth-20, xanthan gum, ethylhexylglycerin, disodium EDTA, propanediol, caprylyl glycol, potassium sorbate, sorbic acid, and phenoxyethanol. The compounds of the disclosure and pharmaceutical compositions containing such compounds as described in the instant disclosure can be administered by any conventional method available for use in conjunction with pharmaceuticals, either as individual therapeutic agents or in combination with additional therapeutic agents.

In one embodiment, the compounds of the disclosure are administered in an effective amount, whether alone or as a part of a pharmaceutical composition. The effective amount and the dosage administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

In these pharmaceutical or cosmetic compositions, the compound(s) of the disclosure will ordinarily be present in an amount of about 0.5-95% weight based on the total weight of the composition, or about 0.1-99.9% weight based on the total weight of the composition. Multiple dosage forms may be administered as part of a single treatment.

The active agent can be administered enterally in solid dosage forms, such as capsules, tablets, and powders, or in liquid dosage forms, such as milk, elixirs, syrups and suspensions. It can also be administered parenterally, in sterile liquid dosage forms. The compound(s) of the disclosure can also be administered intranasally (nose drops) or by inhalation via the pulmonary system, such as by propellant based metered dose inhalers or dry powders inhalation devices. Other dosage forms include topical administration, such as administration transdermally, via patch mechanism or ointment.

Formulations suitable for enteral or oral administration may be liquid solutions, such as an effective amount of the compound(s) dissolved in diluents, such as milk, water, saline, buffered solutions, infant formula, other suitable carriers, or combinations thereof. Formulations suitable for enteral or oral administration of the compounds of the disclosure are known in the art as exemplified by: Shaji, et al., Indian J Pharm Sci. 2008 May-June; 70(3): 269-277; Bruno, et al., Ther Deliv. 2013 November; 4(11): 1443-1467; Ibrahim, et al., DARU Journal of Pharmaceutical Sciences, 2020, 28, 403-416. The compound(s) can then be mixed to the diluent just prior to administration. In an alternate embodiment, formulations suitable for enteral or oral administration may be capsules, sachets, tablets, lozenges, and troches. In each embodiment, the formulation may contain a predetermined amount of the compound(s) of the disclosure, as solids or granules, powders, suspensions and suitable emulsions. Liquid formulations may include diluents, such as water and alcohols, for example, ethanol, benzyl alcohol, propylene glycol, glycerin, and the polyethylene alcohols, either with or without the addition of an acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers.

Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) can be administered in a physiologically acceptable diluent in an acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of an acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl .beta.-aminopropionates, and 2-alkylimidazoline quaternary ammonium salts, and (e) mixtures thereof.

The parenteral formulations typically contain from about 0.5% to about 50% by weight of the compound(s) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The compound(s) of the disclosure can be formulated into aerosol formulations to be administered via nasal or pulmonary inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

The compound(s) of the disclosure, alone or in combination with other suitable components, may be administered in an aqueous solution as a nasal or pulmonary spray and may be dispensed in spray form by a variety of methods known to those skilled in the art. Systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof.

Nasal and pulmonary solutions of the disclosure may typically comprise the drug or drug to be delivered, optionally formulated with a surface-active agent, such as a nonionic surfactant (e.g., polysorbate-80), and one or more buffers. In some embodiments, the nasal spray solution further comprises a propellant. The pH of the nasal spray solution is optionally between about pH 3.0 and 6.0, or 4.5+/−0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm. mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonaryly respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically and/or cosmetically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about 1/3 to 3, more typically 1/2 to 2, and most often 3/4 to 1.7.

The compound(s) of the disclosure may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the active agent.

The compounds of the disclosure may be formulated in a nanoparticle-based delivery carrier. The nanoparticle delivery systems disclosed herein may offer benefits in mitochondria-targeted delivery and improving the therapeutic ability of the compounds. Nanoparticle formulations have been shown to effectively transport drug molecules in their original form, solubilize hydrophobic drug molecules, enhance half-life of molecules, and decrease side effects and immunogenicity attributable to the molecules. The nanoparticle delivery carriers disclosed herein may be selected or designed to provide enhanced skin penetration, higher stability, site specific targeting, e.g., efficiently deliver cargo inside the mitochondrial matrix, high entrapment efficiency, and/or time-controlled, e.g., delayed or sustained, release of the compounds. Properties of the nanoparticle delivery carrier that may be designed to effectively deliver compounds of the formulation to a target site of a subject include surface chemistry, coating, structure, size, ability to aggregate, and solubility. Exemplary nanomaterials are described in “Nanotherapeutic Approaches to Target Mitochondria in Cancer,” (Mani, 2021) and “Role of Nanotechnology in Cosmeceuticals: A Review of Recent Advances,” (Kaul, 2018), each of which is incorporated herein by reference in its entirety for all purposes.

The nanoparticle delivery carrier may comprise and/or be functionalized with carbon-based nanomaterials, liposomal delivery vehicles, polymeric nanocarriers, micelles, dendrimers, lipophilic cations, solid-lipid nanoparticles (SLN), peptide-based nanomaterials, nanostructured lipid carriers (NLC), niosomes, nanoemulsions, metal nanoparticles, nanospheres, polymerosomes, cubosomes, and combinations thereof. The delivery carrier may be designed to for mitochondrial targeting or specific cell type targeting. Exemplary mitochondrial targeting agents include antibodies, polymeric functional moieties, such as PEG, lipophilic cations, such as triphenylphosphine (TPP), and peptides, such as mitochondrial penetrating peptides (MPP). The delivery carrier may be formed of a targeting moiety, for example, encapsulating or conjugated with the compounds disclosed herein, and/or the delivery carrier may be functionalized with a surface targeting moiety. The nanoparticle carrier may be dimensioned to have an average size of about 10 to 5000 nm, for example 10 to 50 nm, 10 to 100 nm, 50 to 500 nm, 50 to 100 nm, 100 to 500 nm, 500 to 1000 nm, or 1000 nm to 5000 nm, which can be selected based on the target tissue, compound to be delivered, and other properties of the nanomaterial.

Exemplary carbon-based nanomaterials include carbon dots (C-dots or CD), carbon nanotubes (CNT), graphene derivatives, nanodiamonds (ND), quantum dots (QD), and magnetic nanoparticles (MNP). Carbon nanoparticles may be formed into different shapes including, for example, spherical, elliptical, tube, horn-shaped, and combinations thereof.

CNTs are graphene sheets formed into cylindrical tubes. CNTs have demonstrated low toxicity profile, good biocompatibility, and targeted accumulation. CNTs of the disclosure may be single-walled carbon nanotubes (SWCNT) and/or multi-walled carbon nanotubes (MWCNT). SWCNTs have a smaller diameter, in the range of 1 to 10 nm. MWCNTs have a larger diameter, in the range of 2 to 50 nm. CNTs may be functionalized with a targeting sequence. For instance, CNTs may be functionalized to target mitochondria and/or a specific cell type. Graphene derivatives also include two-dimensional carbon allotropes. Graphene derivatives may be designed to exhibit specific physicochemical properties, such as a high surface area and selected multifaceted surface properties. Graphene derivatives may be functionalized, e.g., surface functionalized, for targeted delivery to mitochondria and/or a specific cell type. CNTs have been used successfully in hair colorants and cosmetic hair care formulations.

Nanodiamonds have been shown to provide high affinity to biomolecules, biocompatibility, and non- or low-cytotoxicity. Nanodiamonds, quantum dots, and magnetic nanoparticles may be conjugated with the compounds disclosed herein for targeted delivery. Certain MNP may be designed to encapsulate the compounds disclosed herein. QDs are generally formed of a semiconductive core layered by a shell designed to provide selected physical and chemical characteristics. MNPs are formed of a magnetic core, such as an iron oxide material core, and surface coating designed to improve stability and biocompatibility in physiological environments. Thus, MNPs and QDs are highly adaptable for their selected use.

Liposome based delivery vehicles are typically formed of enclosed spherical vesicles composed of a lipid bilayer with an internal bilayer and internal aqueous core region. The liposome may have a unilamellar or multilamellar structure. Liposomes may be designed to have a surface chemistry effective for targeted delivery and/or controlled release delivery. For instance, engineered liposomes have shown improved cellular update and accumulation of a delivery compound in the mitochondria. Antioxidants, such as carotenoids, CoQ10, lycopene and agents such as vitamin A, E, and K, may be incorporated into liposomes to amplify physical and chemical stability. Liposomes may be formulated with phosphatidylcholine to provide moisturizing properties to skin and hair care products. Vegetable phospholipids and soya phospholipids may be used with topical formulations for their high content of esterified essential fatty acids. For example, when applied with an active agent, the barrier function of the skin is increased and water loss is decreased. It has been shown that certain liposomes have an effect on wrinkle reduction, decreasing efflorescence in acne treatment, and increasing skin smoothness.

Niosomes are vesicles having a bilayer structure composed of self-assembled hydrated nonionic surfactants. Niosomes may have cholesterol incorporated into their lipids or may be free of cholesterol. Niosomes may be formulated as multilamellar or unilamellar structures encapsulating the compounds disclosed herein by a membrane formed when the surfactant macromolecules are organized as a bilayer. Exemplary nonionic surfactants include spans, tweens, brijs, alkyl amides, sorbitan ester, crown ester, polyoxyethylene alkyl ether, and steroid-linked surfactants. Niosomes may encapsulate the compounds disclosed herein, providing prolonged systemic circulation and enhanced penetration into target tissue. Niosomes in cosmetic and skin care applications provide skin penetration, increased stability of entrapped ingredients, and improved bioavailability of typically poorly adsorbed compounds. Niosomes may be designed for a target application by controlling the nature and structure of surfactants, membrane composition, and temperature of hydration, which influences size and shape of the particle. Specialized niosomes called proniosomes may be used. Proniosomes are nonionic based surfactant vesicles which are hydrated immediately before use to yield aqueous noisome dispersions. To further enhance drug delivery, niosomes and proniosomes may be combined in a formulation.

Polymeric nanocarriers are generally formed of biodegradable polymers. The polymeric nanocarriers may be reservoir type (nanocapsules in which compounds are dissolved/distributed in the core of the polymer), matrix type (nanospheres, in which compounds are entrapped in the polymer matrix), and combinations thereof. Polymeric nanocarriers may offer the benefits of low toxicity, easy modification (for example, for targeted delivery), high drug loading capacity, small size, good aqueous solubility, and biocompatibility. Exemplary polymeric nanocarriers for mitochondrial targeting include hydrophilic block polymer, such as polyethylene glycol (PEG), poly E-caprolactone (PCL). Other nanoparticles disclosed herein may be modified for mitochondrial targeting, such as by including surface hydrophilic block polymers (e.g., PEGylation). Polymerosomes are artificial vesicles formed of self-assembling block copolymer amphiphiles. Polymerosomes typically have a hydrophilic core and lipophilic bilayer. Polymerosomes are highly customizable. Drug encapsulation and release capabilities may be controlled by forming the polymerosome with block copolymers that are biodegradable and/or responsive to stimuli. The composition and molecular weight of the polymerosome may be selected to control properties, such as, response to stimuli, membrane thickness, permeability, flexibility, and size (polymerosomes may be designed to have a radius from 50 nm to 5000 nm or more). Polymerosomes provide benefits, such as, improved skin elasticity and skin cell activation energy enhancement.

Cubosomes are nanostructured particles formed from self-assembled liquid crystalline particles of aqueous lipids and surfactants. Cubosomes are formed of a bicontinuous liquid phase, enclosing two separate vesicles of aqueous formulations divided by a surfactant-controlled bilayer in a strongly packed structure. Cubosomes may be designed as a honeycombed structure having more than two separate vesicles. Release of each vesicle may be separately controlled. Cubosomes may provide benefits, such as, providing controlled and/or targeted release of the compounds, possessing lipid biodegradability, and having a high internal surface area with different drug-loading modalities.

Micelles are colloidal aggregates that are generally amphiphilic in nature, having a hydrophilic head and hydrophobic tail. The size and shape of micelle nanoparticles may be selected by varying the solution's isotonic strength, pH, temperature, and the nature of the amphiphilic molecule. Micelles may be utilized to improve uptake of the compounds disclosed herein in mitochondria. For instance, micelle formulations have been found to improve bioavailability of low absorption compounds, prevent mitochondrial swelling (indicating less mitochondrial permeability transition pore (mPTP) opening and prevention of injury), and protect cells from nitrosative stress, “Curcumin Micelles Improve Mitochondrial Function in Neuronal PC12 Cells and Brains of NMRI Mice—Impact on Bioavailability” (Hagl, 2015) (incorporated herein by reference in its entirety for all purposes). Micelles may be functionalized with a targeting group, such as mitochondrial targeting TPP, MPP, or PEGyltaion. In some embodiments, micelle nanoparticles may be polymeric micelles.

Dendrimers are hyperbranched macromolecules which may be formed of sugars, amino acids, and/or nucleotides. Dendrimers are generally formed of a central core, repeated branches, and diverse peripheral groups. The peripheral groups may be designed or functionalized for a target application, such as targeted delivery. Benefits of dendrimers include the ability to provide drug encapsulation, high aqueous solubility, high retention time, biodegradability, specificity, low toxicity, and surface modification capabilities that may provide properties such as monodispersity, polyvalence, and stability. Dendrimers may be functionalized with a targeting group, such as TPP, MPP, or PEGylation. Dendrimers may be formulated to encapsulate the compound or conjugate to the compound.

Lipophilic cations are positively charged ions that can penetrate the plasma and mitochondrial membranes. The lipophilic cations tend to accumulate in the mitochondria. Thus, lipophilic cations, such as TPP, dequalinum, and rhodamine 123, may be utilized for mitochondrial-targeted therapeutic delivery. Delocalized lipophilic cations (DLC) have a strong mitochondrial targeting ability to cross the membrane and drive specific aggregation of attached moieties within the mitochondria of the cells. Thus, DLCs may be utilized as nanoparticle carriers and/or as surface modifications for targeted delivery. For instance, the compositions disclosed herein may be conjugated to a DLC, such as TPP, or encapsulated in another carrier having a TPP surface functionalization for targeted delivery.

Solid-lipid nanoparticles are sub-micron colloidal carriers in the range of 50 to 1000 nm, for example 50 to 500 nm or 50 to 100 nm. SLNs are generally formed of physiological lipid disseminated in water or a liquid surfactant solution having an oil-based or lipoidal core. SLNs may be prepared from complex glyceride mixtures, purified triglycerides, and waxes having phospholipid hydrophobic chains in the fat matrix. SLNs provide benefits such as small size, large surface area, high drug loading capacity, and the contact of phases at the interface. SLNs may be designed to provide controlled or sustained release of the compound. In cosmetics and pharmaceuticals, SLNs may provide increased penetration of the compounds disclosed herein through the skin. SLNs may have ultraviolet (UV) resistant properties, occlusive properties which can be used to increase skin hydration, and good stability coalescence due to their solid nature, which reduces mobility and leakage of the active molecules.

Nanostructured lipid carriers (NLC) are a form of lipid nanoparticle formed by blending solid lipids with spatially incompatible liquid lipid compositions, forming an amorphous solid. NLCs may be of the imperfect type, amorphous type, or multiple type. NLC particles typically range in size from 10 nm to 1000 nm. When formulated from biodegradable and physiological lipids, NLCs show very little toxicity. Thus, NLC formulations may provide the benefits of reduced systemic side effects and higher drug loading capacity. NLCs may be designed to have a biphasic drug release pattern. For example, a first compound release profile may be immediate or controlled release, and a second compound release profile may be controlled or delayed release. Like SLNs, NLCs may also provide increased penetration of the compounds disclosed herein through the skin, ultraviolet (UV) resistant properties, occlusive properties which can be used to increase skin hydration, and good stability coalescence.

Nanoemulsions are kinetically and thermodynamically stable dispersions of liquid formed from an oil phase and a water phase in combination with a surfactant. The nanoemulsions disclosed herein may be oil in water, water in oil, or bicontinuous formulations. Properties of nanoemulsions may be designed by controlling method of preparation. Nanoemulsions are typically dispersed phase, comprising small particles or droplets having low oil or water interfacial tension. Nanoemulsions are tupically formed of a lipophilic core surrounded by a monomolecular layer of phospholipids. Nanoemulsions provide benefits, such as, low viscosity, high kinetic stability, high interfacial area, high solubilization capacity, and increased rate of absorption. In cosmetic and pharmaceuticals, nanoemulsions may provide rapid penetration and active transport of active ingredients and hydration to the skin. Nanoemulsions may be formulated into foams, creams, sprays, or liquids.

Certain nanoparticle formulations, such as SLNs, nanoemulsions, liposomes, and niosomes, may be used in moisturizing formulations, providing humectants that retain moisture for a prolonged period of time.

Metal nanoparticles may be designed to have specific properties and may be shaped as nanospheres, nanoshells, nanoclusters, nanorods, nanostars, nanocubes, branched, and nanotriangles. Shape, size, and dielectric properties of metal nanoparticles may have an effect on resonance frequency. Metal nanoparticles may be designed to have high drug loading capacity and effectively penetrate the cell wall by controlling size, surface area, and crystallinity. Benefits of metal nanoparticles include acceleration of blood circulation, anti-inflammatory properties, antiseptic properties, improvising firmness and elasticity of skin, delaying aging, and vitalizing skin metabolism. Exemplary metal nanoparticles are gold, silver, and copper. Such metal nanoparticles have been shown to provide strong antifungal and/or antimicrobial properties. Another exemplary metal nanoparticle is titanium dioxide (TiO₂), which has been shown to provide protection from ultraviolet (UV) radiation. Metal nanoparticles may be designed to be inert in nature, highly stable, biocompatible, and noncytotoxic. More than one metal may be used to form metal nanocomposites with selected properties.

Nanospheres are spherical nanoparticles having a core-shell structure. The compounds may be encapsulated, conjugated, dissolved, or otherwise entrapped in the nanoparticle. The nanospheres may be crystalline or amorphous structures. The nanospheres may be biodegradable or nonbiodegradable. Exemplary biodegradable biospheres include gelatin, modified starch, and albumin nanospheres. One exemplary nonbiodegradable nanosphere is polylactic acid. In cosmetics and pharmaceuticals, nanospheres may be used to deliver the compounds disclosed herein into a deep layer of the skin more precisely and efficiently. Nanospheres have been shown to provide protection against actinic aging.

Peptide-based nanomaterials are biomolecules made up of several amino acids linked by peptide bond. Peptides may generally provide rapid clearance in the kidney due to enzymatic degradation. Peptide nanomaterials may provide several benefits including targeting and accumulating capacity, small size, ease of production and customizability, and biocompatibility. Certain peptide nanomaterials may be designed to self-assemble into distinct shapes and sizes in response to environmental factors, such as temperature, pH, ionic strength, or molecular interaction between the host and peptide. Peptide nanoparticles may also be functionalized for targeted delivery. Functionalized peptides have been found to exhibit improved targetability and enhanced efficacy.

One exemplary peptide nanomaterial with mitochondrial-targeting ability is mitochondrial penetrating peptides (MPP). MPPs are cell penetrating peptides which can efficiently penetrate mitochondrial double membranes. MPPs are generally designed or selected to be positively charged peptides. Due to the strongly negative charge of mitochondrial membranes, positively charged peptides are capable of penetrating mitochondria. MPPs are described in more detail in “Mitochondrial targeted strategies and their application for cancer and other diseases treatment,” (Li, 2020), incorporated herein by reference in its entirety for all purposes. Thus, MPPs may be utilized as nanoparticle carriers and/or as surface modifications for targeted delivery. For instance, the compositions disclosed herein may be conjugated to an MPP or encapsulated in another carrier having an MPP surface functionalization for targeted delivery.

The formulations disclosed herein may comprise one or more skin penetration enhancer. Generally, small and moderately lipophilic molecules are likely to penetrate the skin barrier. Other compounds may require a suitable skin penetration enhancer to penetrate the barrier by either diminishing the barrier properties of the skin or actively driving movement of compounds across the skin with the input of external energy. Skin penetration enhancers are described in “Penetration Enhancement of Topical Formulations,” (Ng, 2018) and “Transdermal Delivery Systems in Cosmetics,” (Kim, 2020), each of which is incorporated herein by reference in its entirety for all purposes.

The nanoparticle delivery carriers disclosed herein may provide skin penetration enhancement for the compounds. The formulations disclosed herein may additionally or alternatively contain one or more chemical or physical skin penetration enhancers. Exemplary chemical skin penetration enhancers include alcohols, such as, ethanol and glycol, sulfoxides, such as, dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes and/or terpenoids. Exemplary physical skin penetration enhancers include rollers, scrapers, scrubbers, exfoliators, microdermabrasion needles, iontophoresis devices, electroporation devices, ultrasound devices, such as sonophoresis, thermal ablation, magnetophoresis, photomechanical waves, electron beam irradiation, and low light therapy devices, such as light emitting diode (LED) sources. Two or more skin penetration enhancers may be used in the formulation synergistically.

Chemical skin penetration enhancers may be included in dermatological, transdermal, cosmetic, and pharmaceutical products to enhance the dermal absorption of drug compounds. Chemical enhancers may enable or improve solubility, penetration, and/or absorption of the compounds disclosed herein. Exemplary chemical enhancers are described below.

Water (aqua) generally increases fluidity of the composition, providing higher permeability. Additionally, water hydrates the skin barrier, altering skin lipids and/or proteins for improved permeation. Hydrating compounds, such as glycerol and urea, may promote transdermal permeation by facilitating hydration of the stratum corneum and forming hydrophilic diffusion channels within the barrier.

Alcohol solvents, such as ethanol and propylene glycol, may provide penetration enhancement by increasing fluidity of the compound and also act as good solvents. Degree of permeation may be selected by controlling alkyl chain length of fatty alcohols.

Surfactants generally solubilize lipophilic agents, including active ingredients of the formulation as well as lipids within the stratum corneum. Thus, surfactants may enhance skin permeability by partitioning into the epithelial cell membranes and disrupting the packing of membrane lipids, forming structural defects that reduce membrane integrity. The effect of the surfactant on skin permeation may be designed by selecting concentration and type of surfactant. The surfactant may be anionic, cationic, or nonionic. Anionic surfactants may be selected for skin or hair applications because they interact with keratin and lipids. Cationic surfactants may be selected for skin applications because they interact with skin proteins via poler interactions. Non-ionic surfactants are generally less irritating to the skin and have better tolerability.

Fatty acids may increase percutaneous drug absorption. Long-chain fatty acids, which are carboxylic acids with typically long, unbranched aliphatic tails, have been demonstrated to increase percutaneous drug absorption as an effect of alkyl chain length. Low molecular weight alkanols may act as solubilizers to enhance the solubility of the compound in the fatty matric of the stratum corneum. Polyunsaturated fatty acids, such as linoleic, alpha-linoleic, and arachidonic acids, may enhance the skin permeation. One exemplary fatty acid chemical penetration enhancer is oleic acid. Oleic acid provides increased fluidity and reduced resistance toward the permeation of molecules.

Terpenes are hydrocarbons commonly found in plant extracts. Terpenoids are terpenes containing additional functional groups. One group of terpenes that may provide penetration enhancement are oxygen-containing terpenes. Exemplary oxygen containing terpenes include menthol, thymol, carvacrol, menthone, and cineole. Such terpenes may enhance penetration in a similar mechanism as alcohols. However, terpenes may be considered natural products. Benefits of terpenes include high percutaneous ability with minimal irritancy and toxicity.

The compounds of the disclosure may be formulated with a mitochondria-targeting agent. Exemplary mitochondrial targeting agents include antibodies, polymeric functional moieties, such as PEG, lipophilic cations, such as triphenylphosphine (TPP), and peptides such as mitochondrial targeting peptides (MPP).

The compounds of the disclosure may alternatively contain as pharmaceutically and/or cosmetically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically and/or cosmetically acceptable carriers can be used which include, for example, pharmaceutical grades of mannitol, lactose, starch, magnesium stearate, sodium saccharin, talcum, magnesium carbonate, and the like.

Compositions of the disclosure can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants.

In certain embodiments, compound(s) and compositions of the disclosure are administered in a time-release formulation, for example in a composition which includes a slow release polymer. Such compositions can be prepared with carriers that will protect against rapid release, for example a controlled release vehicle such as a polymer, microencapsulated delivery system or bioadhesive gel. Prolonged delivery, in various compositions of the invention can be brought about by including in the composition agents that delay absorption, for example, aluminum monosterate hydrogels and gelatin. When controlled release formulations is desired, controlled release binders suitable for use in accordance with the invention include any biocompatible controlled-release material which is inert to the active agent and which is capable of incorporating the biologically active agent. Numerous such materials are known in the art.

Formulations suitable for topical administration include solutions, oils, creams, emulsions, and gels containing, in addition to the active ingredient, such carriers as are known in the art. Topical formulations may be pharmaceutical or cosmetic formulations. In some embodiments, the compound is formulated as a shampoo, conditioner, spray, cream, gel, balm, body wash, soap, lotion, or make-up.

The compounds of the disclosure and compositions of the disclosure can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Suitable unit doses, i.e., effective amounts, may be determined during clinical trials designed appropriately for each of the conditions for which administration of a chosen compound is indicated and will, of course, vary depending on the desired clinical endpoint. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The compositions disclosed herein may be associated with a variety of natural products, and examples of such products are set out below. The compositions disclosed herein may be formulated as a natural product. The compositions disclosed herein may be administered in combination with a natural product. The compositions disclosed herein may be incorporated in a natural product. These natural products may be comprised of formulations or compositions disclosed throughout this disclosure.

Natural products may be or comprise products for commercial purposes, and may refer to dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, cosmetics, and spa treatments. A natural product referred to herein may comprise any one or more of the components described as a natural product to be incorporated into a composition or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

One skilled in the art will appreciate that suitable methods of administering a compound of the disclosure to a patient are available, and, although more than one route can be used to administer a particular compound, a particular route can provide a more immediate and more effective reaction than another route.

### EXAMPLES

The function and advantages of these and other embodiments can be better understood from the following examples. These examples are intended to be illustrative in nature and are not considered to be limiting the scope of the invention.

### Example 1: Identification of Compounds from Emblica Extract

To identify compounds present in emblica, an emblica extract was prepared and analyzed by HPLC.

In this example, 40.9 grams of caplets containing E. officinalis extract (Himalaya Drug Company, Sugar Land, TX Lot 112000924; each caplet contains 250 mg fruit extract (45% tannins) and 350 mg powder stem (2% tannins)) was stirred with 250 ml methanol for 6 hours at room temperature. The resulting solution was filtered to remove insoluble material and the methanol layer was stripped using a Rotovap (fraction 1). The solid material resulting from the filtration step was stirred with an additional 250 ml of methanol for 4 hours at room temperature, filtered through to remove insoluble material and a filter funnel and the methanol layer was stripped using a Rotovap (fraction 2). Fractions 1 and 2 were combined to obtain 8.25 grams of a dark, hydroscopic solid. 450 mg of solid isolated as described above was dissolved in 2 ml of methanol. The resulting solution was sonicated until all contents were in solution and subject to preparative HPLC purification using a ACCQPrep instrument (Teledyne ISCO, Lincoln, NE) with a Phenomenex Gemini® 5 μX-C18 110A 150×4.6 mm liquid chromatography column (Phenomenex, Torrance, CA). HPLC was carried out with a two solvent gradient as described in the table below.

| Gradient Time | | | Flow rate |
|---|---|---|---|
| (min) | % Solvent A | % Solvent B | (ml/min) |
| 0 | 0 | 100 | 42.5 |
| 5 | 0 | 100 | 42.5 |
| 20 | 90 | 10 | 45.2 |
| 24 | 90 | 10 | 42.5 |
| 26 | 5 | 95 | 42.5 |
| 28 | 5 | 95 | 42.5 |
| 29 | 50 | 50 | 42.5 |
| 43 | 50 | 50 | 42.5 |

Solvent A- acetonitrile;
Solvent B- water
Water purified through a PureLab ® Ultra water purification system (ELGA LabWater, Woodridge, IL).

The collected fractions were stripped and fractions transferred into eleven separate vials. 1 mg of each fraction was taken and dissolved in 1 ml of methanol in a 1.5 ml vial. The resulting solution was sonicated until all contents were in solution and subject to analytical HPLC analysis using an Agilent 1100 Series instrument (Agilent Technologies, Santa Clara, CA) with a Phenomenex Gemini® 5 μM NX-C18 110A 150×4.6 mm liquid chromatography column (Phenomenex, Torrance, CA). HPLC was carried out with a two solvent gradient as described in the table below.

| Gradient Time | | | Flow rate |
|---|---|---|---|
| (min) | % Solvent A | % Solvent B | (ml/min) |
| 0 | 0 | 100 | 1 |
| 5 | 0 | 100 | 1 |
| 20 | 97.5 | 2.5 | 1 |
| 24 | 90 | 10 | 1 |
| 26 | 5 | 95 | 1 |
| 28 | 5 | 95 | 1 |
| 29 | 100 | 0 | 1 |
| 43 | 100 | 0 | 1 |

Solvent A- acetonitrile with 0.1% trifluoroacetic acid (TFA)
Solvent B- water with 0.1% TFA
Water purified through a PureLab ® Ultra water purification system (ELGA LabWater, Woodridge, IL).

To identify specific compounds in the emblica extract the following standards were used:

| | | | Retention |
|---|---|---|---|
| Common name | Cas # | Source | Time (min) |
| Gallic Acid | 149-91-7 | Acros | 6.88 |
| | | Organics | 7.22 |
| Vanillic acid | 121-34-6 | Alfa Aesar | 27.64 |
| Chlorogenic acid | 327-97-9 | TCI America | 28.61 |
| Caffeic acid | 331-39-5 | TCI America | 28.65 |
| Syringic acid | 530-57-4 | Acros | 28.86 |
| | | Organics | |
| tp coumaric acid | 501-98-4 | TCI America | 29.52 |
| quercetin | 849061-97-8 | Acros | 30.63 |
| | | Organics | |
| Vitamin C | 50-81-7 | Sigma | 2.36 |
| | | Aldrich | 3.3 |

Standards were prepared by dissolving 1 mg of the standard in 1 ml methanol, sonicating the solution, and adding the solution to a 2 ml vial. Samples were subject to analytical HPLC as described above and to validate purity. A combined standard solution was created by adding 100 μl of each standard to a separate 2 ml vial. The standards were used to identify components in the emblica extract.

The following compounds were identified in the emblica extract prepared and analyzed as described above.

| | | Estimated amount |
|---|---|---|
| Common name | Cas # | in emblica tablets |
| Gallic Acid | 149-91-7 | 70% |
| Vanillic acid | 121-34-6 | Less than 2% |
| Chlorogenic acid | 327-97-9 | Less than 2% |
| Caffeic acid | 331-39-5 | Less than 2% |
| Syringic acid | 530-57-4 | Less than 2% |
| tp coumaric acid | 501-98-4 | Less than 2% |
| quercetin | 849061-97-8 | Less than 2% |
| Vitamin C | 50-81-7 | 20% |

In addition, approximately 20 other compounds were present but have not yet been identified.

### Example 2: Prophetic Example Showing in vivo Energy and Vitality Improving Effects of Application of Emblica Extract

Male and female subjects aged between 35 and 70 years showing aging in the form of visible eye wrinkles without further specific inclusion criteria will be evaluated for the study. The subjects will complete a baseline questionnaire of 10 closed questions with predefined options to be selected. The questionnaire will request information relating to baseline energy levels and vitality. Other baseline parameters will be measured for skin roughness (Ra, Rz) by DermaTOP (three-dimensional imaging of surface structure), skin hydration by Corneometer (Courage & Khazaka, Cologne, Germany) (electrical capacitance measurement), and skin elasticity by Cutometer (optical measurement of skin displacement during 300 mbar suction).

The subjects will apply emblica extract (2-5 drops of emblica extract oil) or placebo twice daily topically to one side of the face for approximately 12 weeks. The study will be performed in a split-face design. Anti-wrinkling properties will be measured periorbitally in the region of crowfeet. Skin moisturizing effects will be evaluated on the bones of the cheeks. Effect of the emblica extract will be compared to the reference product or placebo.

Subjects will complete a final questionnaire at completion of the study. The final questionnaire will request information relating to baseline energy levels and vitality. Skin roughness (Ra, Rz), skin hydration, and skin elasticity will also be measured at the completion of the study. Baseline results will be compared to final results.

It is expected that administration of emblica extract will improve energy levels and vitality. It is also expected that administration of emblica extract will improve aging-associated parameters including skin roughness, skin hydration, skin elasticity, and additional qualitative parameters measured by the questionnaire.

### Example 3: Restoration of Mitochondrial DNA Depletion and Function and Suppression of Inflammation by Emblica Extract In Vivo

In these experiments, the shaved dorsal skin of 8-9 weeks old female C57BL/6 control and mtDNA-depleter mice (expressing D1135A-POLG1) were treated topically with 200 μl of 50 mg/ml emblica extract ointment (prepared as described in the Methods section) or a corresponding amount of the control ointment (lacking emblica extract) daily beginning 1 week prior to the start of dox administration (200 mg/kg diet only) and continuing daily applications for 16 weeks (112 days).

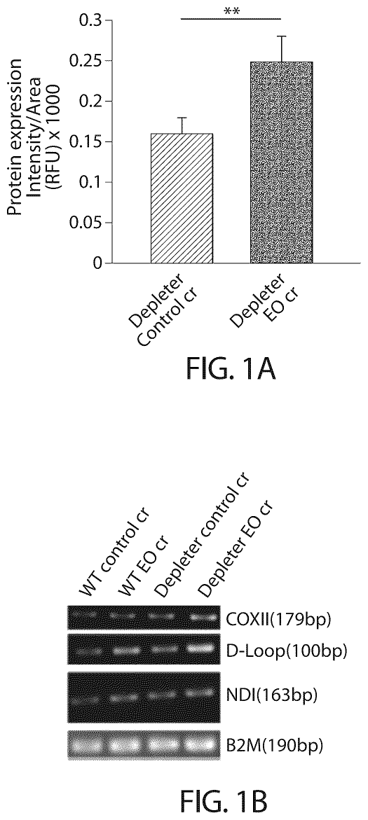

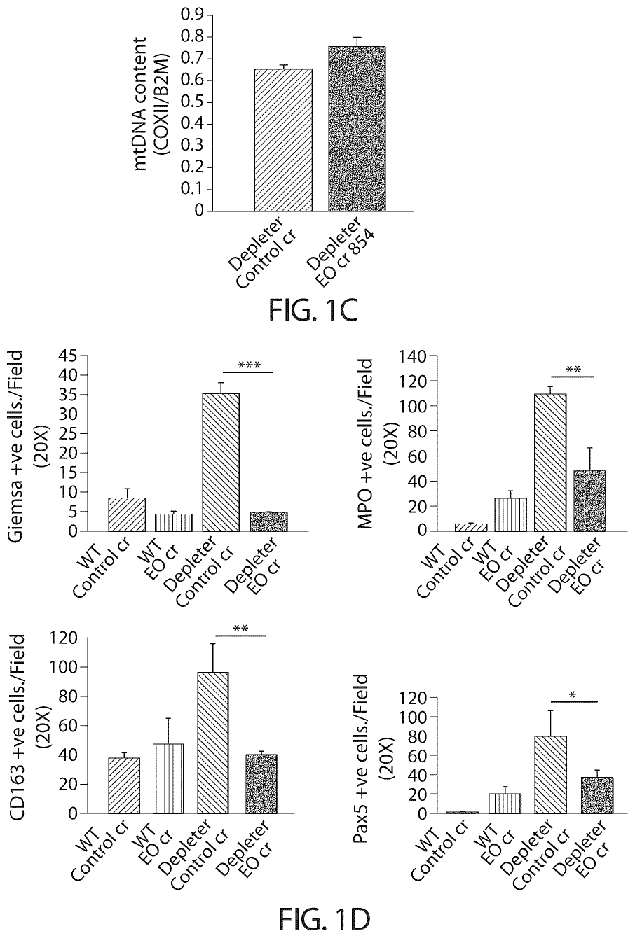

The effect of emblica extract treatment on reversal of mtDNA function was examined by staining paraffin embedded dorsal skin sections mtDNA-depleter mice treated with emblica extract ointment or control ointment with Oxphos complex IV antibody (COXII) (n=3). A statistically significant increase in COXII staining in mtDNA-depleter skin treated with emblica extract as compared to control treatment was observed (FIG. 1A). In addition, RT-PCR analysis of mtDNA encoded genes showed upregulation in mtDNA-depleter skin treated with emblica extract as compared to control treatment (FIG. 1B). Finally, an increase in mtDNA content was observed in skin samples from mtDNA-depleter mice treated with emblica extract as compared to control treatment (FIG. 1C).

As described herein, the skin of mtDNA depleter mice showed increase in dermal and peri-appendageal mixed inflammatory cells, including mast cells, neutrophils and lymphocytes. After treatment with emblica extract, the skin of mtDNA depleter mice showed a statistically significant decrease in dermal and peri-appendageal inflammatory cells (FIG. 1D) including mast cells (Giemsa+ve positive cells; p=3.56E-07), granulocytes (MPO+ve cells; P=0.002), macrophages and histiocytes (CD163+ve cells; p=0.007), and B lymphocytes (Pax-5+ve cells; p=0.042). Decreased expression of inflammatory genes in the skin samples of mtDNA-depleter mice treated with emblica extract as to mtDNA-depleter mice treated with control ointment was also observed (data not shown).

### Example 4: Reversal of Wrinkled Skin and Loss of Hair by Fucus Extract In Vivo

To investigate the ability of other agents to restore mitochondrial DA and function, a fucus extract was examined. In these experiments, the shaved dorsal skin of 8-9 weeks old female C57BL/6 control and mtDNA-depleter mice (expressing D1135A-POLG1) were treated topically with 200 μl of 50 mg/ml fucus extract ointment (prepared as described in the Methods section) or a corresponding amount of the control ointment (lacking fucus extract) daily beginning 1 week prior to the start of dox administration (200 mg/kg diet and 2 mg/mL in 5% sucrose water) and continued for 51 days (n=4 for each group).

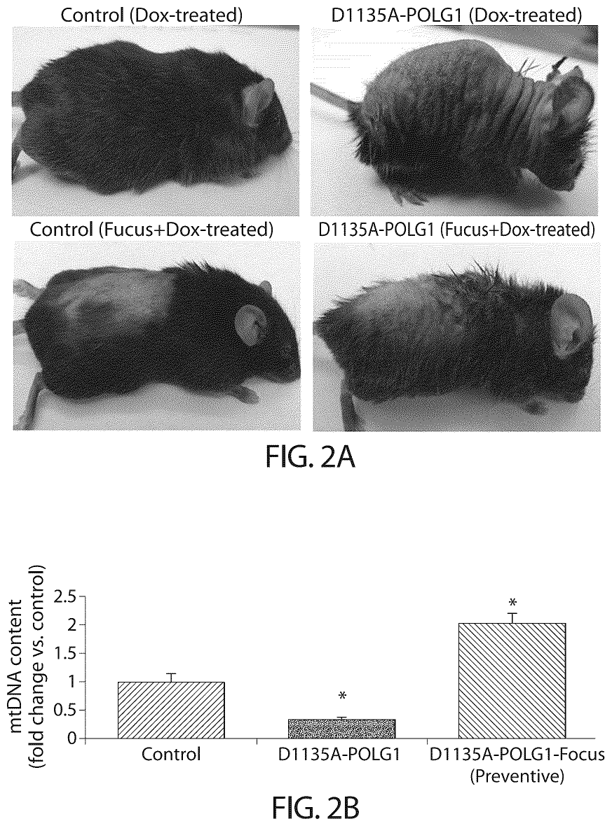

The results are shown in FIG. 2A. Consistent with previous results, dox administration to mtDNA-depleter mice resulted in significant hair loss and skin wrinkling phenotype. Administration of fucus extract partially reversed the hair loss and wrinkled skin phenotype as compared to mtDNA-depleter mice without fucus extract treatment (dox administration alone). Control mice showed no effects when treated with dox alone or dox in combination with fucus extract.

### Example 5: Restoration of Mitochondrial DNA Content by Fucus Extract In Vivo

The effect of fucus extract treatment on mitochondrial DNA content was also examined. Skin samples from the animals of Example 4 were collected and analyzed for mitochondrial DNA content and the results are shown in FIG. 2B. As shown in FIG. 2B, dox administration to mtDNA-depleter mice resulted in significant decrease in mitochondrial DNA content as compared to control mice. Administration of fucus extract fully restored mitochondrial DNA content. These results show that the beneficial effects of fucus extract on reversing the hair loss and wrinkled skin phenotype in mtDNA-depleter mice is correlated with preservation of mitochondrial DNA content.

### Example 6: Expression of Mitochondrial Biogenesis Regulatory Proteins In Vitro

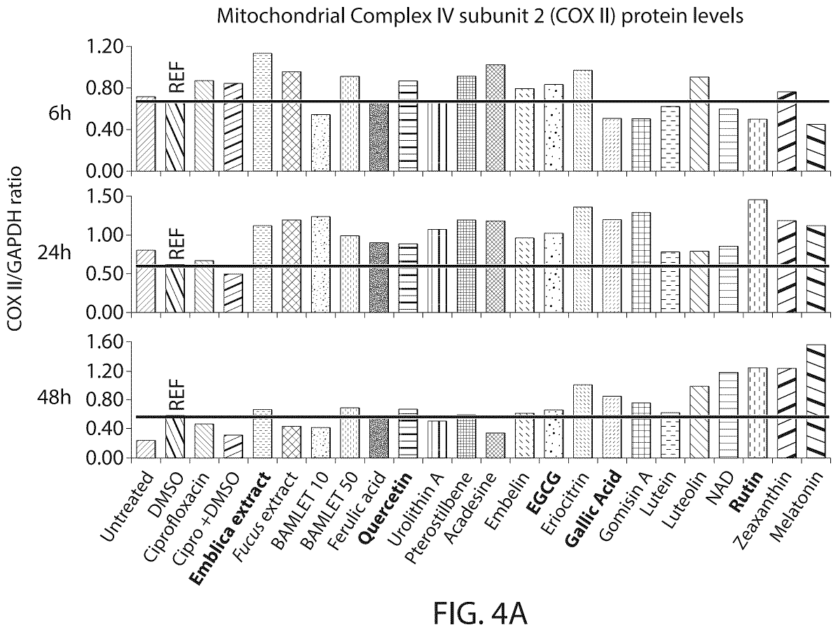

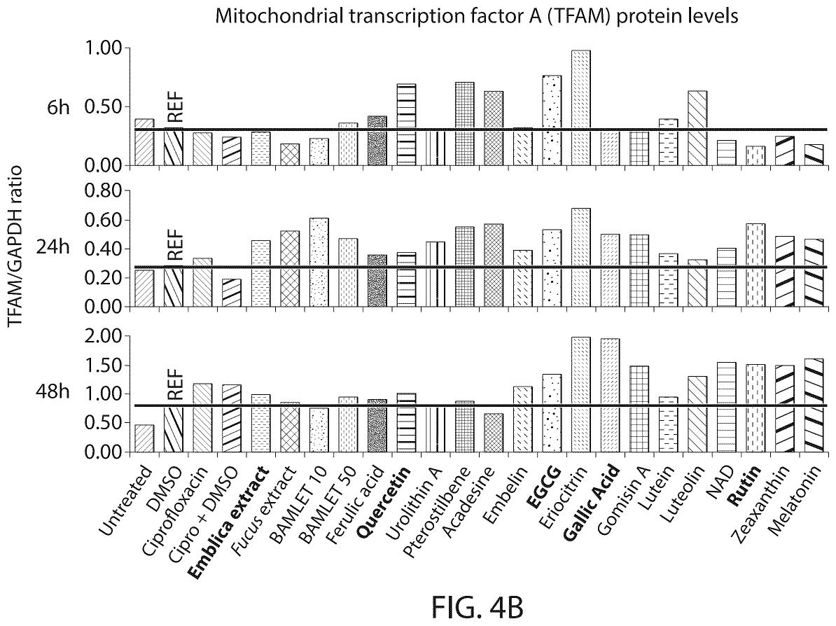

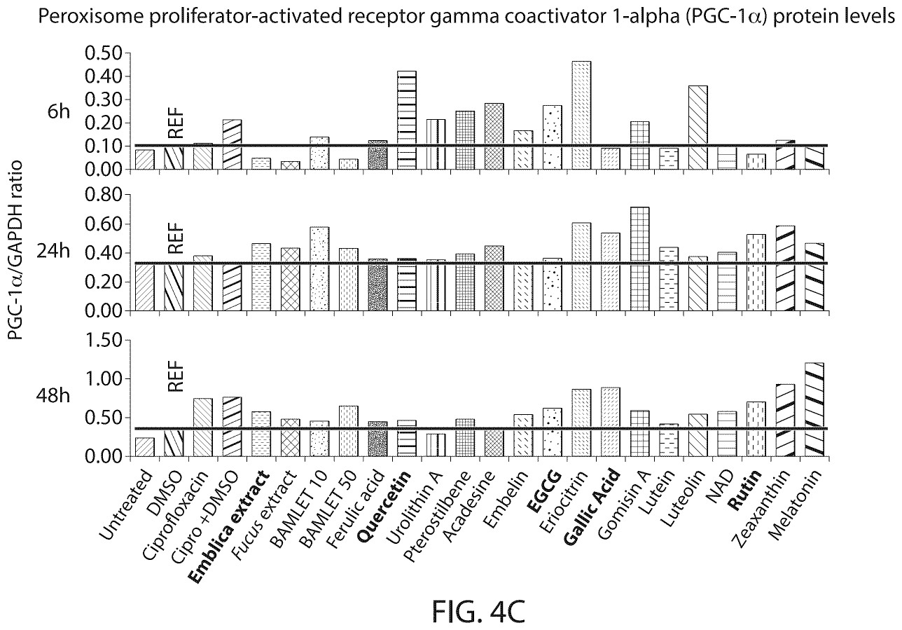

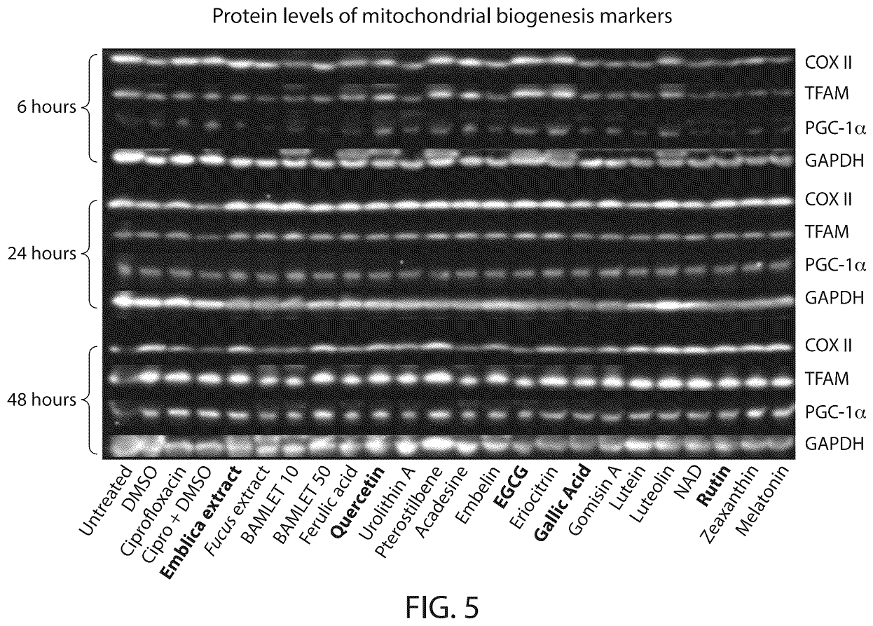

Emblica extract, components thereof, fucus extract, and related compounds were tested in vitro for induced expression of mitochondrial biogenesis regulatory proteins including mitochondrial complex IV subunit 2 (COXII), mitochondrial transcript factor A (TFAM), and peroxisome proliferator-activated receptor gamma coactivator 1-alpha (PGC-1a) at timepoints of 6 hours, 24 hours, and 48 hours post-administration. The results are presented in the graphs of FIGS. 4A-4C and the gel-electrophoresis image of FIG. 5.

Many of the tested compositions showed increased expression of the proteins as compared to a DMSO reference. Expression generally increased with increasing time.

### Example 7: Prophetic Example Showing Ex Vivo Modulation of Age Associated Read Out Parameters in Human Skin Organ Culture by Emblica Extract

Human full thickness skin organ cultures will receive 48×6 mm skin punches in 3 technical replicates. Emblica extract will be applied to the skin organ cultures in varying concentrations from 0.05% to 0.2% by systemic and topical application at 24 hours and 96 hours or 120 hours post-skin punching.

Skin cytotoxicity of the medium will be evaluated by LDH assay. Epidermal and dermal morphology will be evaluated in situ by Masson Trichome staining. Collagen I expression and fiber organization will be evaluated in situ by Collagen I immunofluorescence. MMP1 expression in the epidermis will be evaluated in situ by MMP1 immunofluorescence. Expression of mitochondrial markers, such as MTCO II and TFAM, will be evaluated in situ by correlation of mitochondrial energy and MTCO II and TFAM immunostaining. Mitochondrial mass will be evaluated in situ by VDAC immunostaining. Gene expression of MTCO II, TFAM, MMP1, VDAC, and Col1 will also be evaluated.

Remaining RNA, media, and tissue samples will be stored for follow-up analysis.

It is expected the emblica extract administration samples will show improved modulation of age associated parameters over vehicle control samples.

### Example 8: In Vivo Anti-Aging Effects on Human Skin by Application of Emblica Extract

Female subjects aged between 35 and 70 years showing visible eye wrinkles without further specific inclusion criteria were be evaluated for the study.

The subjects were instructed to apply emblica extract (2-5 drops of emblica extract oil) or placebo twice daily to one side of the face for approximately 12 weeks. The subjects were divided into a first group receiving a low dose of the composition and a second group receiving a high dose of the composition. The study was performed in a split-face design. Anti-wrinkling properties were be measured periorbitally in the region of crowfeet. Skin moisturizing effects were evaluated on the bones of the cheeks. Effect of the emblica extract is compared to the reference product or placebo. The subjects completed a questionnaire of 10 closed questions with predefined options to be selected after 6 weeks of the trial.

Subjects also completed a final questionnaire at completion of the study (after 12 weeks). Mid-study results were compared to final results.

The questionnaire prompted the study participants to answer the following question by selecting “agree” or “disagree.”
Q01 I have felt more energetic
Q02 My facial skin looks hydrated
Q03 I have received compliments on my skin from others
Q04 I have felt positive about my facial skin's appearance
Q05 I have been more productive at work and/or other activities
Q06 The lines on my facial skin seem less noticeable
Q07 My facial skin looks smoother
Q08 My facial skin looks more youthful
Q09 The firmness of my facial skin is improved
Q10 I feel the test oil is beneficial for me and I would like to continue using it after this trial

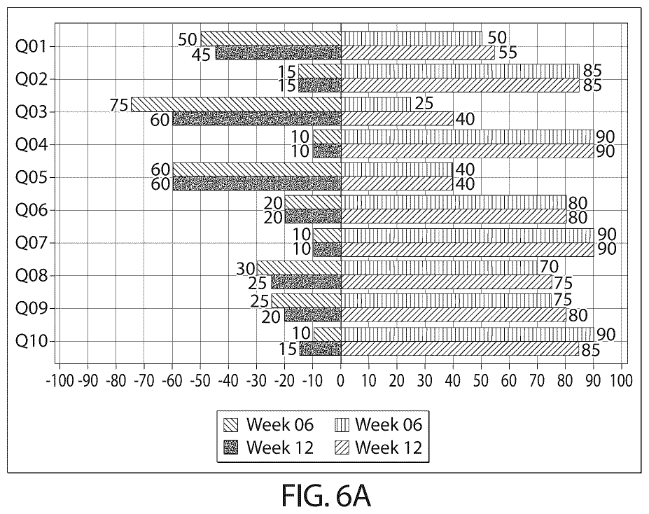

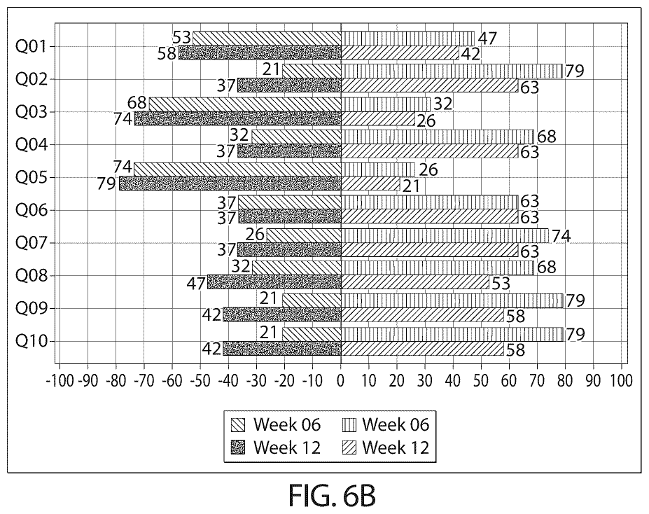

The results of the questionnaire are presented in the graphs of FIGS. 6A-6B. FIG. 6A includes results provided by the participants who received a low dose of the composition and FIG. 6B includes results provided by the participants who received a high dose of the composition.

### Example 9: Extended Expression of Mitochondrial Biogenesis Regulatory Proteins In Vitro

Emblica extract was tested in vitro for induced expression of mitochondrial biogenesis regulatory proteins including mitochondrial complex IV subunit 2 (COXII), mitochondrial transcript factor A (TFAM), and peroxisome proliferator-activated receptor gamma coactivator 1-alpha (PGC-1a) at timepoints of 6 hours, 12 hours, 24 hours, 48 hours, 72 hours, and 96 hours post-administration, generally as described in Example 6. Formulations of 0.01%, 0.05%, 0.1% and 0.2% emblica extract were tested. The results are presented in the graphs of FIGS. 7A-7F (COXII expression), 8A-8F (TFAM expression), and 9A-9F (PCG-1a expression). The general trend is that certain formulations induced greater expression after 96 hours than at earlier timepoints.

Emblica extract formulated with a nanoparticle delivery carrier was tested in vitro for induced expression of COXII and compared to a non-encapsulated composition at timepoints of 6 hours and 24 hours post-administration. The results are presented in the graphs of FIGS. 10A-10D. Specifically, FIG. 10A shows COXII expression at 6 hours after administration of a non-encapsulated composition. FIG. 10B shows COXII expression at 6 hours after administration of a nanoencapsulated composition. FIG. 10C shows COXII expression at 24 hours after administration of a non-encapsulated composition. FIG. 10D shows COXII expression at 24 hours after administration of a nanoencapsulated composition.

As shown in the data presented in FIGS. 10A-10D, all emblica extract formulations showed an increase in COXII expression and certain nanoencapsulated formulations showed a greater increase in COXII expression.

### Example 10: Ex Vivo Effects on Human Hair and Human Skin

Emblica extract was tested ex vivo on human hair and human skin samples.

### Human Hair Samples

Microscopic hair cycle staging at the anagen phase was measured at 6 days after administration (FIG. 11). As shown in the graph of FIG. 11, the 0.01% emblica extract formulation prolongs the anagen phase (active stage) of hair growth.

Hair matrix keratinocyte proliferation was measured at 6 days after administration (FIG. 12). As shown in the graph of FIG. 12, the 0.01% emblica extract formulation noticeably increases proliferation in the germinative hair matrix.

Immunohistochemical analysis was performed to analyze keratinocyte proliferation at 6 days after administration (FIG. 13). Melanin clumping was measured from the immunohistochemical analysis (FIG. 14). As shown in the graph of FIG. 14, the 0.01% emblica extract formulation does not cause hair follicle dystrophy, i.e., tissue fragility and melanin clumping.

### Human Skin Samples

LDH is a cytosolic enzyme present in many different cell types which is well established as a reliable indicator of cellular toxicity. LDH release was measured and normalized to the maximum release (FIG. 15). As shown in the graph of FIG. 15, the emblica extract formulation when administered at various concentrations (0.1%, 0.2%, and 1%) does not induce LDH release.

COL1A1 gene expression was measured at 24 hours and 5 days after administration (FIGS. 16A and 16B, respectively). The COL1A1 gene is responsible for collagen production.

As shown in the graphs of FIGS. 16A-16B, administration of the emblica extract increases COL1A1 gene expression after 24 hours and continues to increase COL1A1 gene expression after 5 days.

MTCO2, TFAM, and VDAC gene expression was measured at 24 hours after administration (FIGS. 17A-17C, respectively). MTCO2, TFAM, and VDAC are biomarkers of mitochondrial biogenesis and/or mitochondrial health. As shown in the graphs of FIGS. 17A-17B, the emblica extract induces MTCO2 and TFAM gene expression, respectively. As shown in the graph of FIG. 17C, various concentrations of the emblica extract (0.1%, 0.2%, and 1%) induce VDAC gene expression after 24 hours.

The expression of several target genes responsible for increasing mitochondrial health and activity, protecting mitochondria, and modulating mitochondrial respiration was validated using real time quantitative PCR. Briefly, after incubation, the treated ex vivo cells were lysed with Direct PCR Lysis Reagent (Viagen Biotech). DNA was extracted using TaqMan Fast Advanced Master Mix (Life Technologies) according to the manufacturer's instructions. TaqMan Gene Expression Assay (Applied Biosystems) was performed with each appropriate primer according to a standard real time quantitative PCR protocol. The data were analyzed according to the 2—ΔΔCT method.

The target genes include basic fibroblast growth factor (FGF2), fibroblast growth factor receptor 1 (FGFR1), cytochrome C oxidase subunit 7A1 (COX7A1), pyruvate dehydrogenase kinase 4 (PDK4), adenine nucleotide translocase lysine methyltransferase (FAM173A), mitochondrial ribosomal protein L12 (MRPL12), and wingless-type MMTV integration site family, member 11 (WNT11). The expression was measured at 24 hours or 120 hours after administration. The results are shown in the graphs of FIGS. 18A-18B. As shown in the graphs of FIGS. 18A-18B, when applied topically, the emblica extract formulation activates all u7 critical genes at the tested timepoint.

### Example 11: Expression of Mitochondrial Biogenesis Regulatory Proteins In Vitro

Selected constituents of emblica extract were tested in vitro for induced expression of mitochondrial biogenesis regulatory proteins including COXII and TFAM at timepoints of 6, 12, 24, and 48 hours post-administration, generally as described in Example 6. Formulations of varying concentrations were tested. The results are presented in the graphs of FIGS. 19A through 31D. Briefly, chebulagic acid, chebulinic acid, kaempferol, ellagic acid, ascorbic acid, citric acid, gallic acid, quercetin, punicalagin were tested at concentrations ranging from 2.5 μM to 100 μM for induced expression of the target proteins.

### Example 12: Nanoencapsulation of Emblica Extract and Chebulinic Acid

Emblica extract and chebulinic acid formulations and nanoencapsulated chebulinic acid formulations were tested in vitro for induced expression of mitochondrial biogenesis regulatory proteins COXII and TFAM at timepoints of 6, 12, 18, and 24 hours post-administration, generally as described in Example 6. Formulations of varying concentrations were tested. The results are presented in the graphs of FIGS. 32A-32D (COXII) and 33A-33D (TFAM).

Induced expression of COXII at 6 hours post-administration of varying concentrations of nanoparticle loading from 0.01% to 0.2% emblica extract and 50 to 400 μg/L was tested. The results are presented in the graphs of FIGS. 34A-34B. As shown in the graphs of FIGS. 34A-34B, the greater effect was observed as a result of greater compound loading.

Induced expression of COXII and TFAM at 6, 12, 24, and 48 hours post-administration of nanoparticle compositions sized from 4.4 μm to 95 μm (with and without emblica extract) was tested. The results are presented in the graphs of FIGS. 35A-35D (COXII), FIGS. 36A-36D (TFAM), and FIGS. 37A-37D (COXII). In the graphs, the x axis legend is as follows:
Nano A: Y100 (emblica extract) powder loaded (40% LF; mean particle size=11.6 μm)
Nano B: Y100 (emblica extract) oil loaded (42% LF; mean particle size=9.4 μm)
Nano C: undoped blank (mean particle size=7.7 μm)
Nano D: large size octyl-doped blank (mean particle size=95 μm)
Nano E; small size octyl-doped blank (mean particle size=4.4 μm)

As shown in the graphs of FIGS. 35A-35D, 36A-36D, and 37A-37D, the greater effect was generally observed as a result of smaller particles.

Induced expression of COXII and TFAM at 6, 12, 24, and 48 hours post-administration of nanoparticle compositions having a similar size (with and without emblica extract) (with octyl-doping and without octyl-doping) was tested. The results are presented in the graphs of FIGS. 38A-38D (COXII), and FIGS. 39A-39D (TFAM). As shown in the data presented in FIGS. 38A-38D and 39A-39D, octyl-doped particles showed a greater expression than their undoped counterparts.

Overall, the data show that induction of mitochondrial biogenesis depends on nanoparticle load and the size of nanoparticles affects mitochondrial biogenesis. Differences in nanoparticle preparation (between octyl-doped and undoped) and load have an effect on mitochondrial biogenesis induction.

### Example 13: Methods of Screening Emblicannins with a Deep Neural Network

A Deep Neural Network based on the Transformer architecture, specifically the Electra design, was developed for the purpose of screening compounds. Using a large and extensively augmented dataset of chemical reactions, the base neural network was trained to model a wide range of general chemical reactions with a nonexclusive focus on Organic Chemistry. Data Augmentation was used to ensure that the neural network learns the statistical patterns of general chemistry irrespective of the sequence order of the base training data. Thus, the neural network is forced to learn how chemical reactions work without regard to arbitrary changes in the sequence of SMILES representations or the order in which reactant molecules are presented.

In this example, the neural network is useful in distilling all relevant information about a chemical or a chemical reaction into a compact representation known as an ‘embedding’. In the case of the neural network, the embedding is a sequence of 768 numbers which comprise all relevant information that the model can determine about a chemical or a chemical reaction. This is in effect a 768-dimensional vector that can be used to determine the similarity of any two compounds, among other uses. Machine Learning techniques such as Partial Component Analysis (PCA) and T-distributed Stochastic Neighbor Embedding (T-SNE)—techniques generally referred to as “dimensionality-reduction”—together with mathematical measures such as Cosine Similarity were used on the 768-dimensional embeddings to detect compounds which are chemically similar to candidate molecules identified by human experts. This made it possible to winnow the field of candidate Natural Product molecules down by many orders of magnitude by selecting the most chemically similar compounds available for testing.

In addition to the “unsupervised learning” techniques described above, the model has further uses. For example, labeled training datasets using methods described in “NPClassifier: A Deep Neural Network-Based Structural Classification Tool for Natural Products,” (Kim, 2021) (incorporated herein by reference in its entirety for all purposes), make it possible to fine-tune the network on specific tasks such as the classification of natural compounds into major and minor categories such as alkaloids/amino acids/peptides/carbohydrates and macrolides/linear polyketides/tetramate alkaloids etc., limited only by the extent of such labeled secondary datasets. By training the network on this secondary task through the technique of Transfer Learning, the network becomes more capable of identifying useful compounds by both category and chemical similarity.

### Materials and Methods

Creation of mtDNA-Depleter Mice

D1135A-POLG1 site-directed mutation was created in the full-length human POLG1 complementary DNA (cDNA) using the site-directed mutagenesis kit (Agilent, Santa Clara, CA, USA). The primer sequences used for site-directed mutagenesis are as follows, with the mutated site in upper case: D1135A F:5′-gcatcagcatccatgCGgaggttcgctacctgg-3′ and D1135A R:5′-ccaggtagcgaacctcCGcatggatgctgatgc-3′. Mutations were confirmed by sequencing. D1135A-POLG1 cDNA was subcloned into the dox-inducible mammalian expression vector, pTRE-Tight-BI-AcGFP1 (Clontech, Palo Alto, CA, USA). To obtain germline transmission of human D1135A-POLG1 (POLG1-DN), microinjection of the pTRE-Tight-BI-AcGFP1-D1135A-POLG1 construct into fertilized oocytes from C57BL/6 mouse was carried out. Potential founders were identified by screening genomic DNA from tail biopsies for the presence of the human Polg1 transgene using the PCR. The heterozygous human POLG1-positive (+/POLG1-DN⁺) founder male mice were mated with CAG-rtTA3 (rtTA) C57BL/6 female mice (Jackson Laboratories, stock no. 016532) to obtain +/POLG1-DN⁺ rtTA heterozygous transgenic mice. The +/POLG1-DN⁺ rtTA heterozygous mice were intercrossed to generate homozygous POLG1-DN⁺ rtTA⁺/POLG1-DN⁺ rtTA mice (mtDNA-depleter mice). This cross resulted in normal litter size (6-7 pups) and Mendelian distributions of genotypes, that is, 1:2:1 distribution of wild-type, heterozygous +/POLG1-DN⁺ or +/rtTA⁺ and homozygous POLG1-DN⁺ rtTA⁺/POLG1-DN⁺ rtTA showing that homozygosity for POLG1-DN allele does not result in embryonic or postnatal lethality. All the mice were given dox in diet (200 mg/kg diet) and water (2 mg/ml dox in 5% sucrose water) ad libitum. All animal experiments were conducted by following guidelines established by the Institutional Animal Care and Use Committee.

### Histological and Immunohistochemical Analyses

Skin from the dorsal side as well as other tissues was fixed in buffered formalin, embedded in paraffin, sectioned (5 μM), and stained with hematoxylin and eosin. Skin sections were stained with Giemsa stain to detect mast cells, while MPO, CD3, CD163, and Pax-5 antibodies were used for detection of other types of inflammatory cells by immunohistochemical analyses (Carson, et al., Histotechnology: A Self-Instruction Text, 3 ed., American Society for Clinical Pathology Press, Hong Kong, 2009).

RT-PCR and mtDNA Content Analyses

To measure relative gene expression by RT-PCR, total cellular RNA from the skin samples was isolated using Trizol (Invitrogen, Carlsbad, CA, USA). Approximately, 1000-2000 ng RNA was normalized across samples, and cDNA was generated using the Iscript cDNA synthesis kit (Bio-Rad Laboratories, Hercules, CA, USA). cDNA was then subjected to RT-PCR using Green Taq PCR mixture (Promega, Madison, WI, USA) and gene-specific primers as given in Table 1 below. PCR products were run on 1.5 to 2% agarose gel and photographed using gel documentation system. At least three biological replicates were used in each PCR. β2-Microglobulin or RNU6B was used as an internal control in each PCR.

mtDNA content analyses in the skin and other tissues were carried out as reported earlier (Singh et al., PloS One, 10, e0139846, 2015). Briefly, the mtDNA content was analyzed by real-time PCR by absolute quantification with the following primers: mMitoF: 5′-CTAGAAACCCCGAAACCAAA-3′, mMitoR: 5′-CCAGCTATCACCAAGCTCGT-3′, mB2MF: 5′-ATGGGAAGCCGAACATACTG-3′, and mB2MR: 5′-CAGTCTCAGTGGGGGTGAAT-3′. Beta-2-Microglobulin (B2M) was used as an internal control.

TABLE 7

| | | |
|---|---|---|
| Target | Forward primer | Reverse primer |

Primers used for genotyping

| | | |
|---|---|---|
| POLG1 | CAA GGT CCA | CTC TGT ACC |
| | GAG AGA AAC | ACC CAA TTC |
| | TG | AC |
| CAG-rtTA | CTG CTG TCC | CGA AAC TCT |
| | ATT CCT TAT | GGT TGA CAT |
| | TC | G |
| GFP | GGG CAA TAA | TGG ACA GGT |
| | GAT GGA GTA | AGT GGT TAT |
| | CA | CG |

Primers used for RT-PCR

| | | |
|---|---|---|
| POLG1 | CCA GGG AGA | CAA ATT CCT |
| | GTT TAT AAC | CAA ACA GCC |
| | CA | AC |
| COXII | GGC ACC TTC | CGG TTG TTG |
| | ACC AAA ATC | ATT AGG CGT |
| | AC | TT |
| NDI | CCT ATC ACC | TTG CTG CTT |
| | CTT GCC ATC | CAG TTG ATC |
| | AT | GT |
| NF-κB | TGG CCG TGG | GCA TCA CCC |
| | AGT ACG ACA | TCC AGA AGC |
| | A | A |
| MMP2 | ACC TGA ACA | CTT CCG CAT |
| | CTT TCT ATG | GGT CTC GAT |
| | GCT | G |
| | G | |
| MMP9 | CTG GAC AGC | CTC GCG GCA |
| | CAG ACA CTA | AGT CTT CAG |
| | AAG | AG |
| TIMP1 | CTT GGT TCC | ACC TGA TCC |
| | CTG GCG TAC | GTC CAC AAA |
| | TC | CAG |
| COL1A1 | CTG GCG GTT | TTC CAG GCA |
| | CAG GTC CAA | ATC CAC GAG |
| | T | C |
| Cyclooxygenase 2 | AAC CGC ATT | CAT GTT CCA |
| | GCC TCT GAA | GGA GGA TGG |
| | T | AG |
| CCL5 | AGA TCT CTG | GGA GCA CTT |
| | CAG CTG CCC | GCT GCT GGT |
| | TCA | GTA G |
| IL28a | AGG TCT GGG | CTG TGG CCT |
| | AGA ACA TGA | GAA GCT GTG |
| | CTG | TA |
| IFNB1 | GTC ATG GGT | CAG ACC CCT |
| | TTC TCA TGA | TCC AGT GAT |
| | AGA ACA G | TCA TC |
| VEGF | GAG GAT GTC | GTC GTG TTT |
| | CTC ACT CGG | CTG GAA GTG |
| | ATG | AGC AA |
| IGF1R | CGA GCT TCC | CAC GTT ATG |
| | TGT GAA AGT | ATG ATT CGG |
| | GAT GT | TTC TTC |
| | GGA CAT TTC | AGA GAG AGT |
| Klotho | CCT GTG ACT | AGT GTC CAC |
| | TTG C | TTG AAC GT |
| MRPS5 | AAC CAC TGT | AGT CTC TGC |
| | CTG ACC AGC | TAA TGC GCC |
| | TTG | TTT |
| RNU6B | CTC GCT TCG | AAC GCT TCA |
| | GCA GCA CA | CGA ATT TGC |
| | | GT |
| B2M | ATG GGA AGC | CAG TCT CAG |
| | CGA ACA TAC | TGG GGG TGA |
| | TG | AT |

### Microarray Gene Analysis

Total RNA samples were extracted from POLG1 D1135A expressing MCF-7 cells after 5 days of dox induction and from the cells grown in the absence of dox for 5 days by Trizol extraction method (Invitrogen). Illumina human microarray gene expression analysis was performed with total RNA samples as described earlier.

### BN-PAGE and Western Blot Analyses

Mitochondrial isolation was carried out as previously described (Johnstone et al., J Biol Chem, 277, 42197-42204, 2002). To analyze mitochondrial OXPHOS super complexes, Blue-Native polyacrylamide gel electrophoresis (BN-PAGE) was performed with mitochondrial fractions prepared from the skin samples as described previously (Schagger et al., Mehtods Enzymol, 260, 190-202, 1995). Protein expression of mitochondrial OXPHOS subunits in the skin samples was carried out following standard immunoblots. A premixed cocktail containing primary monoclonal antibodies (Mitosciences, Eugene, OR, USA) against subunits of OXPHOS complexes was used to detect OXPHOS super complexes in BN-PAGE analyses and protein expression of OXPHOS subunits in immunoblot analyses. Voltage-dependent anion channel (VDAC) or β-actin antibodies were used as loading controls.

### Analysis of Enzymatic Activities of OXPHOS Complexes

Isolated mitochondria were used for the measurement of enzymatic activities of OXPHOS complexes as previously described (Owens et al., PloS One, 6, e23846, 2011).

### Transmission Electron Microscopy

Transmission electron microscopic analyses of skin samples were carried as described previously (NAG et al., J Mol Cell Cardiol, 15, 301-317, 1983). Images were taken using the FEI-Tecnai electron microscope.

### Cell Culture

Skin fibroblasts from wild-type C57BL/6 (control cells) and mtDNA-depleter mice containing the D1135A-POLG1 site-directed mutation (POLG1-DN cells) were generated and spontaneously immortalized as described (Todaro et al., J Cell Biol, 17, 299-313 (1963). These cells were maintained in DMEM/F12 (Cellgro, Herndon, VA) supplemented with 10% FBS (Atlanta Biologicals, Lawrenceville, GA). To induce POLG1-DN expression in skin fibroblasts, 1 μg/ml dox dissolved in water was added to the cells in culture and after 6 days of incubation, cells were washed with PBS and collected in Trizol for isolation of total RNA.

To estimate cell proliferation and cell survival, MTT assays were carried out as described previously (Ronghe et al., J Steroid Biochem Mol Biol, 144 PtB, 500-512, 2014). Both control and POLG1-DN cells were first treated with dox (1 μg/ml) for 3 days and then cells were plated at a density of 3000 cells/well in 96 well plate with or without dox (1 μg/ml) containing culture media. Readings were taken at every 24 hours.

### Preparation of Emblica Extract

Emblica officinalis was obtained from commercially available caplets (Himalaya Drug Company, Sugar Land, TX). Each caplet contains 600 mg (250 mg fruit extract (45% tannins), 350 mg powder stem (2% tannins). Caplets were ground and dissolved in sterile water to make 100 mg/ml solution and then filtered. The emblica solution was then mixed with an appropriate ointment base for topical application. Suitable ointment bases include, but are not limited to, dermabase ointment (MARCELLE®; water, mineral oil, propylene glycol, stearyl alcohol, cetyl esters, cetyl alcohol, glyceryl stearate, sodium lauryl sulfate, lecithin, and methylparaben) and 1:1 w/v) and Geritrex hydrophilic ointment (NDC 54162-670-14). The ointment base and emblica solution may be added at any convenient ratio, for example from 1:5 w/v emblica solution to ointment base to 5:1 w/v emblica solution to ointment base. The ointment base and emblica solution may be added at a 1:1 w/v ratio emblica solution to ointment base.

### Preparation of Fucus Extract

Fucus vesiculosus (also known as bladder wrack, black tang, rockweed, bladder fucus, sea oak, cut weed, dyers fucus, red fucus, and rock wrack) powder was obtained from Maine Coast Sea Vegetables, Inc. (Hancock, MN). An aqueous solution (100 mg/ml) solution was prepared from the fucus powder and filtered. The fucus solution was then mixed with an appropriate ointment base for topical application. Suitable ointment bases include, but are not limited to, dermabase ointment (MARCELLE®; water, mineral oil, propylene glycol, stearyl alcohol, cetyl esters, cetyl alcohol, glyceryl stearate, sodium lauryl sulfate, lecithin, and methylparaben) and 1:1 w/v) and Geritrex hydrophilic ointment (NDC 54162-670-14). The ointment base and fucus solution may be added at any convenient ratio, for example from 1:5 w/v fucus solution to ointment base to 5:1 w/v fucus solution to ointment base. The ointment base and fucus solution may be added at a 1:1 w/v ratio fucus solution to ointment base.

### Emblica Extract In Vivo Experimental Design

For animal experiments, the dorsal skin of the mice was depilated (for example, shaved under low-dose isoflurane inhalation anesthesia) approximately 2 days before initiation of administration of a composition to the skin. Various active compositions and control compositions were applied topically to the dorsal skin daily as described. The skin of the mice was depilated prior to collection and analysis.

Emblica extract ointment at a concentration of 100 mg/mL aqueous solution was applied topically each day to a defined shaved area of the dorsal skin of the mice. Control compositions comprised the same amount of ointment base without addition of emblica extract.

Fucus extract ointment at a concentration of 100 mg/mL aqueous solution was applied topically each day to a defined shaved area of the dorsal skin of the mice. Control compositions comprised the same amount of ointment base without addition of fucus extract.

mtDNA-depleter mice (containing the D1135A-POLG1 mutation) as well as wild-type C57BL/6 mice (control) were used in the prevention and therapeutic experiments.

In the preventive experiments, daily emblica extract treatment as described above was started 7 days before starting the dox-mediated induction of POLG1-DN. Both mtDNA-depleter and wild-type C57BL/6 mice were divided in two treatment groups: i) a control group (treated with ointment base only, n=5); and ii) a test group (treated with emblica extract ointment, n=5). Daily administration continued through the end of the experiment (up to 112 days).

In the therapeutic experiments, the wild-type C57BL/6 mice (control) and mtDNA-depleter mice were first induced with dox for 30 days and then emblica extract treatment as described above was applied daily to the end of the experiment (up to 112 days) Both mtDNA-depleter and wild-type C57BL/6 mice were divided in two treatment groups: i) a control group (treated with ointment base only, n=5); and ii) a test group (treated with emblica extract ointment, n=5).

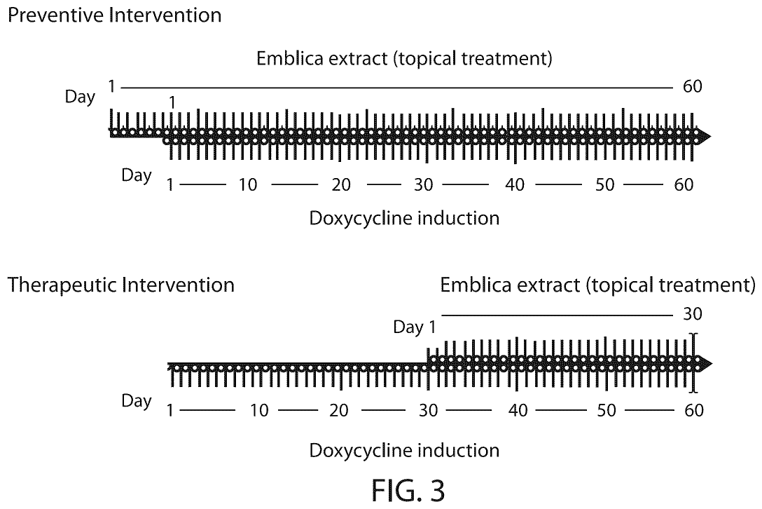

For periods of dox administration, mice were given dox in diet (200 mg/kg diet) and water (2 mg/ml dox in 5% sucrose water) ad libitum for Examples 1 to 13 and mice were given dox in diet (2 mg/kg diet) and water (2 mg/ml dox in 5% sucrose water) ad libitum for Examples 14 to 15. Schematics of both preventive and therapeutic in vivo experiments are shown in FIG. 3, noting that in certain experiments in Examples 3-5 the time course of emblica extract or fucus extract administration continued beyond the end of the time periods specified in FIG. 3 (i.e., up to 112 days).

### Statistical Analyses

Statistical analyses were performed using unpaired Student's t test. Data are expressed as mean±s.e.m. P values <0.05 were considered significant. All cellular experiments were repeated at least three times.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1.-62. (canceled)

63. A preparation comprising a treatment agent selected from an isolated compound constituent of emblica extract, chebula extract, or a metabolite thereof, a compound having a similarity score of at least 95% with the isolated compound constituent of emblica extract, chebula extract, or metabolite thereof, a pharmaceutically acceptable form thereof, or a combination thereof in a pharmaceutically or cosmetically acceptable vehicle.

64. The preparation of claim 63, wherein the treatment agent comprises an isolated ellagitannin.

65. The preparation of claim 63, wherein the treatment agent is isolated emblicanin A, isolated emblicanin B, isolated chebulinic acid, isolated chebulagic acid, isolated gallic acid, isolated kaempferol, isolated punicalagin, or a combination thereof.

66. The preparation of claim 63, wherein the treatment agent is derived from, purified from, or isolated from an extract.

67. The preparation of claim 66, wherein the extract is an extract of Emblica officinalis, Terminilia chebula, Terminalia arborea, or Lumnitzera racemose.

68. The preparation of claim 63, wherein the treatment agent is synthetic.

69. The preparation of claim 63, wherein the treatment agent is formulated in a nanoparticle-based delivery carrier.

70. The preparation of claim 69, wherein the nanoparticle-based delivery carrier has an average size of about 10 to 5000 nm.

71. The preparation of claim 69, wherein the nanoparticle-based delivery carrier comprises and/or is functionalized with a carbon-based nanomaterial, liposomal delivery vehicle, polymeric nanocarrier, micelle, dendrimer, lipophilic cation, solid-lipid nanoparticle (SLN), peptide-based nanomaterial, nanostructured lipid carrier (NLC), niosome, nanoemulsion, metal nanoparticle, nanosphere, polymerosome, cubosome, or a combination thereof.

72. The preparation of claim 69, wherein the nanoparticle-based delivery carrier is functionalized with a mitochondrial targeting agent.

73. The preparation of claim 72, wherein the mitochondrial targeting agent is selected from a mitochondrial targeting antibody, a polymeric functional moiety (e.g., polyethylene glycol (PEG)), a lipophilic cation (e.g., triphenylphosphine (TPP)), a mitochondrial targeting peptide (MPP), a derivative thereof, or a combination thereof.

74. The preparation of claim 69, wherein the treatment agent is conjugated to a mitochondrial targeting agent.

75. The preparation of claim 74, wherein the mitochondrial targeting agent is selected from a mitochondrial targeting antibody, a polymeric functional moiety (e.g., polyethylene glycol (PEG)), a lipophilic cation (e.g., triphenylphosphine (TPP)), a mitochondrial targeting peptide (MPP), a derivative thereof, or a combination thereof.

76. The preparation of claim 63, formulated for topical administration to the subject.

77. The preparation of claim 76, formulated as a topical solution, oil, cream, emulsion, foam, or gel.

78. The preparation of claim 77, formulated as a shampoo, conditioner, spray, cream, foam, gel, balm, body wash, soap, lotion, or make-up.

79. The preparation of claim 63, formulated for parenteral administration to the subject.

80. The preparation of claim 79, formulated as a parenteral liquid solution.

81. The preparation of claim 63, formulated for enteral administration to the subject.

82. The preparation of claim 81, formulated as an enteral capsule or tablet, or dietary supplement or food, e.g., food, food supplement, medical food, food additive, nutraceutical, or drink.

83. The preparation of claim 63, wherein the treatment agent is purified, e.g., at least 80% purified, at least 85% purified, at least 90% purified, at least 95% purified, at least 98% purified, at least 99% purified, at least 99.9% purified, at least 99.99% purified, or at least 99.999% purified.

84. The preparation of claim 63, formulated for immediate release.

85. The preparation of claim 63, formulated for extended release, e.g., controlled or sustained release.

86. The preparation of claim 63, comprising a skin penetration enhancer, e.g., a chemical skin penetration enhancer or a physical skin penetration enhancer.

87. The preparation of claim 86, wherein the chemical skin penetration enhancer comprises niosomes, proniosomes, liposomes, phospholipids, glycerin, alcohols, e.g., ethanol or glycol, sulfoxides, e.g., dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes or terpenoids, or a combination thereof.

88. The preparation of claim 86, wherein the physical skin penetration enhancer comprises a roller, scraper, scrubber, exfoliator, microdermabrasion needles, iontophoresis device, electroporation device, ultrasound device, e.g., sonophoresis device, thermal ablation, magnetophoresis, photomechanical waves, electron beam irradiation, low light therapy device, e.g., a light emitting diode (LED) source, or a combination thereof.

89. The preparation of claim 63, wherein the treatment agent is formulated for administration in combination with caffeine, a B vitamin, e.g., B1, B2, B3, B5, B6, B8, B9 and/or B12 vitamin, vitamin C, iron, magnesium, zinc, a UV-blocking agent, moisturizer, sunscreen, wrinkle cream, retinoid, alpha-hydroxy acid, beta-hydroxy acid, squalene, antioxidant, e.g., CoQ10, vitamin E, carotenoid, e.g., beta-carotene, mineral, e.g., selenium or manganese, glutathione, lipoic acid, flavonoid, betaflavonoid, phenol, polyphenol, phytoestrogen, mitoquinol mesylate, ubiquinone, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, soy, dermal filler or injection, e.g. hyaluronic acid or calcium hydroxylapatite, botulinum toxin, laser resurfacing procedure, ultrasound therapy, chemical peel, e.g., glycolic acid peel, trichloroacetic acid or salicylic acid, dermabrasion procedure, or combination thereof.

90. The preparation of claim 89, wherein the treatment agent is formulated for administration in combination with a second agent approved to treat or commonly used to treat a target disease or condition or a symptom thereof.

91. The preparation of claim 90, wherein the target disease or condition or symptom thereof is associated with mitochondrial dysfunction, an inflammatory response, or inflammation.

92. The preparation of claim 63, formulated to have a concentration of 0.01% to 2% of the compound.

93. The preparation of claim 63, comprising an emblica extract, a chebula extract, or a fucus extract fortified with the treatment agent.

94. A method of treating or preventing a disease or condition associated with mitochondrial dysfunction in a subject, the method comprising administering to the subject a preparation comprising an effective amount of an emblica extract, a chebula extract, a treatment agent comprising an isolated compound constituent of emblica extract, chebula extract, or a metabolite thereof, a compound having a similarity score of at least 95% with the isolated compound constituent of emblica extract, chebula extract, or metabolite thereof, a pharmaceutically acceptable form thereof, or a combination thereof.

95. The method of claim 94, wherein the disease or condition associated with mitochondrial dysfunction is a cardiovascular disease, diabetes, cancer, neurological disorder, skin disease or condition, hair or scalp disease or condition, or symptom thereof.

96. The method of claim 94, wherein the disease or condition associated with mitochondrial dysfunction is aging, an aging-associated chronic disease or condition, reduced energy levels and vitality, or symptom thereof.

97. The method of claim 94, wherein the effective amount is a therapeutically effective amount.

98. The method of claim 94, wherein the effective amount is sufficient to induce mitochondrial biogenesis.

99. The method of claim 94, wherein the treatment or prevention involves inducing mitochondrial biogenesis and/or improving mitochondrial function.

100. The method of claim 94, wherein administration increases expression of at least one protein selected from PGC-1a, TFAM, NRF-1, and COX II.

101. The method of claim 94, wherein administration activates a gene associated with mitochondrial activity.

102. The method of claim 94, wherein administration alters, e.g., decreases expression of at least one gene selected from the group consisting of: NF-κB, COX-2, INF-β1, CCL5, MMP1, MMP2, MMP9, MMP13, IGF1R, VEGF, and MRPS5, alters, e.g., increases expression of at least one gene selected from the group consisting of: TIMP1, KLOTHO, COL1A1, MTCO2, TFAM, and VDAC, or activates a gene selected from: FGF2, FGFR1, COX7A1, PDK4, FAM173A, MRPL12, and WNT11.

103. The method of claim 94, wherein the preparation is administered topically.

104. The method of claim 103, wherein the preparation is formulated as a topical solution, oil, cream, emulsion, foam, or gel.

105. The method of claim 103, wherein the preparation is formulated as a shampoo, conditioner, spray, cream, foam, gel, balm, body wash, soap, lotion, or make-up.

106. The method of claim 94, wherein the preparation is administered parenterally.

107. The method of claim 94, wherein the preparation is administered enterally.

108. The method of claim 94, wherein the preparation is administered locally.

109. The method of claim 94, wherein the preparation is administered systemically.

110. The method of claim 94, wherein the treatment agent is formulated in a nanoparticle-based delivery carrier or conjugated to a nanoparticle-based delivery carrier.

111. The preparation of claim 110, wherein the nanoparticle-based delivery carrier has an average size of about 10 to 5000 nm.

112. The preparation of claim 110, wherein the nanoparticle-based delivery carrier comprises and/or is functionalized with a carbon-based nanomaterial, liposomal delivery vehicle, polymeric nanocarrier, micelle, dendrimer, lipophilic cation, solid-lipid nanoparticle (SLN), peptide-based nanomaterial, nanostructured lipid carrier (NLC), niosome, nanoemulsion, metal nanoparticle, nanosphere, polymerosome, cubosome, or a combination thereof.

113. The method of claim 110, wherein the nanoparticle-based delivery carrier is functionalized with a mitochondrial targeting agent.

114. The preparation of claim 113, wherein the mitochondrial targeting agent is selected from a mitochondrial targeting antibody, a polymeric functional moiety (e.g., polyethylene glycol (PEG)), a lipophilic cation (e.g., triphenylphosphine (TPP)), a mitochondrial targeting peptide (MPP), a derivative thereof, or a combination thereof.

115. The method of claim 94, wherein the preparation is administered in combination with a skin penetration enhancer, e.g., a chemical skin penetration enhancer or a physical skin penetration enhancer.

116. The method of claim 115, wherein the chemical skin penetration enhancer comprises niosomes, proniosomes, liposomes, phospholipids, glycerin, alcohols, e.g., ethanol or glycol, sulfoxides, e.g., dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes or terpenoids, or a combination thereof.

117. The method of claim 115, wherein the physical skin penetration enhancer comprises a roller, scraper, scrubber, exfoliator, microdermabrasion needles, iontophoresis device, electroporation device, ultrasound device, e.g., sonophoresis device, thermal ablation, magnetophoresis, photomechanical waves, electron beam irradiation, low light therapy device, e.g., a light emitting diode (LED) source, or a combination thereof.

118. The method of claim 94, further comprising administering to the subject a second or subsequent amount of the preparation.

119. The method of claim 118, wherein the second or subsequent amount is administered as a second dose of the same formulation.

120. The method of claim 118, wherein the second or subsequent amount is administered in another formulation.

121. The method of claim 94, comprising administering the preparation to the subject in combination with caffeine, a B vitamin, e.g., B1, B2, B3, B5, B6, B8, B9 and/or B12 vitamin, vitamin C, iron, magnesium, zinc, a UV-blocking agent, moisturizer, sunscreen, wrinkle cream, retinoid, alpha-hydroxy acid, beta-hydroxy acid, squalene, antioxidant, e.g., CoQ10, vitamin E, carotenoid, e.g., beta-carotene, mineral, e.g., selenium or manganese, glutathione, lipoic acid, flavonoid, betaflavonoid, phenol, polyphenol, phytoestrogen, mitoquinol mesylate, ubiquinone, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, squalene, soy, dermal filler or injection, e.g. hyaluronic acid or calcium hydroxylapatite, botulinum toxin, laser resurfacing procedure, ultrasound therapy, chemical peel, e.g., glycolic acid peel, trichloroacetic acid or salicylic acid, dermabrasion procedure, or a combination thereof.

122. The preparation of claim 94, comprising administering the preparation to the subject in combination with a second agent approved to treat or commonly used to treat mitochondrial dysfunction, an inflammatory response, or inflammation.

123. A method of treating or preventing a skin disease or condition in a subject, the method comprising administering to the subject a preparation comprising an effective amount of an emblica extract, a chebula extract, a treatment agent comprising an isolated compound constituent of emblica extract, chebula extract, or a metabolite thereof, a compound having a similarity score of at least 95% with the isolated compound constituent of emblica extract, chebula extract, or metabolite thereof, a pharmaceutically acceptable form thereof, or a combination thereof.

124. The method of claim 123, wherein the skin disease or condition is a characteristic of skin aging, skin wrinkles, change in skin pigmentation, senile lentigines, or a disease or condition of sebaceous glands.

125. The method of claim 123, wherein administration promotes firmness, hydration, elasticity, radiance, tone evenness, visual smoothness, or tactile smoothness of skin of the subject.

126. The method of claim 123, wherein the effective amount is a therapeutically effective amount.

127. The method of claim 123, wherein the effective amount is sufficient to induce mitochondrial biogenesis.

128. The method of claim 123, wherein the treatment or prevention involves inducing mitochondrial biogenesis and/or improving mitochondrial function.

129. The method of claim 123, wherein administration increases expression of at least one protein selected from PGC-1a, TFAM, NRF-1, and COX II.

130. The method of claim 123, wherein administration activates a gene associated with mitochondrial activity.

131. The method of claim 123, wherein administration alters, e.g., decreases expression of at least one gene selected from the group consisting of: NF-κB, COX-2, INF-β1, CCL5, MMP1, MMP2, MMP9, MMP13, IGF1R, VEGF, and MRPS5, alters, e.g., increases expression of at least one gene selected from the group consisting of: TIMP1, KLOTHO, COL1A1, MTCO2, TFAM, and VDAC, or activates a gene selected from: FGF2, FGFR1, COX7A1, PDK4, FAM173A, MRPL12, and WNT11.

132. The method of claim 123, wherein the preparation is administered topically.

133. The method of claim 132, wherein the preparation is formulated as a topical solution, oil, cream, emulsion, foam, or gel.

134. The method of claim 132, wherein the preparation is formulated as a shampoo, conditioner, spray, cream, foam, gel, balm, body wash, soap, lotion, or make-up.

135. The method of claim 123, wherein the preparation is administered parenterally.

136. The method of claim 123, wherein the preparation is administered enterally.

137. The method of claim 123, wherein the preparation is administered locally.

138. The method of claim 123, wherein the preparation is administered systemically.

139. The method of claim 123, wherein the treatment agent is formulated in a nanoparticle-based delivery carrier or conjugated to a nanoparticle-based delivery carrier.

140. The preparation of claim 139, wherein the nanoparticle-based delivery carrier has an average size of about 10 to 5000 nm.

141. The preparation of claim 139, wherein the nanoparticle-based delivery carrier comprises and/or is functionalized with a carbon-based nanomaterial, liposomal delivery vehicle, polymeric nanocarrier, micelle, dendrimer, lipophilic cation, solid-lipid nanoparticle (SLN), peptide-based nanomaterial, nanostructured lipid carrier (NLC), niosome, nanoemulsion, metal nanoparticle, nanosphere, polymerosome, cubosome, or a combination thereof.

142. The method of claim 139, wherein the nanoparticle-based delivery carrier is functionalized with a mitochondrial targeting agent.

143. The preparation of claim 142, wherein the mitochondrial targeting agent is selected from a mitochondrial targeting antibody, a polymeric functional moiety (e.g., polyethylene glycol (PEG)), a lipophilic cation (e.g., triphenylphosphine (TPP)), a mitochondrial targeting peptide (MPP), a derivative thereof, or a combination thereof.

144. The method of claim 123, wherein the preparation is administered in combination with a skin penetration enhancer, e.g., a chemical skin penetration enhancer or a physical skin penetration enhancer.

145. The method of claim 144, wherein the chemical skin penetration enhancer comprises niosomes, proniosomes, liposomes, phospholipids, glycerin, alcohols, e.g., ethanol or glycol, sulfoxides, e.g., dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes or terpenoids, or a combination thereof.

146. The method of claim 144, wherein the physical skin penetration enhancer comprises a roller, scraper, scrubber, exfoliator, microdermabrasion needles, iontophoresis device, electroporation device, ultrasound device, e.g., sonophoresis device, thermal ablation, magnetophoresis, photomechanical waves, electron beam irradiation, low light therapy device, e.g., a light emitting diode (LED) source, or a combination thereof.

147. The method of claim 123, further comprising administering to the subject a second or subsequent amount of the preparation.

148. The method of claim 147, wherein the second or subsequent amount is administered as a second dose of the same formulation.

149. The method of claim 147, wherein the second or subsequent amount is administered in another formulation.

150. The method of claim 123, comprising administering the preparation to the subject in combination with caffeine, a B vitamin, e.g., B1, B2, B3, B5, B6, B8, B9 and/or B12 vitamin, vitamin C, iron, magnesium, zinc, a UV-blocking agent, moisturizer, sunscreen, wrinkle cream, retinoid, alpha-hydroxy acid, beta-hydroxy acid, squalene, antioxidant, e.g., CoQ10, vitamin E, carotenoid, e.g., beta-carotene, mineral, e.g., selenium or manganese, glutathione, lipoic acid, flavonoid, betaflavonoid, phenol, polyphenol, phytoestrogen, mitoquinol mesylate, ubiquinone, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, squalene, soy, dermal filler or injection, e.g. hyaluronic acid or calcium hydroxylapatite, botulinum toxin, laser resurfacing procedure, ultrasound therapy, chemical peel, e.g., glycolic acid peel, trichloroacetic acid or salicylic acid, dermabrasion procedure, or a combination thereof.

151. The preparation of claim 123, comprising administering the preparation to the subject in combination with a second agent approved to treat or commonly used to treat the skin disease or condition, mitochondrial dysfunction, an inflammatory response, or inflammation.

152. A method of treating or preventing a scalp or hair disease or condition in a subject, the method comprising administering to the subject a preparation comprising an effective amount of an emblica extract, a chebula extract, a treatment agent comprising an isolated compound constituent of emblica extract, chebula extract, or a metabolite thereof, a compound having a similarity score of at least 95% with the isolated compound constituent of emblica extract, chebula extract, or metabolite thereof, a pharmaceutically acceptable form thereof, or a combination thereof.

153. The method of claim 152, wherein the scalp or hair disease or condition is hair loss, hair thinning, or change in hair pigmentation.

154. The method of claim 153, wherein the hair loss comprises total alopecia, partial alopecia, or male or female pattern baldness.

155. The method of claim 153, wherein the hair loss is a genetic condition or associated with an autoimmune disease, an environmental factor, or a course of treatment.

156. The method of claim 152, wherein the effective amount is a therapeutically effective amount.

157. The method of claim 152, wherein the effective amount is sufficient to induce mitochondrial biogenesis.

158. The method of claim 152, wherein the treatment or prevention involves inducing mitochondrial biogenesis and/or improving mitochondrial function.

159. The method of claim 152, wherein administration increases expression of at least one protein selected from PGC-1a, TFAM, NRF-1, and COX II.

160. The method of claim 152, wherein administration activates a gene associated with mitochondrial activity.

161. The method of claim 152, wherein administration alters, e.g., decreases expression of at least one gene selected from the group consisting of: NF-κB, COX-2, INF-β1, CCL5, MMP1, MMP2, MMP9, MMP13, IGF1R, VEGF, and MRPS5, alters, e.g., increases expression of at least one gene selected from the group consisting of: TIMP1, KLOTHO, COL1A1, MTCO2, TFAM, and VDAC, or activates a gene selected from: FGF2, FGFR1, COX7A1, PDK4, FAM173A, MRPL12, and WNT11.

162. The method of claim 152, wherein the preparation is administered topically.

163. The method of claim 162, wherein the preparation is formulated as a topical solution, oil, cream, emulsion, foam, or gel.

164. The method of claim 162, wherein the preparation is formulated as a shampoo, conditioner, spray, cream, foam, gel, balm, body wash, soap, lotion, or make-up.

165. The method of claim 152, wherein the preparation is administered parenterally.

166. The method of claim 152, wherein the preparation is administered enterally.

167. The method of claim 152, wherein the preparation is administered locally.

168. The method of claim 152, wherein the preparation is administered systemically.

169. The method of claim 152, wherein the treatment agent is formulated in a nanoparticle-based delivery carrier or conjugated to a nanoparticle-based delivery carrier.

170. The preparation of claim 169, wherein the nanoparticle-based delivery carrier has an average size of about 10 to 5000 nm.

171. The preparation of claim 169, wherein the nanoparticle-based delivery carrier comprises and/or is functionalized with a carbon-based nanomaterial, liposomal delivery vehicle, polymeric nanocarrier, micelle, dendrimer, lipophilic cation, solid-lipid nanoparticle (SLN), peptide-based nanomaterial, nanostructured lipid carrier (NLC), niosome, nanoemulsion, metal nanoparticle, nanosphere, polymerosome, cubosome, or a combination thereof.

172. The method of claim 169, wherein the nanoparticle-based delivery carrier is functionalized with a mitochondrial targeting agent.

173. The preparation of claim 172, wherein the mitochondrial targeting agent is selected from a mitochondrial targeting antibody, a polymeric functional moiety (e.g., polyethylene glycol (PEG)), a lipophilic cation (e.g., triphenylphosphine (TPP)), a mitochondrial targeting peptide (MPP), a derivative thereof, or a combination thereof.

174. The method of claim 152, wherein the preparation is administered in combination with a skin penetration enhancer, e.g., a chemical skin penetration enhancer or a physical skin penetration enhancer.

175. The method of claim 174, wherein the chemical skin penetration enhancer comprises niosomes, proniosomes, liposomes, phospholipids, glycerin, alcohols, e.g., ethanol or glycol, sulfoxides, e.g., dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes or terpenoids, or a combination thereof.

176. The method of claim 174, wherein the physical skin penetration enhancer comprises a roller, scraper, scrubber, exfoliator, microdermabrasion needles, iontophoresis device, electroporation device, ultrasound device, e.g., sonophoresis device, thermal ablation, magnetophoresis, photomechanical waves, electron beam irradiation, low light therapy device, e.g., a light emitting diode (LED) source, or a combination thereof.

177. The method of claim 152, further comprising administering to the subject a second or subsequent amount of the preparation.

178. The method of claim 177, wherein the second or subsequent amount is administered as a second dose of the same formulation.

179. The method of claim 177, wherein the second or subsequent amount is administered in another formulation.

180. The method of claim 152, comprising administering the preparation to the subject in combination with caffeine, a B vitamin, e.g., B1, B2, B3, B5, B6, B8, B9 and/or B12 vitamin, vitamin C, iron, magnesium, zinc, a UV-blocking agent, moisturizer, sunscreen, wrinkle cream, retinoid, alpha-hydroxy acid, beta-hydroxy acid, squalene, antioxidant, e.g., CoQ10, vitamin E, carotenoid, e.g., beta-carotene, mineral, e.g., selenium or manganese, glutathione, lipoic acid, flavonoid, betaflavonoid, phenol, polyphenol, phytoestrogen, mitoquinol mesylate, ubiquinone, tretinoin, glycosaminoglycan (GAG), lactic acid, malic acid, citric acid, tartaric acid, hydroquinone, kojic acid, L-ascorbic acid, licorice extract, N-acetylglucosamine, niacinamide, squalene, soy, dermal filler or injection, e.g. hyaluronic acid or calcium hydroxylapatite, botulinum toxin, laser resurfacing procedure, ultrasound therapy, chemical peel, e.g., glycolic acid peel, trichloroacetic acid or salicylic acid, dermabrasion procedure, or a combination thereof.

181. The preparation of claim 152, comprising administering the preparation to the subject in combination with a second agent approved to treat or commonly used to treat the scalp or hair disease or condition, mitochondrial dysfunction, an inflammatory response, or inflammation.
